(19) United States
(12) Patent Application Publication
(10) Pub. No.: US 2026/0273028 A1
(43) Pub. Date: Sep. 17, 2026

(54) PEROXIDASE COMPOSITIONS CONTAINING IMPROVED ENHANCING AGENTS

(71) Applicant: FLEN HEALTH PHARMA NV, Kontich (BE)

(72) Inventors: Gilles Brackman, Scheldewindeke (BE); Youtha Van Den Eynde, Willebroek (BE); Eveline Torfs, Edegem (BE); Philippe Sollie, Luxembourg (LU)

(21) Appl. No.: 19/128,891

(22) PCT Filed: Nov. 16, 2023
(86) PCT No.: PCT/EP2023/082014

§ 371 Date: May 9, 2025

(30) Foreign Application Priority Data
Nov. 16, 2022 (EP) 22207660.6

(51) Int. Cl. A61K 38/44; A61K 47/02; A61K 47/18; A61P 29/00; A61P 31/04; A61P 37/06

(52) U.S. Cl. CPC A61K 38/443; A61K 47/02; A61K 47/183; A61P 29/00; A61P 31/04; A61P 37/06; C12Y 101/03004; C12Y 111/01007

(57) ABSTRACT

The present invention provides peroxidase-based pharmaceutical, prophylactic, and cosmetic compositions. The compositions are preferably formulated to accommodate a topical use. More particularly, the invention relates to peroxidase-based compositions further comprising a compound that enhances and/or stabilizes the activity of antimicrobial peroxidases. The invention further provides methods and uses involving the compositions of the invention.

### CROSS REFERENCE TO RELATED APPLICATIONS

This application is a national phase application of International Application No. PCT/EP2023/082014, filed 16 Nov. 2023, which claims priority to European App. No. 22207660.6, filed 16 Nov. 2022, the contents of each are incorporated by reference herein in their entirety for all purposes.

### FIELD OF THE INVENTION

The present invention is situated in the technical fields of medicine and biomedical science. The present invention broadly relates to both pharmaceutical and cosmetic compositions, and related methods and uses thereof. More particularly, the invention relates to peroxidase-based compositions further comprising a compound that enhances and/or stabilises the activity of antimicrobial peroxidases.

### BACKGROUND OF THE INVENTION

Peroxidases are ubiquitous enzymes that are part of the larger group of oxidoreductases and constitute the second largest class of enzymes applied in biotechnological processes. Peroxidases are used to catalyse various oxidative reactions using hydrogen peroxide and other substrates as electron donors and this reduction of peroxides at the expense of electron donating substrates renders peroxidases particularly useful for a plethora of biotechnological applications. Different peroxidases can be isolated from various sources such as plants, animals and microbes. Peroxidase enzymes have versatile applications in bioenergy, bioremediation, dye decolorization, humic acid degradation, paper and pulp, and textile industries (Twala et al., AIMS Microbiol, 2020).

The antimicrobial activity of peroxidase-based systems depends on the type of electron donor being used. EP514417 reports on anti-microbial compositions comprising glucose oxidase, iodide and thiocyanate ions. EP514417 further demonstrates an enhancing effect of antioxidants on the peroxidase activity.

U.S. Pat. No. 4,476,108 describes methods for producing bactericidal free radicals in the mouth over a controlled time period by applying a combination of a peroxidase, a peroxide and a source of donor molecules. The preparation is stated to be preferably used in a carrier liquid or paste. The carrier can be water, toothpaste, mouthwash, chewing gum, prophylaxis paste, denture cleaner, and oral cleansing gel. More particularly, U.S. Pat. No. 4,476,108 discloses antimicrobial compositions for oral application with a short-term activity (less than two minutes) using peroxide, a peroxidase, a donor molecule such as phenylethylamine, tyrosine, tryptophan, benzoic acid, salicylic acid, hydroquinone, dehydrophenylalanine, vanillin and para-aminobenzoic acid.

Antimicrobial activity against fungi and bacteria has been attributed to aromatic flavouring compounds such as syringaldehyde and vanillin (Fitzgerald et al., J Agric Food Chem, 2005; and Fitzgerald et al., J Appl Microbial, 2004). The antimicrobial activity is however only observed at high concentrations and after prolonged periods of incubation.

US 20020119136 describes a class of dialkoxyphenol compounds (e.g. acetosyringone and alkylsyringates) with an enhancing effect on peroxidases.

WO 2006/133523 describes improved anti-microbial peroxidase-based composition comprising benzene compounds substituted with a —OH or a (CH₂)ₙOH group (n=1, 2, 3 or 4) and substituted with one alkoxy group (—OR) with a chain length of 1, 2, 3 or 4 carbon atoms (including guaiacol). These enhancers are currently incorporated in distinct commercialised compositions such as Flaminal® Forte and Flaminal® Hydro, both produced by Flen Health for the treatment of acute and chronic wound types (e.g., traumatic wounds, burns, surgical wounds, venous and arterial ulcers, diabetic ulcers, pressure ulcers, . . . ). Nevertheless, given the highly specific nature of these enhancing agents, alternative and/or improved enhancing agents would be valuable to further improve peroxidase-based compositions.

There is therefore an unmet need for further improved potent and/or stable peroxidase-based antimicrobial compositions.

### SUMMARY OF THE INVENTION

As evidenced in detail by the examples enclosed herewith, the inventors have found a group of enhancing agents such as certain amino acids, amino acid mimics, and amino acid derivatives that are particularly useful for improving the peroxide generating system in antimicrobial compositions. In addition to said enhancing agents, the compositions comprise a peroxide generating system, a peroxidase, and a halide or pseudohalide. The inventors provide experimental evidence for the potency of said enhancing agents by means of both enzymatic assays and antimicrobial activity assays (Minimal Inhibitory Concentration (MIC) assays, Minimal Bactericidal Concentration (MBC) assays, absolute bacterial counting experiments, and challenge testing). This finding is directly applicable to any kind of composition such as but not limited to therapeutic compositions, prophylactic compositions, and cosmetic compositions, and more particularly topical compositions. Compositions comprising the above constituents are therefore capable of increasing the speed of healing by modulation of the microbial load, if present, and by modulation of the immune response via multiple mechanisms (both indirect and direct) as outlined below.

Firstly, the compositions described herein modulate the microbial load and thus indirectly also the inflammatory state, even when compared to known compositions that rely on other enhancing agents such as but not limited to guaiacol. Indeed, by reducing the microbial load (i.e. reducing the number of microbes present in an infected wound) the composition effectively tempers the inflammatory state in an indirect manner.

In addition, the compositions described herein also modulate inflammation in a direct manner, i.e. both by decreasing the secretion of pro-inflammatory molecules by immune cells and by exerting a neutralizing effect on already secreted pro-inflammatory molecules. Hence, even in absence of an infection the compositions described herein will exert a direct effect on inflammation by either reducing the secretion of pro-inflammatory markers (i.e. molecules) by immune cells, neutralizing already secreted pro-inflammatory markers, or a combination thereof.

Taken together the above findings made by the inventors, the compositions described herein are potent means to increase the speed of healing of wounds by each of the above properties, and particularly the combined effect of the above properties, ultimately resulting in reduction of discomfort, mortality, and morbidity of subjects. A further consequence of the antimicrobial effect is that when the composition is applied to a subject as a component of an ointment, gel, dressing, or the like needs to be less frequently replaced.

Moreover, the inventors unexpectedly found that certain halides such as potassium iodide further improved the composition to a greater extent when compared to other halides, including those commonly used in the art. Finally, lactoperoxidase was found to be a particularly potent peroxidase when used in the compositions described herein.

Accordingly, a first aspect of the invention is therefore directed to compositions comprising a peroxide generating system, a peroxidase, a halide or pseudohalide, and an enhancing agent, wherein the enhancing agent is an organic compound characterised by (a) an amino and carboxylate functional group and further comprising one or more side chains which comprise a functional group selected from the group consisting of: polar uncharged methanol, ethan-1-ol, acetamide, propenamide, or a secondary butyl group; or (b) a pyrrolidine and carboxylate functional group.

In particular embodiments, the connection between the amino and/or carboxylate functional group and the different side chains or the pyrrolidine (in a and b) consists of (CH₂)ₙ where n=1, 2 or 3.

In particular embodiments, the enhancing agent is a hydrophilic amino acid, amino acid mimic, or amino acid derivative, characterized by a free amino functional group (—NH₃⁺), free carboxylic acid functional group (—CO₂H), and a side chain comprising a
(i) —OH group;
(ii) —(CH2)ₙOH group wherein n is 1, 2, or 3;
(iii) —(CH2)ₙ CONH₂ group wherein n is 1, 2, or 3;
(iv) -a cyclic C₄H₉N group; or
(v) a branched alkylgroup.

In certain embodiments, the enhancing agent is a hydrophilic amino acid, amino acid mimic, or amino acid derivative having an octanol/water partition coefficient of from about −1 to about −5.

In certain embodiments, the enhancing agent is a hydrophilic naturally or non-naturally occurring amino acid.

In certain embodiments, the enhancing agent is a naturally occurring amino acid having an octanol/water partition coefficient of from about −1.72 to about −3.82.

In certain embodiments, the halide or pseudohalide is a water-soluble iodide salt. Preferably, the halide is potassium iodide which was found to further improve the antimicrobial activity of the composition.

In certain embodiments, the enhancing agent is a naturally occurring amino acid selected from the group consisting of: serine, threonine, asparagine, glutamine, isoleucine, proline, or any combination thereof.

In certain embodiments, the enhancing agent is present in said composition at a concentration of from 0.0005 mg/ml to 200 mg/ml, preferably of from 0.005 mg/ml to 10 mg/ml, most preferably of from 0.05 to 1 mg/ml.

In certain embodiments, the peroxide generating system comprises an oxidase, preferably the peroxide generating system is glucose oxidase.

In certain embodiments, the peroxidase is lactoperoxidase which was found to function particularly well with the enhancing agents described herein. In certain embodiments, the lactoperoxidase is human lactoperoxidase or bovine peroxidase. Preferably, the lactoperoxidase comprises an amino acid sequence having at least 80%, preferably at least 85%, preferably at least 90%, more preferably at least 95%, most preferably 100% sequence identity to SEQ ID NO: 1.

In certain embodiments, the composition further comprises one or more solvents, diluents, buffers, solubilizers, colloids, fillers, dispersion mediums, amino acids, proteins, disintegrants, binders, lubricants, wetting agents, stabilizers, emulsifiers, sweeteners, colouring agents, flavouring, coatings, antifungal agents, preservatives, antioxidants, adjuvants, viscosity modifiers, permeation enhancers, chelators or any combination thereof. In a further embodiment, the composition further comprises guaiacol.

In certain embodiments, the enhancing agent increases the activity of the peroxide generating system and/or the peroxidase by at least 10%, preferably at least 20%, more preferably at least 30%, even more preferably at least 40%, most preferably at least 50% when compared to a reference (control) composition that does not comprise an enhancing agent.

In certain embodiments, the composition is characterised by a Minimum Inhibitory Concentration (MIC) and/or a Minimal Bactericidal Concentration (MBC) and/or absolute counts and/or enzymatic activity for an inoculum of S. aureus, P. aeruginosa, C. albicans and A. niger or any combination thereof that is significantly reduced when compared to a reference (control) composition that does not comprise an enhancing agent.

In certain embodiments, the MIC and/or MBC is determined by incubation of said composition for 24 hours at 37° C. (S. aureus, P. aeruginosa and C. albicans) or for 48 hours at 25° C. (A. niger) in a microbial inoculum characterised by an OD₆₀₀ of 0.1.

In certain embodiments, the composition is a topical composition. In preferred embodiments, said topical composition is for cutaneous application.

In certain embodiments, the composition is a cream, lotion, foundation, ointment, suspension (oil-in-water and water-in-oil), patch, dressing, gel, or emulsion. In certain embodiments, the composition is a deodorant, preferably a roll-on or stick deodorant. In certain embodiments, the composition is an anti-acne preparation, preferably in the form of a lotion or a cream. In certain embodiments, the composition is comprised in or applied to an impregnated materials such as wound dressings. In further embodiments, the wound dressing is a dressing configured for wound irrigation and/or treatment of wounds. In certain embodiments, the composition is an ear drop formulation, nose drop formulation, inhalation formulation, vaginal formulation, or rectal solution.

In certain embodiments, the composition comprises:
from about 150 to about 4000 U/kg, preferably of from about 200 to about 3000 U/kg, more preferably from about 300 to about 2500 U/kg of the peroxide generating system;
from about 10 to 100000 U/kg, preferably from about 10 to 4000 U/kg, more preferably from about 10 to 100 U/kg of the peroxidase such as lactoperoxidase;
from about 0.01 to about 500 mg/kg, preferably from about 0.1 to about 200 mg/kg, more preferably from about 1 mg to about 100 mg/kg, yet more preferably from about 2 mg to about 75 mg/kg, most preferably from 5 to 50 mg/kg of the halide or pseudohalide;
and from 0.0005 mg/ml to 200 mg/ml, preferably of from 0.005 mg/ml to 10 mg/ml, more preferably of from 0.05 to 1 mg/ml of the enhancing agent.

In certain embodiments, the composition is a pharmaceutical or cosmetic composition.

In certain embodiments, the composition is a sustained or controlled release composition.

In a further aspect, the composition described herein is envisaged for use as a medicament. In certain embodiments, the composition described herein is envisaged for use in treatment or prevention of a skin disorder. In certain embodiments, the composition described herein is envisaged for use in wound healing. In certain embodiments, the composition described herein is envisaged for use as an antimicrobial composition or for the treatment or prevention of microbial infections. In yet further aspects, the composition described herein is envisaged for use as an anti-inflammatory medicament, i.e., a for use in reducing inflammation in a subject.

In a further aspect, the use of the composition described herein is envisaged for the manufacture of a medicament. In certain embodiments, the use of the composition described herein is envisaged for the manufacture of a medicament for the treatment or prevention of skin disorder. In certain embodiments, the use of the composition described herein is envisaged for the manufacture of a medicament for the treatment of wounds. In certain embodiments, the use of the composition described herein is envisaged for the manufacture of a medicament for the treatment or prevention of microbial infections.

In a further aspect, the invention envisages a method of treating or preventing a skin disorder in a subject, comprising administering to said subject the composition described herein. In certain embodiments, the invention concerns a method of treating a wound, comprising administering to said subject the composition described herein. In certain embodiments, the invention concerns a method of treating or preventing a microbial infection, comprising administering to said subject the composition described herein. In certain embodiments, the invention concerns of an indirect method of treating or preventing inflammation that is related to the presence of microbial infection, comprising administering to said subject the composition described herein.

In yet a further aspect, the invention is directed to the use of an enhancing agent for improving the Minimum Inhibitory Concentration (MIC) and/or the Minimum Bactericidal concentration (MBC) and/or absolute counts and/or enzymatic activity of a pharmaceutical composition comprising a peroxide generating system, a peroxidase, and a halide or pseudohalide, wherein the enhancing agent is a hydrophilic amino acid, amino acid mimic, or amino acid derivative, characterized by a free amino functional group (—NH₃⁺), free carboxylic acid functional group (—CO₂H), and a side chain comprising a
(i) —OH group;
(ii) —(CH2)ₙOH group wherein n is 1, 2, or 3;
(iii) —(CH2)ₙCONH₂ group wherein n is 1, 2, or 3;
(iv) -a cyclic C₄H₉N group; or
(v) a branched alkylgroup.

In certain embodiments the enhancing agent is a naturally or non-naturally occurring amino acid, preferably the enhancing agent is a naturally occurring amino acid selected from the group consisting of: serine, threonine, asparagine, glutamine, isoleucine, proline, or any combination thereof.

In certain embodiments the use is an in vitro use. In certain embodiments, the use is an in vitro use for the preservation of skin grafts, cell lines, biomarkers, and/or biological sample material.

In yet a further aspect, the invention is directed to the use of an enhancing agent to preserve sterility of a pharmaceutical formulation, wherein the enhancing agent is as described herein above. In particular embodiments, the enhancing agent is a hydrophilic amino acid, amino acid mimic, or amino acid derivative, characterized by a free amino functional group (—NH₃⁺), free carboxylic acid functional group (—CO₂H), and a side chain comprising a
(i) —OH group;
(ii) —(CH2)ₙOH group wherein n is 1, 2, or 3;
(iii) —(CH2)ₙCONH₂ group wherein n is 1, 2, or 3;
(iv) -a cyclic C₄H₉N group; or
(v) a branched alkylgroup.

The above and further aspects and preferred embodiments of the invention are described in the following sections and in the appended claims. The subject matter of the appended claims is hereby specifically incorporated in this specification.

### BRIEF DESCRIPTION OF THE FIGURES

FIG. 1. Overview of GOX activity in the presence of proline (100-0.1 mg/ml) and cysteine (1-0.1 mg/ml). Each result represents the mean enzyme activity (expressed as U/g)+SD of at least 2 biological repeats. Statistical significance compared to the reference (i.e. GOX activity w/o enhancing molecule candidate) was calculated using students' t-tests. Significance levels are defined as p<0,05=*; P<0,01=** and p<0,001=***.

FIG. 2. Overview of LPO activity in the presence of different molecules. Each result represents the mean enzyme activity (expressed as U/g)+SD of at least 2 biological repeats. Statistical significance compared to the reference (LPO activity w/o enhancing molecule candidate) was calculated using students' t-tests. Significance levels are defined as p<0,05=*; P<0,01=** and p<0,001=***.

FIG. 3. Overview of absolute counts of S. aureus in the presence of serine and isoleucine as potentiator candidate. Each result represents the average derived from 3 to 7 biological repeats. X-axis: concentration enzyme (%); Y-axis: microbial concentration (log CFU/ml).

FIG. 4. Overview of absolute counts of S. aureus in the presence of serine, glutamine and isoleucine as potentiator candidate. Each result represents the average derived from 3 to 7 biological repeats. X-axis: concentration enzyme (%); Y-axis: microbial concentration (log CFU/ml).

FIG. 5. Overview of absolute counts of P. aeruginosa in the presence of serine and proline as potentiator candidate. Each result represents the average derived from 3 to 7 biological repeats. X-axis: concentration enzyme (%); Y-axis: microbial concentration (log CFU/ml).

FIG. 6. Overview of absolute counts of P. aeruginosa in the presence of serine and proline as potentiator candidate. Each result represents the average derived from 3 to 7 biological repeats. X-axis: concentration enzyme (%); Y-axis: microbial concentration (log CFU/ml).

FIG. 7. Overview of absolute counts of P. aeruginosa in the presence of threonine as potentiator candidate. Each result represents the average derived from 2 to 3 biological repeats. X-axis: concentration enzyme (%); Y-axis: microbial concentration (log CFU/ml).

FIG. 8. Overview of absolute counts of P. aeruginosa in the presence of isoleucine as potentiator candidate.

Each result represents the average derived from 2 to 3 biological repeats. X-axis: concentration enzyme (%); Y-axis: microbial concentration (log CFU/ml).

FIG. 9. Overview of absolute counts of P. aeruginosa in the presence of glutamine as potentiator candidate.

Each result represents the average derived from 2 to 3 biological repeats. X-axis: concentration enzyme (%); Y-axis: microbial concentration (log CFU/ml).

FIG. 10. Overview of absolute counts of P. aeruginosa in the presence of asparagine as potentiator candidate. Each result represents the average derived from 2 to 3 biological repeats. X-axis: concentration enzyme (%); Y-axis: microbial concentration (log CFU/ml).

FIG. 11. Overview of absolute counts of C. albicans in the presence of serine and isoleucine as potentiator candidate. Each result represents the average derived from 3 to 7 biological repeats. X-axis: concentration enzyme (%); Y-axis: microbial concentration (log CFU/ml).

FIG. 12. Overview of absolute counts of A. niger in the presence of proline as potentiator candidate. Each result represents the average derived from 2 to 4 biological repeats. X-axis: concentration enzyme (%); Y-axis: microbial concentration (log CFU/ml).

FIG. 13. Overview of absolute counts of A. niger in the presence of serine as potentiator candidate. Each result represents the average derived from 2 to 4 biological repeats. X-axis: concentration enzyme (%); Y-axis: microbial concentration (log CFU/ml).

FIG. 14. Overview of absolute counts of A. niger in the presence of threonine as potentiator candidate. Each result represents the average derived from 2 to 4 biological repeats. X-axis: concentration enzyme (%); Y-axis: microbial concentration (log CFU/ml).

FIG. 15. Overview of absolute counts of A. niger in the presence of glutamine as potentiator candidate. Each result represents the average derived from 2 to 4 biological repeats. X-axis: concentration enzyme (%); Y-axis: microbial concentration (log CFU/ml).

FIG. 16. Overview of absolute counts of A. niger in the presence of asparagine as potentiator candidate. Each result represents the average derived from 2 to 4 biological repeats. X-axis: concentration enzyme (%); Y-axis: microbial concentration (log CFU/ml).

FIG. 17. Overview of absolute counts of A. niger in the presence of isoleucine as potentiator candidate. Each result represents the average derived from 2 to 4 biological repeats. X-axis: concentration enzyme (%); Y-axis: microbial concentration (log CFU/ml).

FIG. 18. Overview of absolute counts of S. aureus in the presence of proline as potentiator candidate. Each result represents the average derived from 1 to 3 biological repeats.

FIG. 19. Overview of absolute counts of P. aeruginosa in the presence of proline as potentiator candidate. Each result represents the average derived from 1 to 3 biological repeats.

FIG. 20. Overview of absolute counts of C. albicans in the presence of proline as potentiator candidate. Each result represents the average derived from 1 to 3 biological repeats.

FIG. 21. Overview of absolute counts of A. niger in the presence of proline as potentiator candidate. Each result represents the average derived from 1 to 3 biological repeats.

FIG. 22. Overview of absolute counts of S. aureus in the presence of serine as potentiator candidate. Each result represents the average derived from 1 to 3 biological repeats.

FIG. 23. Overview of absolute counts of P. aeruginosa in the presence of serine as potentiator candidate. Each result represents the average derived from 1 to 3 biological repeats.

FIG. 24. Overview of absolute counts of C. albicans in the presence of serine as potentiator candidate. Each result represents the average derived from 1 to 3 biological repeats.

FIG. 25. Overview of absolute counts of A. niger in the presence of serine as potentiator candidate. Each result represents the average derived from 1 to 3 biological repeats.

FIG. 26. Overview of absolute counts of S. aureus in the presence of threonine as potentiator candidate. Each result represents the average derived from 1 to 3 biological repeats.

FIG. 27. Overview of absolute counts of P. aeruginosa in the presence of threonine as potentiator candidate. Each result represents the average derived from 1 to 3 biological repeats.

FIG. 28. Overview of absolute counts of C. albicans in the presence of threonine as potentiator candidate. Each result represents the average derived from 1 to 3 biological repeats.

FIG. 29. Overview of absolute counts of A. niger in the presence of threonine as potentiator candidate. Each result represents the average derived from 1 to 3 biological repeats.

FIG. 30. Overview of absolute counts of S. aureus in the presence of glutamine as potentiator candidate. Each result represents the average derived from 1 to 3 biological repeats.

FIG. 31. Overview of absolute counts of P. aeruginosa in the presence of glutamine as potentiator candidate. Each result represents the average derived from 1 to 3 biological repeats.

FIG. 32. Overview of absolute counts of C. albicans in the presence of glutamine as potentiator candidate. Each result represents the average derived from 1 to 3 biological repeats.

FIG. 33. Overview of absolute counts of A. niger in the presence of glutamine as potentiator candidate. Each result represents the average derived from 1 to 3 biological repeats.

FIG. 34. Overview of absolute counts of S. aureus in the presence of asparagine as potentiator candidate. Each result represents the average derived from 1 to 3 biological repeats.

FIG. 35. Overview of absolute counts of P. aeruginosa in the presence of asparagine as potentiator candidate. Each result represents the average derived from 1 to 3 biological repeats.

FIG. 36. Overview of absolute counts of C. albicans in the presence of asparagine as potentiator candidate. Each result represents the average derived from 1 to 3 biological repeats.

FIG. 37. Overview of absolute counts of A. niger in the presence of asparagine as potentiator candidate. Each result represents the average derived from 1 to 3 biological repeats.

FIG. 38. Overview of absolute counts of S. aureus in the presence of isoleucine as potentiator candidate. Each result represents the average derived from 1 to 3 biological repeats.

FIG. 39. Overview of absolute counts of P. aeruginosa in the presence of isoleucine as potentiator candidate. Each result represents the average derived from 1 to 3 biological repeats.

FIG. 40. Overview of absolute counts of C. albicans in the presence of isoleucine as potentiator candidate. Each result represents the average derived from 1 to 3 biological repeats.

FIG. 41. Overview of absolute counts of A. niger in the presence of isoleucine as potentiator candidate. Each result represents the average derived from 1 to 3 biological repeats.

FIG. 42. Overview of absolute counts of S. aureus in the presence of ethanolamine or proline in 0,0005% as a potentiator candidate. The microbial load (expressed as log CFU/g alginogel) was plotted in function of time (expressed as minutes). Each result represents the average derived from 3 biological repeats.

FIG. 43. Overview of absolute counts of S. aureus in the presence of KI, KBr and KCl in a final concentration of 0,03 and 0,003%. The microbial load (expressed as log CFU/g alginogel) was plotted in function of time (expressed as minutes). Each result represents 1 biological repeat.

FIG. 44. Overview of IL-6 modulation. (A) Inhibition of IL-6 secretion by macrophages after 24 hours of no treatment, treatment with GOX+LPO (1:1) alone, or treatment with GOX+LPO (1:1) and isoleucine at 0.0025, 0.05 and 0.5%. Results were expressed as absolute IL-6 concentration (pg/ml), and relative IL-6 secretion compared to the no treatment group (%). (B) Neutralization of secreted IL-6 after 3 hours of no treatment, treatment with GOX+LPO (1:1) alone, or treatment with GOX+LPO (1:1) and isoleucine at 0.05%. Results were expressed as absolute IL-6 concentration (pg/ml), and relative IL-6 neutralization compared to the no treatment group (%).

FIG. 45. Overview of TNFa modulation. Neutralization of secreted TNFa after 3 hours of no treatment, treatment with GOX+LPO (1:1) alone, or treatment with GOX+LPO (1:1) and isoleucine at 0.05%. Results were expressed as absolute TNFa concentration (pg/ml), and relative TNFa neutralization compared to the no treatment group (%).

FIG. 46. Overview of MMP9 modulation. (A) Inhibition of MMP9 secretion by macrophages after 24 hours of no treatment, treatment with GOX+LPO (1:1) alone, or treatment with GOX+LPO (1:1) and isoleucine at 0.0025 and 0.05%. Results were expressed as absolute MMP9 concentration (pg/ml), and relative MMP9 secretion compared to the no treatment group (%) and represent the average of 2 biological repeats. (B) Neutralization of secreted MMP9 after 3 hours of no treatment, treatment with GOX+LPO (1:1) alone, or treatment with GOX+LPO (1:1) and isoleucine at 0.05%. Results were expressed as absolute MMP9 concentration (pg/ml), and relative MMP9 neutralization compared to the no treatment group (%).

FIG. 47. Overview of absolute counts of S. aureus after treatment with an alginogel with the GOX+EPO enzyme combination in the presence and absence of isoleucine at 0.0025% as a potentiator candidate. An alginogel without GOX+EPO enzyme combination and without isoleucine served as negative control. The microbial load (expressed as log(CFU/g alginogel)) was plotted in function of time (expressed as minutes). Each result is derived from a single biological repeat.

### DETAILED DESCRIPTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints. This applies to numerical ranges irrespective of whether they are introduced by the expression “from . . . to . . . ” or the expression “between . . . and . . . ” or another expression.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g. any ≥3, ≥4, ≥5, ≥6 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation or meaning is meant to apply throughout this specification, i.e. also in the context of other aspects or embodiments of the invention, unless otherwise defined. For example, embodiments directed to products are also applicable to corresponding features of methods and uses.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, alternative combinations of claimed embodiments are encompassed, as would be understood by those in the art.

Unless indicated otherwise, all methods, steps, techniques and manipulations that are not specifically described in detail can be performed and have been performed in a manner known per se, as will be clear to the skilled person. Reference is for example again made to standard handbooks as well as to the general background art referred to herein and to the further references cited therein.

In an earlier application of the Applicant (WO 2006/133523), enhancing agents have been described that enhance and/or stabilise the activity of antimicrobial peroxidases. Said disclosure teaches that a class of benzene compounds substituted with a —OH or a (CH₂)ₙOH group (n=1, 2, 3 or 4) and substituted with one alkoxy group (—OR) with a chain length of 1, 2, 3 or 4 carbon atoms (including guaiacol) have an enhancing effect on peroxidase-based antimicrobial compositions. The formulation of these composition comprises an antimicrobial enzyme system based on an oxidase enzyme (i.e., glucose oxidase or GOX) and a peroxidase enzyme (i.e., lactoperoxidase or “LPO”), which will in the presence of air, water and an iodide ion give rise to a reactive oxygen species (interchangeably indicated by the abbreviation “ROS”), namely hypoiodite, that will kill microbial pathogens in a non-specific manner. The enhancing molecule guaiacol is described to stabilize the ROS formed by LPO and prolong their antimicrobial reactivity. Similarly, other enhancing agents such as ethanolamine have been described in the art (e.g. in US 2015/196025 A1) for use in antimicrobial compositions relying on an oxidase enzyme and a peroxidase enzyme.

The present disclosure provides alternative and improved enhancing agents that that provide the beneficial effect of further improving the efficacy of peroxidase generating systems, and thus ultimately the peroxidases activity in the herein described compositions and uses. The enhancing agents described herein generally outperform the enhancing agents described in WO 2006/133523 (guaiacol) and US 2015/196025 A1 (ethanolamine).

Accordingly, in a first aspect the invention concerns composition comprising a peroxide generating system, a peroxidase, a halide or pseudohalide, and an enhancing agent, wherein the enhancing agent is an organic compound characterised by (a) an amino and carboxylate functional group and further comprising one or more side chains which comprise a functional group selected from the group consisting of: polar uncharged methanol, ethan-1-ol, acetamide, propenamide, or a secondary butyl group; or (b) a pyrrolidine and carboxylate functional group.

In particular embodiments, the connection between the amino and/or carboxylate functional group and the different side chains or the pyrrolidine (in a and b) consists of (CH2)ₙ where n=1, 2 or 3.

In particular embodiments, said compounds are hydrophilic, i.e. the partition coefficients of said compounds are between −1.72 and −3.82.

In particular embodiments, the enhancing agent is a hydrophilic amino acid, amino acid mimic, or amino acid derivative, characterized by a free amino functional group (—NH₃⁺), free carboxylic acid functional group (—CO₂H), and a side chain comprising a
(i) —OH group;
(ii) —(CH2)ₙOH group wherein n is 1, 2, or 3;
(iii) —(CH2)ₙCONH₂ group wherein n is 1, 2, or 3;
(iv) -a cyclic C₄H₉N group; or
(v) a branched alkylgroup.

The term “peroxidase” as used herein and interchangeably indicated throughout the art by the term “peroxide reductases” refers to enzymes of Enzyme Commission (E.C.) Class 1.11.1.x. It is appreciated by a skilled person that the term indicates a group of enzymes that catalyse the oxidation of a substrate by hydrogen peroxide or an organic peroxide. Examples of peroxidase enzymes suitable in the context of the present invention are peroxidase capable of oxidising one or more of a chloride ion, iodide ion, bromide or a thiocyanate ion to an antimicrobial hypochlorite, hypoiodite, hypobromite or hypothiocyanite ion, respectively. By means of illustration and not limitation, suitable peroxidase enzymes include lactoperoxidase, guaiacol peroxidase, plant peroxidase, Japanese radish peroxidase, horseradish peroxidase (HRP), soybean peroxidase (SBP), extensin peroxidase, heme peroxidase, oxyperoxidase, protoheme peroxidase, pyrocatechol peroxidase, scopoletin peroxidase, Coprinus cinereus peroxidase, and Arthromyces ramosus peroxidase.

Related to the above, the term “peroxidase activity” is defined herein as an enzymatic activity that converts a peroxide, e.g., hydrogen peroxide, to a less oxidative species, e.g., water. It is understood herein that a molecule having peroxidase activity encompasses a peroxide-decomposing enzyme. The term “peroxide” is well known in the art and indicates a group of compounds comprising a generally structure of R—O—O—R, wherein R can be any element. A hallmark example of a peroxide is hydrogen peroxide (H₂O₂), which is commonly indicated by the term “peroxide”.

Optionally, the peroxidase is a heme-peroxidase. Alternatively and equally optionally, the peroxidase is a non-heme peroxidase. In further optional embodiments, the composition described herein comprises at least one heme-peroxidase and at least on non-heme peroxidase.

A particularly preferred peroxidase in the context of the present invention is lactoperoxidase. Lactoperoxidase (abbreviated LPO) is a glycoprotein categorized as an oxidoreductase which naturally occurs in milk. Lactoperoxidase catalyses the production of hypothiocyanic acid and water from hydrogen peroxide (H₂O₂) and thiocyanic acid. The origin of the lactoperoxidase is not particularly limited and may therefore be derived from any suitable source such as plants, animals, microbes or any products derived thereof such as human milk, cow milk, horse milk, sheep milk, and goat milk. In such embodiments, the lactoperoxidase may be derived from milk of skim milk using any suitable method known to a skilled person for example ion chromatography (as described by e.g. Borzouee et al., Adv Biomed Res, 2016). Alternatively, the lactoperoxidase may be recombinantly produced (as described by e.g. Watanabe et al., FEBS Lett, 1998). Particularly preferred lactoperoxidases in the context of the present invention are human (UniProt ID P22079) and bovine lactoperoxidase (UniProt ID P80025) (which share the same amino acid sequence). A skilled person appreciates that when reference is made to human and/or bovine lactoperoxidase all human and/or bovine isoforms are equally envisaged. By means of illustration and not limitation, a suitable bovine lactoperoxidase is Bos taurus lactoperoxidase. Optionally, the lactoperoxidase is a monomeric glycoprotein having a molecular weight of 77,500 Da, an IEP of 9.6 and a pH optimum at pH 6. Alternatively, the lactoperoxidase may be a modified glycoprotein having a molecular weight of less than 77.5 Da, and/or an IEP that is not 9.6, and/or a pH optimum that is not at pH 6.

Lactoperoxidase having a substantial sequence identity to SEQ ID NO: 1 is particularly envisaged in the present context. However, since lactoperoxidase is initially translated as a protein comprising an additional propeptide and signal peptide, lactoperoxidases having a substantial sequence identity to SEQ ID NO: 2 and/or SEQ ID NO: 3 are equally envisaged. Hence, in preferred embodiments the lactoperoxidase comprises an amino acid sequence having a sequence identity of at least 75%, preferably at least 80%, preferably at least 85%, preferably at least 90%, preferably at least 95%, preferably at least 97.5%, or even 100% sequence identity to SEQ ID NO: 1.

The amino acid sequence of Bos taurus lactoperoxidase is herewith reproduced:

(SEQ ID NO: 1)
DTTLTNVTDPSLDLTALSWEVGCGAPVPLVKCDENSPYRTITGDCNNRRSPALGAANRALARWLPAEYEDGLALP
FGWTQRKTRNGFRVPLAREVSNKIVGYLDEEGVLDQNRSLLFMQWGQIVDHDLDFAPETELGSNEHSKTQCEEYC
IQGDNCFPIMFPKNDPKLKTQGKCMPFFRAGFVCPTPPYQSLAREQINAVTSFLDASLVYGSEPSLASRLRNLSS
PLGLMAVNQEAWDHGLAYLPFNNKKPSPCEFINTTARVPCFLAGDFRASEQILLATAHTLLLREHNRLARELKKL
NPHWNGEKLYQEARKILGAFIQIITFRDYLPIVLGSEMQKWIPPYQGYNNSVDPRISNVFTFAFRFGHMEVPSTV
SRLDENYQPWGPEAELPLHTLFFNTWRIIKDGGIDPLVRGLLAKKSKLMNQDKMVTSELRNKLFQPTHKIHGFDL
AAINLQRCRDHGMPGYNSWRGFCGLSQPKTLKGLQTVLKNKILAKKLMDLYKTPDNIDIWIGGNAEPMVERGRVG
PLLACLLGROFQQIRDGDRFWWENPGVFTEKQRDSLQKVSFSRLICDNTHITKVPLHAFQANNYPHDFVDCSTVD
KLDLSPWASREN.

In alternative preferred embodiments the lactoperoxidase comprises an amino acid sequence having a sequence identity of at least 75%, preferably at least 80%, preferably at least 85%, preferably at least 90%, preferably at least 95%, preferably at least 97.5%, or even 100% sequence identity to SEQ ID NO: 2.

The amino acid sequence of Bos taurus lactoperoxidase (+propeptide) is herewith reproduced:

(SEQ ID NO: 2)
DTIAQAASTTTISDAVSKVKIQVNKAFLDSRTRLKTTLSSEAPTTQQLSEYFKHAKGRTRTAIRNGQVWEESLKR
LRRDTTLTNVTDPSLDLTALSWEVGCGAPVPLVKCDENSPYRTITGDCNNRRSPALGAANRALARWLPAEYEDGL
ALPFGWTQRKTRNGFRVPLAREVSNKIVGYLDEEGVLDQNRSLLFMQWGQIVDHDLDFAPETELGSNEHSKTQCE
EYCIQGDNCFPIMFPKNDPKLKTQGKCMPFFRAGFVCPTPPYQSLAREQINAVTSFLDASLVYGSEPSLASRLRN
LSSPLGLMAVNQEAWDHGLAYLPFNNKKPSPCEFINTTARVPCFLAGDFRASEQILLATAHTLLLREHNRLAREL
KKLNPHWNGEKLYQEARKILGAFIQIITFRDYLPIVLGSEMQKWIPPYQGYNNSVDPRISNVFTFAFRFGHMEVP
STVSRLDENYQPWGPEAELPLHTLFFNTWRIIKDGGIDPLVRGLLAKKSKLMNQDKMVTSELRNKLFQPTHKIHG
FDLAAINLQRCRDHGMPGYNSWRGFCGLSQPKTLKGLQTVLKNKILAKKLMDLYKTPDNIDIWIGGNAEPMVERG
RVGPLLACLLGRQFQQIRDGDRFWWENPGVFTEKQRDSLQKVSFSRLICDNTHITKVPLHAFQANNYPHDFVDCS
TVDKLDLSPWASREN

In yet alternative preferred embodiments the lactoperoxidase comprises an amino acid sequence having a sequence identity of at least 75%, preferably at least 80%, preferably at least 85%, preferably at least 90%, preferably at least 95%, preferably at least 97.5%, or even 100% sequence identity to SEQ ID NO: 3.

The amino acid sequence of Bos taurus lactoperoxidase (+propeptide+signal peptide) is herewith reproduced:

(SEQ ID NO: 3)
MWVCLQLPVFLASVTLFEVAASDTIAQAASTTTISDAVSKVKIQVNKAFLDSRTRLKTTLSSEAPTTQQLSEYFK
HAKGRTRTAIRNGQVWEESLKRLRRDTTLTNVTDPSLDLTALSWEVGCGAPVPLVKCDENSPYRTITGDCNNRRS
PALGAANRALARWLPAEYEDGLALPFGWTQRKTRNGFRVPLAREVSNKIVGYLDEEGVLDQNRSLLFMQWGQIVD
HDLDFAPETELGSNEHSKTQCEEYCIQGDNCFPIMFPKNDPKLKTQGKCMPFFRAGFVCPTPPYQSLAREQINAV
TSFLDASLVYGSEPSLASRLRNLSSPLGLMAVNQEAWDHGLAYLPFNNKKPSPCEFINTTARVPCFLAGDFRASE
QILLATAHTLLLREHNRLARELKKLNPHWNGEKLYQEARKILGAFIQIITFRDYLPIVLGSEMQKWIPPYQGYNN
SVDPRISNVFTFAFRFGHMEVPSTVSRLDENYQPWGPEAELPLHTLFFNTWRIIKDGGIDPLVRGLLAKKSKLMN
QDKMVTSELRNKLFQPTHKIHGFDLAAINLQRCRDHGMPGYNSWRGFCGLSQPKTLKGLQTVLKNKILAKKLMDL
YKTPDNIDIWIGGNAEPMVERGRVGPLLACLLGRQFQQIRDGDRFWWENPGVFTEKQRDSLQKVSFSRLICDNTH
ITKVPLHAFQANNYPHDFVDCSTVDKLDLSPWASREN.

In yet alternative preferred embodiments the lactoperoxidase comprises three amino acid sequences that each independently have at least 75%, preferably at least 80%, preferably at least 85%, preferably at least 90%, preferably at least 95%, preferably at least 97.5%, or even 100% sequence identity to SEQ ID NO: 1, SEQ ID NO: 4, and SEQ ID NO: 5.

The isolated Bos taurus lactoperoxidase signal peptide is herewith reproduced:

(SEQ ID NO: 4)
MWVCLQLPVFLASVTLFEVAAS.

The isolated Bos taurus lactoperoxidase propeptide is herewith reproduced:

(SEQ ID NO: 5)
DTIAQAASTTTISDAVSKVKIQVNKAFLDSRTRLKTTLSSEAPTTQQLS
EYFKHAKGRTRTAIRNGQVWEESLKRLRR.

A skilled person appreciates that dependent on the particulars of the synthesis and/or purification method, a methionine residue is present at the N-terminus of the envisaged peroxidase.

“Peroxide generating system” as used herein refers to any molecule or substance capable of generating a peroxide. The peroxide generating system may be a single molecule, or a group of distinct molecules. Typically, the peroxide-generating system comprises a peroxide generating enzyme and a substrate. In certain embodiments, the peroxide generating enzyme is an oxidoreductase enzyme. Suitable oxidoreductases include without limitation glucose oxidase, galactose oxidase, glycollate oxidase, lactate oxidase, L-gulunolactone oxidase, L-2-hydroxyacid oxidase, aldehyde oxidase, xanthine oxidase, D-aspartate oxidase, L-amino acid oxidase, D-amino acid oxidase, monoamine oxidase, pyridoxaminephosphate oxidase, diamine oxidase, and sulphite oxidase.

Suitable substrates for use in the peroxide generating system according to the present invention include the natural substrates of the enzymes listed above, as well as other substrates, which allow the generation of peroxide. Beta-D-glucose is a specific substrate for glucose oxidase. Other suitable substrates include, but are not limited to D-glucose, 0-galactose, L-sorbose, ethanol, tyramine, 1,4-diaminobutane, 2-aminophenol, glycollate, L-lactate, 2-deoxy-D-glucose, L-gulunolactone, L-galaconolactone, D-mannonolactone, L-2-hydroxyisocaproate, acetaldehyde, butyraldehyde, xanthine, D-aspatate, D-glutamate, L-amino acids and D-amino acids.

Alternatively, the peroxide generating system is non-enzymatic and the hydrogen peroxide is generated in alternative ways, e.g. by molecules which naturally degrade generating peroxide, such as perborate or percarbonate salts, more particularly sodium percarbonate or sodium perborate.

A preferred peroxidase generating system in the context of the present invention is glucose oxidase (GOX), optionally combined with glucose as a substrate. Glucose oxidase is an enzyme that oxidizes β-D-glucose to produce D-glucono-δ-lactone and hydrogen peroxide. The glucose oxidase used in the context of the present invention is not particularly limited. Optionally, the glucose oxidase is obtained from a microorganism, such as but not limited to Aspergillus niger and Penicillium chrysogenum. Glucose oxidase derived from the microorganism may be obtained by any suitable method described in the art for producing a microorganism-derived enzyme. Further, a commercially available glucose oxidase may be used, or a commercially available glucose oxidase derived from a microorganism may be used.

As used herein a ‘halide’ refers to an ion of a halogen. Halides are binary chemical compounds, of which one part is a halogen atom and the other part is an element or radical that is less electronegative (or more electropositive) than the halogen to make a fluoride, chloride, bromide, iodide, astatide, or theoretically tennesside compound. The alkali metals combine directly with halogens under appropriate conditions forming halides of the general formula, MX (X=F, Cl, Br or I). By means of illustration and not limitation, suitable halides include ionic iodides, optionally under the form of water-soluble iodide salts such as an alkaline metal iodide salt, e.g. potassium iodide (Kl), sodium iodide (NaI), lithium iodide, ammonium iodide, or calcium iodide. Typical examples are sodium iodide and potassium iodide. Particularly potassium iodide was unexpectedly found to further improve the antimicrobial properties of the compositions described throughout the present specification when compared to other halides such as potassium chloride and/or potassium bromide.

The term “pseudohalide” refers to a polyatomic anion resembling the halides in their acid-base and redox chemistry. These include cyanide, thiocyanate, thiosulfate and azide ions. Suitable sources of the thiocyanate ion (SCN—) include sodium thiocyanate, potassium thiocyanate, ammonium thiocyanate, and other thiocyanate salts. Typical examples are sodium thiocyanate and potassium thiocyanate. According to a particular embodiment the compositions of the present invention comprise lactoperoxidase and both a thiocyanate and a halide as electron donors.

The term “enhancing agents” is to be interpreted broadly and indicated any function that enhances peroxidase activity, and/or peroxidase-based anti-microbial activity, the enhancing agents envisaged in the context of the present invention are non-toxic compounds and/or compounds which are recognised as GRAS compounds (Generally Accepted As Save).

The enhancing agents in the context of the present invention are preferably hydrophilic compounds (i.e. water soluble), such as hydrophilic amino acids, hydrophilic amino acid mimics, or hydrophilic amino acid derivatives. A skilled person appreciates that the term “hydrophilic compound” or “hydrophilic molecule” refers to a molecule whose interactions with water and other polar substances are more thermodynamically favourable than their interactions with oil or other hydrophobic solvents. Hydrophilic molecules are typically charge-polarized and able to engage in hydrogen bonding.

The term “amino acid” indicates naturally occurring amino acids, naturally encoded amino acids, non-naturally encoded amino acids, non-naturally occurring amino acids, amino acid analogues and amino acid mimetics that function in a manner similar to the naturally occurring amino acids, all in their D- and L-stereoisomers, provided their structure allows such stereoisomeric forms. Amino acids are referred to herein with their full name, their three-letter abbreviation or their one letter abbreviation. Amino acids are referred to herein by either their name, their commonly known three letter symbols or by the one-letter symbols recommended by the IUPAC-IUB Biochemical Nomenclature Commission. A “naturally encoded amino acid” indicates an amino acid that is one of the 20 common amino acids or pyrrolysine, pyrroline-carboxy-lysine or selenocysteine. The 20 common amino acids are: alanine (A or Ala), cysteine (C or Cys), aspartic acid (D or Asp), glutamic acid (E or Glu), phenylalanine (F or Phe), glycine (G or Gly), histidine (H or His), isoleucine (I or Ile), lysine (K or Lys), leucine (L or Leu), methionine (M or Met), asparagine (N or Asn), proline (P or Pro), glutamine (Q or Gln), arginine (R or Arg), serine (S or Ser), threonine (T or Thr), valine (V or Val), tryptophan (W or Trp), and tyrosine (Y or Tyr). Also envisaged by said term are amino acid analogues, wherein at least one individual atom is replaced either with a different atom, an isotope of the same atom, or with a different functional group.

“Amino acid mimics” as used herein indicates any molecule that has the same functional properties as the amino acid “that is being mimicked”. Hence, the general chemical formula of an amino acid mimic may divert from the typical R—CH(NH₂)—COOH formula and may by means of illustration and not limitation have a formula according to R—CH(NH₂)(CH2)ₙ—COOH, wherein n is any integer. A skilled person appreciates that the term “amino acid mimics” does not encompass aminoalcohols. Particularly, a skilled person appreciates that the term “amino acid mimics” does not encompass 1, 2 aminoalcohols such as ethanolamine which contains both a primary alcohol and a primary amine.

“Amino acid derivatives” as used herein refer to molecules that comprise, or comprise substantially the same chemical formula as an amino acid, wherein one or more functional groups are modified, added, or deleted. The term therefore encompasses any derivative of an amino acid resulting from reaction at an amino group, carboxy group, side-chain functional group, or from the replacement of any hydrogen by a heteroatom.

In certain embodiments, the enhancing agent is a hydrophilic amino acid, amino acid mimic, or amino acid derivative, characterized by a free amino functional group (—NH₃⁺), free carboxylic acid functional group (—CO₂H), and a side chain comprising an OH group. In alternative embodiments, the enhancing agent is a hydrophilic amino acid, amino acid mimic, or amino acid derivative, characterized by a free amino functional group (—NH₃⁺), free carboxylic acid functional group (—CO₂H), and a side chain comprising a —(CH₂)ₙOH group wherein n is 1, 2, or 3. In yet alternative embodiments, the enhancing agent is a hydrophilic amino acid, amino acid mimic, or amino acid derivative, characterized by a free amino functional group (—NH₃⁺), free carboxylic acid functional group (—CO₂H), and a side chain comprising a —(CH₂)ₙCONH₂ group wherein n is 1, 2, or 3. In yet alternative embodiments, the enhancing agent is a hydrophilic amino acid, amino acid mimic, or amino acid derivative, characterized by a free amino functional group (—NH₃⁺), free carboxylic acid functional group (—CO₂H), and a side chain comprising a cyclic C₄H₉N group. In yet alternative embodiments, the enhancing agent is a hydrophilic amino acid, amino acid mimic, or amino acid derivative, characterized by a free amino functional group (—NH₃⁺), free carboxylic acid functional group (—CO₂H), and a side chain comprising a branched alkylgroup.

In certain embodiments, the enhancing agent is an organic compound characterised by the presence of an amino and carboxylate functional group and further comprising a functional group selected from the group consisting of: polar uncharged methanol, ethan-1-ol, acetamide, propenamide, or a secondary butyl group; and a connection between the amino and carboxylate functional group and a side chain that consists of (CH₂)ₙ where n=1, 2 or 3. In certain embodiments, the enhancing agent is an organic compound characterised by the presence of a pyrrolidine and carboxylate functional group; and a connection between the amino and carboxylate functional group and a side chains that consist of (CH₂)ₙ where n=1, 2 or 3.

The term “pyrrolidine”, interchangeably indicated throughout the art with the term “tetrahydropyrrole” is known to a skilled person and refers to an organic compound characterised by the molecular formula (CH₂)₄NH. Pyrrolidine is a cyclic secondary amine alternatively classified as a saturated heterocycle.

In certain embodiments, the enhancing agent is a hydrophilic amino acid, amino acid mimic, or amino acid derivative having an octanol/water partition coefficient (Log P) of from about −1 to about −5. In further embodiments, the enhancing agent is a naturally occurring amino acid having an octanol/water partition coefficient (Log P) of from about −1.25 to about −4.5. In yet further embodiments, the enhancing agent is a naturally occurring amino acid having an octanol/water partition coefficient (Log P) of from about −1.5 to about −4. In even further embodiments, the enhancing agent is a naturally occurring amino acid having an octanol/water partition coefficient (Log P) of from about −1.72 to about −3.82.

The (n-)octanol/water partition coefficient, (alternatively indicated in the art by “partition coefficient” and abbreviated as “K_{ow}”) is a partition coefficient for the two-phase system consisting of n-octanol and water. K_{ow} is also frequently referred to by the symbol P, and commonly expressed as a common logarithm (i.e. Log P). It is also called n-octanol-water partition ratio. The partition coefficient of a molecule indicates the lipophilicity (fat solubility) and hydrophilicity (water solubility) of a molecule. Hydrophobicity of a given molecule is indicated by higher partition coefficient values, while hydrophilicity of a given molecule is indicated by lower partition coefficient values. Exemplary partition coefficient values for a non-exhaustive list of envisaged amino acid suitable to act as an enhancing agent in the context of the present invention are given below:

| | |
|---|---|
| Serine | LogP = −3.07 |
| Threonine | LogP = −2.94 |
| Asparagine | LogP = −3.82 |
| Glutamine | LogP = −3.15 |
| Isoleucine | LogP = −1.72 |
| Proline | LogP = −2.54 |

In certain embodiments, the enhancing agent is a hydrophilic naturally or non-naturally occurring amino acid. In further embodiments, the enhancing agent is a naturally occurring amino acid selected from the group consisting of serine, threonine, asparagine, glutamine, isoleucine, proline, or any combination thereof.

In certain embodiments, the enhancing agent is present in the composition at a concentration of from about 0.0005 mg/ml to about 200 mg/ml, preferably of from about 0.005 mg/ml to about 10 mg/ml, most preferably of from about 0.05 to about 1 mg/ml. In certain embodiments, the enhancing agent is present in the composition at a concentration of at least about 0.0001 mg/ml, preferably at a concentration of at least about 0.005 mg/ml, most preferably at a concentration of at least about 0.05 mg/ml.

In preferred embodiments, the enhancing agent is a naturally occurring amino acid selected from the group consisting of serine, threonine, asparagine, glutamine, isoleucine, proline, or any combination thereof and the enhancing agent is present in the composition at a concentration of from about 0.0005 mg/ml to about 200 mg/ml, preferably of from about 0.005 mg/ml to about 10 mg/ml, most preferably of from about 0.05 to about 1 mg/ml.

Optionally, the composition comprises an enhancing agent that is a naturally occurring amino acid selected from the group consisting of serine, threonine, asparagine, glutamine, isoleucine, proline, or any combination thereof and a water-soluble iodide salt. In preferred embodiments, the composition comprises an enhancing agent is a naturally occurring amino acid selected from the group consisting of serine, threonine, asparagine, glutamine, isoleucine, proline, or any combination thereof, lactoperoxidase, a water-soluble iodide salt, and glucose oxidase.

Optionally, the composition comprises an enhancing agent that is a naturally occurring amino acid selected from the group consisting of serine, threonine, asparagine, glutamine, isoleucine, proline, or any combination thereof, a peroxidase such as lactoperoxidase, a halide or pseudohalide such as a water-soluble iodide salt (for example potassium iodide, KI), a peroxide generating system such as glucose oxidase, and a further enhancing agent that is a benzene molecule substituted with a —OH or a (CH₂)ₙOH (n=1, 2, 3 or 4) group and substituted with one alkoxy group (—OR) with a chain length of 1, 2, 3 or 4 carbon atoms.

Optionally, the composition comprises an enhancing agent that is a naturally occurring amino acid selected from the group consisting of serine, threonine, asparagine, glutamine, isoleucine, proline, or any combination thereof, lactoperoxidase, a water-soluble iodide salt, glucose oxidase, and a further enhancing agent that is guaiacol, vanillin, or a combination thereof.

“Guaiacol” has been described in the art and indicates an organic compound characterised by the chemical formula C₆H₄(OH)(OCH₃). The origin of the guaiacol optionally used in the context of the present invention is not particularly limited, and may for example be obtained from guaiacum, wood creosote, or essential oils of celery seeds, tobacco leaves, orange leaves, and lemon peels. Suitable means to chemically synthesize guaiacol include without limitation hydrolysis of o-anisidine by its diazonium derivative, or by the demethylation of catechol followed by selective mono-demethylation. The phenolic aldehyde “vanillin” as described herein may be derived from guaiacol or lignin, and is characterised by the chemical formula C₈H₈O₃. The origin of the vanillin in the context of the present disclosure is not particularly limiting and therefore vanillin may equally be obtained by biosynthesis through V. planifolia, chemical synthesis from eugenol, or by means of genetically modified microorganisms.

The terms “formulation” or “composition” may be used interchangeably herein. In any of the embodiments concerning one or more of the compositions described herein, it is evident that said composition may comprise one or more pharmaceutically or cosmetically acceptable carriers (i.e. excipients). The term “pharmaceutically acceptable” or “cosmetically acceptable” as used herein is consistent with the art and means compatible with the other ingredients of a pharmaceutical or cosmetic composition and not deleterious to the recipient thereof.

In any of the embodiments described herein, the composition may comprise one or more excipients. The term “excipient” as used interchangeably herein and in the art with “carrier” may be indicative for any solvent, diluent, buffer (including but not limited to neutral buffered saline, phosphate buffered saline, or optionally Tris-HCl, acetate or phosphate buffers), solubilizer (including but not limited to Tween 80 or Polysorbate 80), colloid, dispersion medium, vehicle, filler, chelating agent (including but not limited to EDTA or glutathione), amino acid, protein, disintegrant, binder, lubricant, wetting agent, stabilizer, emulsifier, sweetener, colorant, flavoring, aromatizer, thickener, any agent suitable to achieve a depot effect, coating, antifungal agent, any preservative (including but not limited to benzalkonium chloride, or benzyl alcohol), antioxidant (including but not limited to ascorbic acid, sodium metabisulfite), tonicity controlling agent, absorption delaying agent, adjuvant, bulking agent (including but not limited to lactose, mannitol) and any other ingredient that may influence any parameter or characteristic of the composition, or of the polypeptide comprised in the composition described herein. A skilled person understands that one or more excipients may be used in the composition on condition that the one or more excipient is compatible with the one or more pharmaceutical ingredient (i.e. in the context of the present invention at least the polypeptide comprising the variant amino acid sequence) and that a pharmaceutically acceptable formulation is obtained.

In certain embodiments, the excipient may be an active pharmaceutical ingredient excipient, binder excipient, carrier excipient, co-processed excipient, coating system excipient, controlled release excipient, diluent excipient, disintegrant excipient, dry powder inhalation excipient, effervescent system excipient, emulsifier excipient, lipid excipient, lubricant excipient, modified release excipient, penetration enhancer excipient, permeation enhancer excipient, pH modifier excipient, plasticizer excipient, preservative excipient, preservative excipient, solubilizer excipient, solvent excipient, sustained release excipient, sweetener excipient, taste making excipient, thickener excipient, viscosity modifier excipient, filler excipient, compaction excipient, dry granulation excipient, hot melt extrusion excipient, wet granulation excipient, rapid release agent excipient, increased bioavailability excipient, dispersion excipient, solubility enhancement excipient, stabilizer excipient, capsule filling excipient, or any combination hereof. A skilled person is aware that use of such media and agents for pharmaceutical active substances is common practice and incorporation of these excipients is hence well known in the art. It is evident that all of the used ingredients should be non-toxic in the concentration contained in the final composition and should not negatively interfere with the activity of the one or more pharmaceutically active ingredients, in the present context at least the peroxide generating system and the peroxidase. In certain embodiments, more than one excipient which a skilled person would classify as belonging to the same group of excipients is added to the composition. In further embodiments, more than one excipient wherein the different excipients belong to different groups is added to the composition. In certain embodiments, the excipients may fulfill more than one function and/or be classified by a skilled person as belonging to different groups or classes of excipients.

Furthermore, the composition may comprise pharmaceutically acceptable auxiliary substances as required to approximate physiological conditions, such as pH adjusting and buffering agents, preservatives, complexing agents, tonicity adjusting agents, wetting agents and the like, non-limiting examples include sodium acetate, sodium lactate, sodium phosphate, sodium hydroxide, hydrogen chloride, benzyl alcohol, parabens, EDTA, sodium oleate, sodium chloride, potassium chloride, calcium chloride, sorbitan monolaurate, and triethanolamine oleate. In certain embodiments, at least one additional component is combined with the composition prior to administration. In further embodiments, the additional component is combined with the composition immediately prior to administration. In certain embodiments, the amount of the additional component added to the composition is calculated based on certain patient parameters including but not limited to age, weight, gender, severity of the disease condition, and other known disease conditions of the patient or disease conditions the patient is suspected to be afflicted with.

According to one embodiment of the invention, the compositions of the present invention comprise both a peroxidase enzyme and an electron donor for the peroxidase. Typically, when lactoperoxidase is used, iodide and/or thiocyanate ions are added as donors for the lactoperoxidase enzyme. According to an alternative embodiment the enhancing agents of the present invention partially or completely replaces the donor molecule. This has as an additional advantage that toxic compounds such as thiocyanates can fully or partially be replaced by less toxic or non-toxic compounds.

Thus, in a particular embodiment, the antimicrobial and anti-inflammatory active compound in the compositions of the present invention consist of a peroxide or a peroxide generating system, a lactoperoxidase, a halide and an enhancing agent selected from the group consisting of: serine, threonine, asparagine, glutamine, isoleucine, proline, or any combination thereof. In a further particular embodiment, the antimicrobial and anti-inflammatory active compound in the compositions of the present invention consist of a peroxide or a peroxide generating system, a lactoperoxidase, iodide anions and enhancing agent selected from the group consisting of: serine, threonine, asparagine, glutamine, isoleucine, proline, or any combination thereof.

The enhancing agents described herein increase the activity of the peroxide generating system and/or the peroxidase. The enhancing agents increase the activity of the peroxide generating system and/or the peroxidase by at least about 5%, preferably at least about 10%, more preferably by at least about 20%, more preferably by at least about 30%, more preferably by at least about 40%, more preferably by at least about 50%, more preferably by at least about 60%, more preferably by at least about 70%, more preferably by at least about 80%, more preferably by at least about 90%, most preferably by at least about 100%.

Optionally, the enhancing agents increase the activity of the peroxide generating system and/or the peroxidase by at least 1.2 fold, preferably at least 1.5 fold, preferably at least 1.75 fold, preferably at least 2 fold, more preferably at least 2.5 fold, most preferably at least 5 fold.

A skilled person appreciates that the relative increases in the activity of the peroxide generating system and/or the peroxidase as described herein should be interpreted with respect to a reference activity in a suitable control condition, i.e. a composition that does not comprise as enhancing agent as described herein.

A suitable control composition is therefore a composition comprising a peroxide generating system, a peroxidase, a halide or pseudohalide that are identical to the composition subject of investigation with regards to activity. An alternative suitable control composition is therefore a composition comprising a peroxide generating system, a peroxidase, a halide or pseudohalide that are identical to the composition subject of investigation with regards to activity, whereto an appropriate amount of enhancing agent subject of WO 2006/133523 is added. Thus, suitable control enhancing agents for use in control compositions include benzene molecules substituted with a —OH or a (CH₂)ₙOH (n=1, 2, 3 or 4) group and substituted with one alkoxy group (—OR) with a chain length of 1, 2, 3 or 4 carbon atoms. Optionally, the control enhancing agent is guaiacol or vanillin.

The compositions subject of the invention are characterised by a potent anti-microbial activity, which is further increased by the enhancing agents described herein. “Antimicrobial” refers to killing microbes or retarding the growth of microbes. Microbes include bacteria such as gram-negative bacteria (e.g. Escherichia coli and Pseudomonas aeruginosa), gram-positive bacteria (e.g. Staphylococcus aureus, Propionibacterium acnes) and spore-forming bacteria. Microbes include fungi such as moulds (e.g. Aspergillus niger, Penicillium funiculosum), yeasts (e.g. Candida albicans, Saccharomyces cerevisiae, Pityrosporum ovale) and dermatophytic fungi (e.g. Trichophyton rubrum). The term antimicrobial thus comprises the terms “bactericidal”, “bacteriostatic”, “fungicidal” and “fungistatic”. Microbes may also include microalgae such as Chlorella spp. and Spyrogyra spp. and viruses such as Herpes virus, Picornavirus, Varicella and warts.

Optionally, the composition is characterised by a Minimum Inhibitory Concentration (MIC) and/or a Minimal Bactericidal Concentration (MBC) and/or absolute counts and/or enzymatic activity for an inoculum of S. aureus, P. aeruginosa, C. albicans, A. niger, or any combination thereof that is significantly reduced when compared to a control composition as described herein. The expression “Minimum Inhibitory Concentration” is known to a skilled person and is to be interpreted according to the commonly accepted interpretation in the art. Therefore, the minimum inhibitory concentration as referred to herein indicates the lowest concentration of an antibacterial composition to inhibit growth of a particular bacterium or group of distinct bacteria. In certain embodiments, the minimum inhibitory concentration and/or a minimal bactericidal concentration and/or absolute counts and/or enzymatic activity for an inoculum of S. aureus, P. aeruginosa, C. albicans, A. niger, or any combination thereof is reduced by at least about 5%, preferably by at least about 10%, preferably by at least about 15%, preferably by at least about 20%, preferably by at least about 25%, preferably by at least about 30%, preferably by at least about 40%, preferably by at least about 50%, preferably by at least about 60%, more preferably by at least about 70%, most preferably by at least about 75% when compared to a control composition.

Optionally, the composition is characterised by a Minimal Bactericidal Concentration (MBC) for an inoculum of S. aureus, P. aeruginosa, C. albicans, A. niger, or any combination thereof that is significantly reduced when compared to a control composition as described herein. The expression “Minimal Bactericidal Concentration” is known to a skilled person and is to be interpreted according to the commonly accepted interpretation in the art. Therefore, the minimal bactericidal concentration as referred to herein indicates the lowest concentration of an antibacterial composition to kill a particular bacterium or group of distinct bacteria. In certain embodiments, the minimal bactericidal concentration for an inoculum of S. aureus, P. aeruginosa, C. albicans, A. niger, or any combination thereof is reduced by at least about 5%, preferably by at least about 10%, preferably by at least about 15%, preferably by at least about 20%, preferably by at least about 25%, preferably by at least about 30%, preferably by at least about 40%, preferably by at least about 50%, preferably by at least about 60%, more preferably by at least about 70%, most preferably by at least about 75% when compared to a control composition as described herein.

Optionally, both the minimum inhibitory concentration and the minimal bactericidal concentration of the composition described herein are reduced by an amount of from about 10% to about 75%, preferably of from about 20% to about 65%, more preferably of from about 30% to about 50% when compared to a control composition. Methods to determine the minimum inhibitory concentration and/or minimal bactericidal concentration of a composition have been described in the art at numerous occasions and are therefore known to a skilled person. Preferably, the minimum inhibitory concentration and/or minimal bactericidal concentration is determined by incubation of said composition for 24 hours at 37° C. for S. aureus, P. aeruginosa, C. albicans and 48 hours at 20-25° C. for A. niger in a microbial inoculum characterised by an OD600 of 0.1. Optionally, the minimum inhibitory concentration and/or minimal bactericidal concentration is reduced by the extent described above when compared to a control composition comprising a peroxide generating system, a peroxidase, a halide or pseudohalide that are identical to the composition subject of investigation with regards to activity. An alternative suitable control composition is therefore a composition comprising a peroxide generating system, a peroxidase, a halide or pseudohalide that are identical to the composition subject of investigation with regards to activity, and optionally whereto an appropriate amount of enhancing agent subject of WO 2006/133523 is added. Thus, suitable control enhancing agents for use in control compositions include benzene molecules substituted with a —OH or a (CH₂)ₙOH (n=1, 2, 3 or 4) group and substituted with one alkoxy group (—OR) with a chain length of 1, 2, 3 or 4 carbon atoms. Optionally, the control enhancing agent is guaiacol or vanillin.

Preferably, the compositions described herein are preferably topical compositions. It is appreciated that topical compositions are formulated in such a manner to render them particularly useful for localized administration (i.e. application) of the composition described herein to a discrete surface of subject tissue. Related hereto, “cutaneous application” indicates administration to the skin of a subject. Topical administration also may involve the use of transdermal administration means such as but not limited to transdermal patches, wound dressings, or any impregnated material as discussed further below. Optionally, the composition is a topical therapeutic composition for cutaneous application. Alternatively, the composition is a topical prophylactic composition for cutaneous application. Yet alternatively, the composition is a topical cosmetic composition for cutaneous application.

The terms “subject”, “individual” or “patient” can be used interchangeably herein, and typically and preferably denote humans, but may also encompass reference to non-human animals, preferably warm-blooded animals, even more preferably mammals, such as, e.g., non-human primates, rodents, canines, felines, equines, ovines, porcines, and the like. The term “non-human animals” includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In certain embodiments, the subject is a non-human mammal. Preferred subjects are human subjects including all genders and all age categories thereof. Both adult subjects, new-born subjects, and foetuses are intended to be covered by the term “subject”. Thus, both adult and new-born subjects are intended to be covered. Examples of subjects include humans, dogs, cats, cows, goats, and mice. Preferred subjects in the context of the invention are human subjects.

Optionally, the composition is a cream, lotion, foundation, ointment, suspension (oil-in-water and water-in-oil), (hydro)gel, foam, emulsion, paste, paint, spray, oxymel, liniment, insufflation, patch, or dressing.

The term “emulsion” indicates any mixture of at least two liquids that are unmixable (i.e., immiscible, unblendable) and therefore mixtures wherein a first liquid is distributed in small droplets (dispersed phase) throughout a second liquid (dispersion medium). Therefore, the composition may be an oil-in-water or water-in-oil emulsion. Emulsions are widely used in skin care formulations. Related hereto, “suspension” broadly refers to a heterogeneous mixture containing solids dispersed in a liquid phase that are not dissolved and have a size which is sufficiently large to allow for sedimentation.

“Cream” generally refers to a water-in-oil emulsion wherein an aqueous phase is dispersed in an oil phase, but may equally be an oil-in-water emulsion in which an oil is dispersed within an aqueous base. It is generally accepted that creams differ from emulsions in that emulsions are stable suspensions of small immiscible droplets of fluid immiscible with another fluid part of the emulsion, while a cream indicates a particular subset of emulsions that are more viscous and usually include more lipophilic and/or surfactant components.

“Lotions” are low- to medium-viscosity liquid compositions. Generally, lotions are less viscous than creams, however the viscosity of both may be similar. A lotion optionally finely powdered substances that are insoluble in the dispersion medium through the use of suspending agents and dispersing agents. Alternatively, lotions can have as the dispersed phase liquid substances that are immiscible with the vehicle and are usually dispersed by means of emulsifying agents or other suitable stabilizers. In one embodiment, the lotion is in the form of an emulsion having a viscosity of between 100 and 1000 centistokes. The fluidity of lotions permits rapid and uniform application over a wide surface area. Lotions are typically intended to dry on the skin leaving a thin coat of their components on the surface of the skin of the subject.

An “ointment” typically refers to a more viscous oil-in-water cream, i.e., to a semi-solid substance containing an ointment base and optionally one or more pharmaceutically active ingredients (in the context of the present invention a peroxidase and peroxide generating system). Examples of suitable ointment bases include hydrocarbon bases, absorption bases, water-removable bases, and water-soluble bases.

“Pastes” generally differ from ointments in that they contain a larger percentage of solids. Overall, pastes are more absorptive and less greasy when compared to ointments of a substantially similar composition. The base may be anhydrous (e.g. liquid or soft paraffin) or water soluble (e.g. glycerol or a mucilage).

“(Hydro)gels” are aqueous colloidal suspensions of a colloid in which particles are in the external or dispersion phase and water is in the internal or dispersed phase. Different components suitable for the manufacture of pharmaceutically acceptable hydrogels are known to the skilled person. Hydrogels are generally used for retaining or absorbing moisture or water. Suitable hydrogels in the context of the present invention are prepared with hydrocolloids such as alginates and polyacrylates (e.g. carbopol) and cellulose and derivatives thereof such as carboxymethyl cellulose (CMC). Other suitable hydrocolloids are aluminium hydroxide, siliciumdioxide or silicium acid, starch, glycogen, gelatin, pectin, chitosan, chitin, gum Arabic, locust bean gum, karaya gum, gum tragacanth, ghatti gum, agar-agar, carrageenans, carob gum, guar gum, xanthan gum, glyceryl polymethacrylate. A hydrogel can be used as such. Hydrogels for uses envisaged by the present disclosure can be formulated with different concentrations of colloid depending on the desired consistency.

“Foam” as used herein refers to a dispersion of gas particles in a liquid medium. Oil-in-water emulsions, water-in-oil emulsions, ethanol, water, solvents, liquid oil, propylene glycol, and glycerine can be listed as examples of liquid media in a foam. It is appreciated by a skilled person that foams may be generated by reducing the surface tension of the liquid mixing in (a) gaseous substance(s), causing bubble formulation. The acceptability of the foams is due to the fact that they are easy to apply on large areas of the skin, does not leave an oily or greasy film, and have rapid absorption into the skin.

“Mousse” refers to a substance closely resembling a foam, but is commonly used to denote substances that are less aqueous.

“Sprays” are preparations of drugs in aqueous, alcoholic or glycerin containing media. They are applied to the mucosae or (broken) skin with an atomiser or nebuliser.

“Paints” are liquids for application to the skin or mucosae, usually with a soft brush. Skin paints often have a volatile solvent that evaporates quickly to leave a dry or resinous film of medicament. Paints can be made viscous by the addition of glycerin, which is sticky, adheres to the affected site and prolongs the action of the drug.

Oxymels are preparations in which the vehicle is a mixture of an acid and honey.

“Liniments” are fluid, semi-fluid or, occasionally, semi-solid preparations intended for application to the skin. They may be alcoholic or oily solutions of emulsions. Most are massaged into the skin but some are applied on a dressing or with a brush.

“Insufflations” are medicated dusting powders that are blown by an insufflator (a device similar to an atomizer or pressurised atomizer).

Optionally, the composition is a deodorant, preferably a roll-on or stick deodorant. The term “deodorant”, or short “deo” refers to a cosmetic product that is applied to the skin that does not in itself prevent sweating but will cover up the smell of sweat by applying a stronger (more pleasant) scent or will prevent the development of sweat odour. Accordingly, the composition or formulation as described herein may be regarded as a product for promoting the personal hygiene of a subject. The term “antiperspirant” is a means that prevents sweating (for example by reducing or stopping sweating) so that there will also be less sweat odour. However, in today's cosmetic products the term “deodorant” refers to both “deodorant” and “antiperspirant”, and most deodorants are a combination of both.

Optionally, the composition is an anti-acne preparation, preferably in the form of a lotion or a cream. Acne vulgaris (referred to herein and in the art by the common name “acne”), is a skin disease that typically, although not exclusively, affects adolescents. The medical condition has been described at numerous occasions throughout the art and is therefore known to a skilled person. In brief, acne is caused by an obstruction of skin hair follicles by dead skin cells or oil produced by the skin. Acne is typically characterised by the presence of blackheads (open comedones), whiteheads (closed comedones), pimples, an oily appearance of the skin, and optionally scarring of the skin.

Optionally, the composition is an ear drop formulation, nose drop formulation, inhalation formulation, vaginal formulation, or rectal solution.

Optionally, the composition is comprised in and/or applied to an impregnated material such as wound dressings. In preferred embodiments, the wound dressing is a dressing configured for wound irrigation and/or treatment of wounds. Numerous types of dressing have been described in the art and include without limitation gauze dressings, tulle dressings, alginate dressings, polyurethane dressings, film dressings, polysaccharide paste dressings, granule dressings, foam dressings, silicone dressings, synthetic polymer scaffold dressings, hydrocolloid dressings, occlusive dressings or combinations thereof. The dressing may be adhesive or non-adhesive. The term “occlusive dressing” as used herein refers to a dressing that prevents air and/or bacteria from contacting the wound and contains one or more of the following: moisture, heat, body fluids, and medication. A skilled person is capable of selecting a suitable wound healing dressing to be used on a particular wound, and said selection may be made in function of parameters such as but not limited to the type of the wound, size of the wound, and healing progression of the wound. Optionally, the dressing is a hydrogel dressing. Hydrogel dressings are composed to a large extent of water in a network of fibres that maintain integrity of the polymer gel. Water from said dressing is released to preserve an adequate moisture level of the wound. Examples of hydrogel dressings include without limitation Tegagel® and Intrasite®.

Methods and protocols to produce any of the above dressings have been described in the art. The composition subject of the present disclosure may be incorporated into/onto the dressing upon manufacturing of said dressing, but may equally be applied to a premanufactured dressing. By means of illustration and not limitation, a premanufactured dressing or portion thereof may be impregnated with the composition described herein. Alternatively, a premanufactured dressing or portion thereof may be coated with the composition described herein.

In particular embodiments, the composition is comprised in a skin replacement (i.e. a skin substitute or a dermal substitute). A skin substitute provides a three dimensional biomatrix that fulfil the functions of a cutaneous dermal layer that is able to either temporarily or permanently cover open skin wounds. The material of said skin substitute is not particularly limited, and may therefore comprise of biological materials, synthetic material, or combinations thereof. Non-limiting examples of biological material include without limitation human or porcine skin, and human or porcine intestine submucosa. The biological skin substitute may comprise different constituents including but not limited to collagen, glycosaminoglycan, fibronectin, hyaluronic acid, elastin, and any combinations thereof.

Typically, the combination of a peroxide generating system, peroxidase and enhancing agent optionally combined with one or more halides and/or pseudo-halides are present in the pharmaceutical composition of the invention as active antimicrobial and anti-inflammatory ingredients.

The composition may comprise of from about 10 to 100000 U/kg, preferably from about 10 to 4000 U/kg, more preferably from about 10 to 100 U/kg of the peroxidase. In yet further preferred embodiments, the composition comprises of from about 10 to about 100 U/kg lactoperoxidase.

The composition may comprise of from about 0.0005 mg/ml to about 200 mg/ml, preferably of from about 0.005 mg/ml to about 10 mg/ml, (more preferably of from about 0.05 to about 1 mg/ml. In certain embodiments, the enhancing agent is present in the composition at a concentration of at least about 0.0001 mg/ml, preferably at a concentration of at least about 0.005 mg/ml, more preferably at a concentration of at least about 0.05 mg/ml, more preferably at a concentration of at least about 0.01 mg/ml, most preferably at a concentration of at least about 0.1 mg/ml, such as an enhancing agent characterised by a free amino functional group (—NH₃+), free carboxylic acid functional group (—CO₂H), and a side chain comprising a
(i) —OH group;
(ii) —(CH₂)ₙOH group wherein n is 1, 2, or 3;
(iii) —(CH₂)ₙCONH₂ group wherein n is 1, 2, or 3;
(iv) -a cyclic C₄H₉N group; or
(v) a branched alkylgroup.

In yet even further preferred embodiments, the composition comprises of from about 0.01 to about 100 mg/ml of an enhancing agent selected from the group consisting of: serine, threonine, asparagine, glutamine, isoleucine, proline, or any combination thereof.

The composition may comprise of from about 0.01 to about 500 mg/kg of one or more halides and/or pseudohalides. In preferred embodiments, the composition comprises of from about 0.1 to about 200 mg/kg, more preferably from about 1 mg to about 100 mg/kg, yet more preferably from about 2 mg to about 75 mg/kg, most preferably from 5 to 50 mg/kg of one or more halides and/or pseudohalides. In further preferred embodiments, the composition comprises of from about 0.1 to about 200 mg/kg of one or more water-soluble iodide salt, optionally wherein the one or more water-soluble iodide salt is potassium iodide.

The composition may comprise of from about 50 to about 10000 U/kg of peroxide generating enzyme (i.e. the enzymatic portion of the peroxide generating system). In preferred embodiments, the composition comprises of from about 100 to about 5000 U/kg of peroxide generating enzyme, more preferably of from about 150 to about 4000 U/kg, more preferably of from about 200 to about 3000 U/kg, more preferably of from about 300 to about 2500 U/kg of peroxide generating enzyme. In yet further preferred embodiments, the composition comprises of from about 300 to about 2500 U/kg of glucose oxidase.

In particular embodiments the composition comprises:
from about 150 to about 4000 U/kg, preferably of from about 200 to about 3000 U/kg, more preferably from about 300 to about 2500 U/kg of the peroxide generating system;
from about 10 to about 100000 U/kg, preferably from about 10 to 4000 U/kg, more preferably from about 10 to 100 U/kg of the peroxidase;
from about 0.01 to about 500 mg/kg, preferably from about 0.1 to about 200 mg/kg, more preferably from about 1 mg to about 100 mg/kg, yet more preferably from about 2 mg to about 75 mg/kg, most preferably from 5 to 50 mg/kg of the halide and/or pseudohalide;
and from 0.0005 mg/ml to 200 mg/ml, preferably of from 0.005 mg/ml to 10 mg/ml, most preferably of from 0.05 to 1 mg/ml of an enhancing agent, as described herein, such as but not limited to an enhancing agent selected from hydrophilic amino acid, amino acid mimic, or amino acid derivative, characterized by a free amino functional group (—NH₃+), free carboxylic acid functional group (—CO₂H), and a side chain comprising an
   (i) —OH group;
   (ii) —(CH₂)ₙOH group wherein n is 1, 2, or 3;
   (iii) —(CH₂)ₙCONH₂ group wherein n is 1, 2, or 3;
   (iv) -a cyclic C₄H₉N group; or
   (v) a branched alkylgroup.

In further embodiments the composition comprises:
from about 150 to about 4000 U/kg, preferably of from about 200 to about 3000 U/kg, more preferably from about 300 to about 2500 U/kg of glucose oxidase;
from about 10 to about 100000 U/kg, preferably from about 10 to 4000 U/kg, more preferably from about 10 to 100 U/kg of lactoperoxidase;
from about 0.01 to about 500 mg/kg, preferably from about 0.1 to about 200 mg/kg of water-soluble iodide salt such as potassium iodide;
and from 0.0005 mg/ml to 200 mg/ml, preferably of from 0.005 mg/ml to 10 mg/ml, most preferably of from 0.05 to 1 mg/ml, of a naturally occurring amino acid selected from the group consisting of: serine, threonine, asparagine, glutamine, isoleucine, proline, or any combination thereof.

In embodiments wherein the composition described herein is a (cutaneous) topical composition, said composition may serve a therapeutical and/or non-therapeutical purpose.

The composition may be a therapeutic topical composition. As used herein, terms such as “therapeutic”, “therapy” and the like, refer to treatments wherein the aim is to change a subjects body or a part of a subjects body from an undesired physiological state, disease or disorder which is caused by an infectious agent, to a desired state, such as a less severe state (e.g., amelioration or palliation), or even back to its normal, healthy state (e.g., restoring the health, the physical integrity and the physical well-being of a subject), to keep it (i.e., not worsening) at said undesired physiological status (e.g., stabilization), or slow down progression to a more severe or worse state compared to said undesired physiological change or disorder. The subject to which the therapeutic topical composition is applied to may have been diagnosed to have a disease or condition or may be suspected of being afflicted by a certain disease or condition. Measurable lessening includes any statistically significant decline in a measurable marker or symptom. Statistically significant as used herein refers to p values below 0.05, which is a commonly accepted cutoff score in statistical analysis as a skilled person appreciates. “Treatment” encompasses both curative treatments and treatments directed to reduce symptoms and/or slow progression and/or stabilize a disease. The compositions disclosed herein have the advantage that the antimicrobial activity is increased and as such the compositions disclosed herein will increase the speed of healing by modulating the microbial load and indirectly the inflammatory state that is due to a microbial nature, with as a result that the ointment, gel, dressing or the like needs to be less frequently replaced, which reduces the discomfort of the patient being treated. Accordingly, the present invention provides for pharmaceutical compositions which are suitable to reduce discomfort, mortality and morbidity.

In addition to favourably modulating any inflammation that may occur due to a decreased bacterial load, the compositions described herein additionally modulate inflammation in a more direct manner. Indeed, the inventors have found that said compositions are capable of decreasing the secretion of pro-inflammatory molecules by immune cells. Yet a further advantage of the compositions described herein was found to reside in the capacity to have a direct neutralizing effect on already secreted pro-inflammatory molecules. In certain preferred embodiments, the compositions described herein decrease the secretion of one or more pro-inflammatory molecules selected from the group consisting of: IL-6 (interleukin 6), tumour necrosis factor alpha (TNFa), and matrix metallopeptidase 9 (MMP9) when compared to the secretion of said one or more pro-inflammatory molecules in absence of the enhancing agents subject of the present disclosure. In complementary preferred embodiments, the compositions described herein neutralise one or more secreted pro-inflammatory molecules selected from the group consisting of: IL-6, TNFa, and MMP9, preferably IL-6 and/or MMP9 when compared to the secretion of said one or more pro-inflammatory molecules in absence of the enhancing agents subject of the present disclosure.

A skilled person is aware that in order to achieve an effective therapeutic treatment, a therapeutically effective dose needs to be administered to said subject. Therefore, in the context of the present disclosure “an effective amount” refers to an amount necessary to obtain a physiological effect. The physiological effect may be achieved by a single dose or by multiple doses. A “therapeutically effective amount” or “therapeutically effective dose” indicates an amount of the composition described herein that when administered brings about a clinical positive response with respect to treatment of a subject afflicted by one or more wounds.

“Diagnosed with”, “diagnosing”, and “diagnosis” are indicative for a process of recognising, deciding on, or concluding on a disease, condition, or (adverse side effect) in a subject on the basis of symptoms and signs and/or from results of various diagnostic procedures (such as, for example, from knowing the presence, absence and/or quantity of one or more biomarkers of or clinical symptoms characteristic for the diagnosed disease or condition). “Diagnosis of” the diseases, conditions, or (adverse) side effects as taught herein in a subject may particularly mean that the subject has such disease or condition. A subject may be diagnosed as not having such despite displaying one or more conventional symptoms or signs reminiscent of such. “Diagnosis of” the diseases or conditions as taught herein in a subject may particularly mean that the subject has a skin disorder. Alternatively, a subject may be diagnosed as not having an particular disease despite displaying one or more conventional symptoms or signs reminiscent of such.

The composition may be a prophylactic topical composition. “Prophylactic compositions” in the present context indicate compositions that are applied to a subject in order to inhibit an impeding disease or condition which at the time of application are not yet characterised by any clinical manifestation. A skilled person appreciates that the goal of the treatment is to prevent occurrence, development and progression of e.g. bacterial infections, such as e.g. in a wound of a subject, i.e., on the skin of a subject. For example, a prophylactic topical composition in the context of the invention may be applied before a burn wound becomes apparent, or as part of a preparative procedure prior to surgery, or as preventive treatment to prevent bedsores. The term “to prevent”, or its alternative forms such as “preventing” or “prevention”, commonly means to keep something from occurring, happening or existing, or to delay the occurrence or onset of something, especially by one or more precautionary (preventative) measures.

The composition may be a cosmetic topical composition. Terms such as “cosmetic”, “cosmetic composition”, and “cosmetic aid” refer to means suitable for increasing the appearance of cleanliness, (personal) hygiene, and/or physical cleanliness. Cosmetic uses or methods as envisaged herein address normal, natural, or physiological processes, and can be distinguished from therapy including curative and prophylactic treatments, the purpose of which is to restore a subject from a pathological state to its original healthy condition, or to at least alleviate the symptoms of pain and suffering caused by the pathology, or to prevent pathology in the first place. Cosmetic uses or methods as intended herein can thus be denoted as “non-therapeutic”. Cosmetic uses or methods as intended herein generally employ cosmetic compositions configured for topical application to the skin. The cosmetic purpose of the composition is not particularly limited and may therefore include cleansing of the skin, preservation of the skin's moisture balance, stimulation of skin metabolism, protection of the skin from harmful environmental factors such as but not limited to UV radiation, and any combination thereof.

The release profile of the contents (i.e. ingredients) of the topical composition is not particularly limited for the present invention, and may be adjusted and optimized by a skilled person who is capable of doing so. Optionally, the topical composition is an immediate release composition. In such embodiments, the ingredients of the composition are released onto the skin of the subject in a non-limiting manner. Optionally, at least about 75% of the peroxidase, peroxide generating system, and enhancing agent are released from the topical composition within about 6 hours of application, preferably within about 4 hours of application, more preferably within about 2 hours of application, more preferably within 1 hour of application, more preferably within 30 minutes of application, most preferably within about 15 minutes of application. Alternatively, the composition is a sustained or controlled release composition. In such embodiments, the ingredients of the composition are released onto the skin of the subject in a limiting manner. Optionally, the composition releases less than 50% of the peroxidase, peroxide generating system, and enhancing agent within 2 hours, preferably less than 50% of the peroxidase, peroxide generating system, and enhancing agent within 4 hours, preferably less than 50% of the peroxidase, peroxide generating system, and enhancing agent within 6 hours, preferably less than 50% of the peroxidase, peroxide generating system, and enhancing agent within 12 hours.

In a further aspect, the composition is envisaged for use as a medicament (both in a therapeutic and prophylactic content). Alternatively worded, the use of the composition described herein is envisaged for the manufacture of a medicament. Yet alternatively worded, use of the composition described herein is envisaged as a medicament.

Optionally, the composition is envisaged for use in treatment of a skin disorder (both in a therapeutic and prophylactic content). Alternatively worded, the use of the composition described herein is envisaged for the manufacture of a medicament for treating a skin disorder. Yet alternatively worded, use of the composition described herein is envisaged as a medicament for treating a skin disorder. The term “skin disorder” as used herein generally refers to any aberrant condition of the skin, including but not limited to inflammatory conditions caused by a microbial infection. A skilled person appreciates that inflammation is a mechanism of disease caused by an infection (i.e. “a microbial infection”). An inflammatory skin disorder caused by a microbial infection originates from an innate immune response to an infection due to a microbe such as, for example, a virus, bacterium, fungus, parasite, or any combination thereof.

By means of illustration and not limitation, viral infections of the skin include shingles (herpes zoster), chickenpox, molluscum contagiosum, warts, measles, and hand, foot, and mouth disease.

By means of illustration and not limitation, bacterial infections of the skin include carbuncles, ecthyma, erythrasma, folliculitis, furuncles, impetigo, lymphadenitis, small skin abscesses (pus-filled pockets in the skin), cellulitis, erysipelas, large skin abscesses, lymphangitis, and necrotizing skin infections.

By means of illustration and not limitation, fungal infections of the skin include body ringworm infections (tinea corporis), tinea pedis (athlete's foot), jock itch, scalp ringworm infections (tinea capitis), tinea versicolor (Pityriasis versicolor), cutaneous candidiasis, and onychomycosis (tinea unguium).

Optionally, the composition is envisaged for use in treatment of a skin wound. Alternatively worded, the use of the composition described herein is envisaged for the manufacture of a medicament for treating a skin wound. Yet alternatively worded, use of the composition described herein is envisaged as a medicament for treating a skin wound. The nature and/or cause of the skin wound is not particularly limiting for the present invention, and includes without limitation acute wounds caused by injury and surgically-induced wounds. The cause of the injury is not limiting for the invention and therefore encompasses both accidental injuries and injuries caused by malintent (i.e., combat wounds).

Optionally, the composition described herein is envisaged for use in treatment of burn wounds. “Burn wound” as used herein refers to a particular kind of tissue injury caused by contact with heat, flame, chemicals, electricity, or radiation. Generally, first degree burns are characterised by redness; second degree burns are characterised by the presence of one or more blistered spots (i.e., vesication); third degree burns are characterised by the presence of necrosis. Throughout the art, burns of the first and second degree are commonly referred to in the art as partial-thickness burns (i.e. destruction of tissue through the epidermis extending to but not through the dermis). Burns of the third degree are commonly referred to as full-thickness burns (i.e. destruction by full extension through the dermis).

Optionally, the pharmaceutical compositions and hydrogels described herein are for use in treatment of chronic wounds. The term “chronic wound” indicates any wound that is not showing adequate signs of healing upon application of a standard wound healing process (e.g., but not limited to, diabetic foot ulcer, venous leg ulcer, pressure ulcer). Thus, wounds can be clinically categorized as acute or chronic based on their time frame of healing. The term “chronic wound” may interchangeably be used with synonymous terms such as but not limited to “hard-to-heal wound”, “difficult-to-heal wound”, “non-healing wound”, and “complex wound”. Chronic wounds may be characterised by a dysregulated healing process by a plethora of factors that prolong one or more wound healing phases.

Accordingly, the invention also envisages use of the composition described herein in method of treatments, such as methods of treating skin disorders, methods of treating wounds such as skin wounds, and methods of treating microbial infections. The methods of treatment comprise administering to a subject the composition described herein. In embodiments wherein the composition is a topical cutaneous composition, the method comprises administering the composition on (a portion of) the skin of a subject. In further embodiments, the method of treatment involves a one time application of the composition to (a portion of) the skin of a subject. In alternative embodiments, the method of treatment involves a monthly, preferably biweekly, more preferably weekly, most preferably daily administration of the composition to (a portion of) the skin of the subject.

The composition subject of the invention may be used in combination with further pharmaceutically active ingredients and/or medicaments. The term “pharmaceutically active ingredient”, interchangeably used throughout the present disclosure with “pharmaceutically active agent” is to be interpreted according to the definition of the term by the World Health organisation: “a substance used in a finished pharmaceutical product (FPP), intended to display pharmacological activity or to otherwise have direct effect in the diagnosis, cure, mitigation, treatment or prevention of disease, or to have direct effect in restoring, correcting or modifying physiological functions in human beings”.

For example, the composition may be used in combination with an analgesic and/or an anti-inflammatory agent. The further pharmaceutically active ingredient may be included in the composition subject of the invention. Alternatively, the further pharmaceutically active ingredient may be comprised in a separate composition, i.e. an additional and physically distinguishable composition. “Analgesic” as used herein is to be interpreted in its broadest interpretation and may therefore refer to any compound, substance, or composition that is able to reduce pain or suppress pain in a subject, and hence any product that is able to achieve analgesia in a subject. Throughout the art, any analgesic may interchangeably be referred to as “painkiller” or “pain reliever”. The analgesic may be used in the treatment of pain and/or in pain prevention (i.e. pain prophylaxis). An analgesic may act on the peripheral and/or central nervous system. In the art, analgesics are commonly classified according to the mechanism of action. Different classes of analgesics include but are not limited to acetaminophen (i.e. paracetamol), nonsteroidal anti-inflammatory drugs (NSAIDs), opioids, muscle-relaxants, anti-anxiety agents, antidepressants, anticonvulsants, and corticosteroids.

It is evident that additional pharmaceutically active agents having an anti-microbial may be further incorporated in the composition. Optionally, the composition subject of the invention may be combined with at least one further antiviral drug, at least one further antibacterial drug, at least one further antifungal drug, or at least one antiparasitic drug.

In a further aspect, the present invention is directed to the use of an enhancing agent as described herein for improving the Minimum Inhibitory Concentration (MIC) and/or the Minimum Bactericidal concentration (MBC) and/or absolute counts and/or enzymatic activity of a pharmaceutical composition comprising a peroxide generating system, a peroxidase, and a halide or pseudohalide, wherein the enhancing agent is as described herein above. In particular embodiments, the enhancing agent is a hydrophilic amino acid, amino acid mimic, or amino acid derivative, characterized by a free amino functional group (—NH₃⁺), free carboxylic acid functional group (—CO₂H), and a side chain comprising a
(i) —OH group;
(ii) —(CH₂)ₙOH group wherein n is 1, 2, or 3;
(iii) —(CH₂)ₙCONH₂ group wherein n is 1, 2, or 3;
(iv) -a cyclic C₄H₉N group; or
(v) a branched alkylgroup.

Also envisaged is the use of an enhancing agent as described herein for improving the Minimum Inhibitory Concentration (MIC) and/or the Minimum Bactericidal concentration (MBC) and/or absolute counts and/or enzymatic of a pharmaceutical composition comprising glucose oxidase, lactoperoxidase, and a water-soluble iodide salt. In particular embodiments, the enhancing agent is a naturally or non-naturally occurring amino acid, preferably the enhancing agent is a naturally occurring amino acid selected from the group consisting of: serine, threonine, asparagine, glutamine, isoleucine, proline, or any combination thereof.

The use for improving the Minimum Inhibitory Concentration (MIC) and/or the Minimum Bactericidal concentration (MBC) and/or absolute counts and/or enzymatic activity of a pharmaceutical composition is an in vitro use. In certain embodiments, the use is an in vitro use for the preservation of skin grafts, cell lines, biomarkers, and/or any other biological sample. The term “in vitro” generally denotes outside, or external to, animal or human body.

The term “ex vivo” typically refers to tissues or cells removed from an animal or human body and maintained or propagated outside the body, e.g., in a culture vessel. The term “in vitro” as used herein should be understood to include “ex vivo”. The term “in vivo” generally denotes inside, on, or internal to, animal or human body.

As used herein, the terms “biological sample” or “sample” refers to a biological material that is isolated from its natural environment. The sample may correspond to or comprise a tissue sample, a biological fluid sample (e.g., blood, plasma), or a cell sample, e.g., a hematopoietic cell sample. The term “biomarker”, often indicated in the art by the term “marker”, is widespread in the art and commonly broadly denotes a biological component or a biological molecule, more particularly an endogenous biological component or molecule, or a detectable portion thereof, whose qualitative and/or quantitative evaluation in a tested subject, such as by means of evaluating a biological sample from the subject, is predictive (e.g. predictive, diagnostic and/or prognostic) or informative with respect to one or more aspects of the tested subjects' phenotype and/or genotype for example with respect to the status of the subject as to a given disease or condition. In certain embodiments wherein the use is an in vitro use for preservation of a biological sample intended to be transferred in a separate method not related to the present invention onto or into a living subject (such as but not limited to a skin graft sample), the composition is may be directly applied to said sample or the sample may be placed in a liquid or semi-liquid environment wherein said environment comprises the composition subject of the invention.

In yet a further aspect, the invention is directed to the use of an enhancing agent as described herein to preserve sterility of a pharmaceutical, prophylactical, or cosmetic composition. In particular embodiments, the enhancing agent is a hydrophilic amino acid, amino acid mimic, or amino acid derivative, characterized by a free amino functional group (—NH₃+), free carboxylic acid functional group (—CO₂H), and a side chain comprising a
(i) —OH group;
(ii) —(CH₂)ₙOH group wherein n is 1, 2, or 3;
(iii) —(CH₂)ₙCONH₂ group wherein n is 1, 2, or 3;
(iv) -a cyclic C₄H₉N group; or
(v) a branched alkylgroup.

Optionally, the use of an enhancing agent as described herein is envisaged for the use of preserving sterility of a pharmaceutical, prophylactical, or cosmetic composition. In particular embodiments, the enhancing agent is a naturally or non-naturally occurring amino acid, preferably the enhancing agent is a naturally occurring amino acid selected from the group consisting of: serine, threonine, asparagine, glutamine, isoleucine, proline, or any combination thereof. “Sterility” as used herein refers to the complete, or substantially complete, absence of any living organism, such as a microbial organism in a liquid or on a surface.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims. The herein disclosed aspects and embodiments of the invention are further supported by the following non-limiting examples. The following specific experimental examples are provided in support of the claimed invention but are not to be seen as limiting the scope of the invention.

### EXAMPLES

### Example 1. Enzymatic Activity Assays

### 1.1. Glucose Oxidase (GOX) Activity

The natural enzyme GOX is an oxidoreductase that catalyses the oxidation of glucose to gluconic acid and hydrogen peroxide (H₂O₂) in the presence of water and air. To measure the enzymatic activity of GOX, a standard spectrophotometric assay is performed. In brief, a second oxidoreductase enzyme, peroxidase, and a chromogenic oxygen acceptor, o-dianisidine, are added to the former reaction mix (GOX+glucose+water+air) so that the formation of a coloured compound is achieved that can in turn be measured by spectrophotometry. GOX was tested in 4 different concentrations, i.e., 100, 50, 10 and 1 mg/ml. Each reaction mixture was tested in duplo. In the event GOX activity was evaluated in the presence of a potentiator candidate (=a molecule that might be able to augment the enzymatic activity of GOX), the latter was added in a low and high concentration ranging from 100 to 0.1 mg/ml. The blank consisted of the reaction mix in which the enzymes were absent. After mixing all reaction elements, absorbance was immediately measured with a plate reader (Spectramax ID3, Molecular Devices) every minute for 5 minutes at 35° C. For each combination evaluated, this test was repeated at least 2 times. Results were further analysed in MS Excel and expressed as U/g. Mean blank-corrected data±SD were plotted. Statistical significance compared to the combination without potentiator candidate (reference) was calculated using students' t-tests. Significance is achieved when the obtained p-value is lower than 0,05. Significance levels were defined as p<0.05=*; p<0.01=** and p<0.001=***.

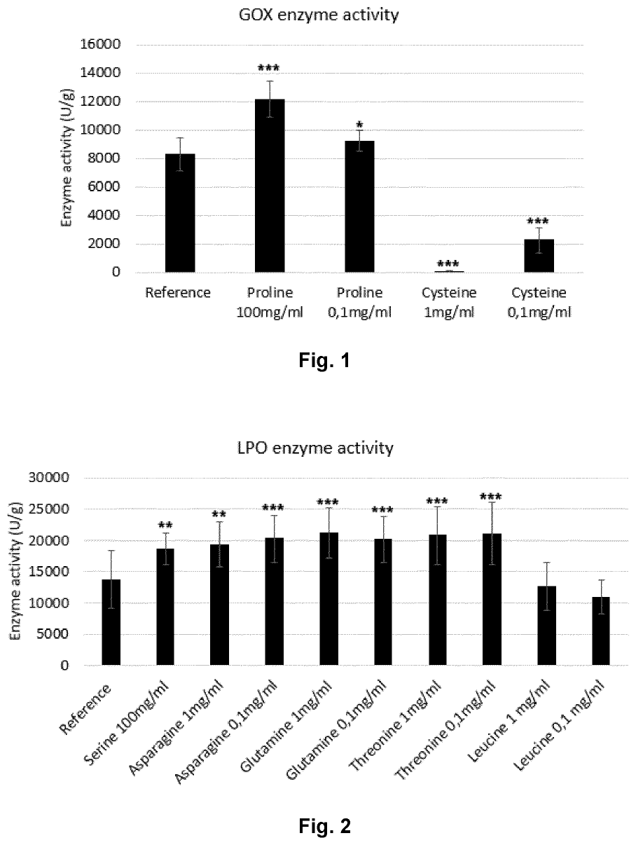

Results indicated that the addition of proline (0.1-100 mg/ml) to the GOX enzyme reaction solution significantly increased the enzymatic activity of GOX when compared to that of the reference (no potentiator candidate) (FIG. 1):
Proline (100 mg/ml): resulted in a significant increase of GOX activity compared to that of the reference. A significance of p<0.001 was obtained.
Proline (0.1 mg/ml): resulted in a significant increase of GOX activity compared to that of the reference. A significance of p<0.05 was obtained.

In contrast, the addition of other amino acids with a similar structure to proline—such as cysteine—did result in a significant lower GOX activity when compared to that of the reference (FIG. 1). Therefore, the increase in GOX activity described in the above is not considered group specific (amino acids in general/proline-like amino acids).

### 1.2. Lactoperoxidase (LPO) Activity

Lactoperoxidase is a member of the heme peroxidase family of enzymes that, together with a catalytic anion (such as I⁻, derived from KI, present in the substrate), will catalyse the oxidation of H₂O₂, leading to hypojodite (IO⁻). This hypojodite is a reactive oxygen species (ROS). To measure the enzymatic activity of LPO, a standard spectrophotometric assay is performed. In brief, H₂O₂ combined with 2,2′-azino-bis (3-ethylbenzothiazoline-6-sulfonzuur (ABTS) is catalysed by LPO so that the formation of a coloured compound is achieved that can in turn be measured by spectrophotometry. LPO was tested in 3 different concentrations, i.e., 50, 10 and 1 mg/ml. Each reaction mixture was tested in duplo. In the event LPO activity was evaluated in the presence of a potentiator candidate (=a molecule that might be able to augment the enzymatic activity of LPO), the latter was added in a low and high concentration ranging from 100 to 0.1 mg/ml. The blank consisted of the reaction mix in which H₂O₂ was absent. The absorbance was immediately measured with a plate reader (Spectramax ID3, Molecular Devices) every 12 seconds for 5 minutes at 20° C. For each combination evaluated, this test was repeated at least 2 times. Results were further analysed in MS Excel and expressed as U/g. Mean blank-corrected data±SD were plotted. Statistical significance compared to the combination without potentiator candidate (reference) was calculated using students' t-tests. Significance is achieved when the obtained p-value is lower than 0,05. Significance levels were defined as p<0.05=*; p<0.01=** and p<0.001=***.

Results indicated that the addition of serine (100 mg/ml), asparagine (1-0.1 mg/ml), glutamine (0.1 mg/ml) and threonine (1-0.1 mg/ml) to the LPO enzyme reaction solution significantly increased the enzymatic activity of LPO when compared to that of the reference (no potentiator candidate) (FIG. 2):
Serine (100 mg/ml) and asparagine (1 mg/ml): resulted in a significant increase of LPO activity compared to that of the reference. A significance of p<0.01 was obtained.
Asparagine (0.1 mg/ml), glutamine (0.1 and 1 mg/ml) and threonine (0.1 and 1 mg/ml): resulted in a significant increase of LPO activity compared to that of the reference. A significance of p<0.001 was obtained.

In contrast, the addition of other amino acids—such as leucine—did not result in a significant difference in LPO activity when compared to that of the reference (FIG. 2). Therefore, the increase in LPO activity described above is not considered group specific.

### Example 2. Antimicrobial Activity Assays

### 2.1. MIC Determination

The minimum inhibitory concentration (MIC) is defined as the lowest concentration of an antimicrobial agent that results in a complete inhibition of microbial growth (determined via visual evaluation or absorbance measurements) after overnight incubation. This endpoint measurement is used to define the microbial susceptibility towards an antimicrobial agent. Here, the antimicrobial agent is a combination of the natural enzymes GOX+LPO. The MIC of this enzyme combination GOX+LPO was studied as such and when combined with a potentiator candidate (=a molecule that does not possess an antimicrobial effect as such, but is able to augment the antimicrobial effect of an antimicrobial agent).

To determine the MIC of the described combinations, a classical broth microdilution assay was used to determine the susceptibility of a series of microbes commonly detected in infected wounds, including (a) the Gram-positive bacterium Staphylococcus aureus ATCC® 6538™ (results shown below), (b) the Gram-negative bacterium Pseudomonas aeruginosa ATCC®9027™ (results shown below), (c) the yeast Candida albicans ATCC®10231™ (results shown below), and (d) the fungus Aspergillus niger ATCC®16404™ (results shown below). In brief, a 1% serial dilution of the enzymatic GOX+LPO solution was prepared in a transparent, flat-bottomed 96-well test plate with final concentrations ranging from 0.005 to 0.00000977% (v/v). To each well, a fixed volume of the enzymatic substrate containing 3,3% (w/v) glucose and 0,084% (w/v) potassium iodide, as well as the microbial inoculum with an OD₆₀₀ of 0.1 was added. In the event the enzyme combination was evaluated in the presence of a potentiator candidate, the latter was added together with the enzymatic substrate in a final concentration ranging from 100 to 0,00125% (v/v). For each combination evaluated, a blank (no addition of microbial inoculum) was included. Positive (inoculum only) and negative (background solution only) were included to evaluate the performance of the test and to select the enzymatic GOX+LPO concentration at which no microbial growth is detected (i.e., absorbance equal to that of the background solution only). Then, the 96-well test plate was incubated for 24 hours at 37° C. (S. aureus, P. aeruginosa and C. albicans) or 25° C. (A. niger). After incubation, the absorbance of each well was measured using a plate spectrophotometer (Spectramax ID3, Molecular Devices). For each combination evaluated, this test was repeated at least 5 times. The collected data were further analyzed in MS Excel. Mean absorbance data were visualized in a table format in which the MIC was highlighted.

2.1.1. S. aureus

Results indicated that addition of serine and isoleucine in a final concentration ranging from 0.0025% to 0.00125% (v/v) to the enzymatic GOX+LPO combination resulted in a difference in MIC when compared to that of the reference (no potentiator candidate), illustrated a difference in S. aureus susceptibility (Table 1):
Serine (0.0025% to 0.00125%): resulted in a 2× increase in MIC when compared to that of the reference or the solution where guaiacol was added as enhancing agent, meaning that only 0.0000391% (v/v) instead of 0.0000781% (v/v) GOX+LPO enzyme solution is needed to completely inhibit the growth of S. aureus. In other words, S. aureus showed to be more susceptible when serine (0.0025% to 0.00125%) was added to the reaction mix containing 0.005%-0.0000391% (v/v) GOX+LPO enzyme solution.
Isoleucine (0.0025% to 0.00125%): resulted in a 2× increase in MIC when compared to that of the reference or the solution where guaiacol was added as enhancing agent, meaning that only 0.0000391% (v/v) instead of 0.0000781% (v/v) GOX+LPO enzyme solution is needed to completely inhibit the growth of S. aureus. In other words, S. aureus showed to be more susceptible when isoleucine (0.0025% to 0.00125%) was added to the reaction mix containing 0.005%-0.0000391% (v/v) GOX+LPO enzyme solution.

When the concentrations of the enhancing molecules were increased, the next results are obtained (Table 2):
Proline (0.01%): resulted in a 4× increase in MIC when compared to that of the reference or the solution where guaiacol was added as enhancing agent, meaning that only 0.0000195% (v/v) instead of 0.0000781% (v/v) GOX+LPO enzyme solution is needed to completely inhibit the growth of S. aureus. In other words, S. aureus showed to be more susceptible when proline (0.01%) was added to the reaction mix containing 0.005%-0.0000195% (v/v) GOX+LPO enzyme solution.
Proline (10%): resulted in a 2× increase in MIC when compared to that of the reference or the solution where guaiacol was added as enhancing agent, meaning that only 0.0000391% (v/v) instead of 0.0000781% (v/v) GOX+LPO enzyme solution is needed to completely inhibit the growth of S. aureus. In other words, S. aureus showed to be more susceptible when proline (10%) was added to the reaction mix containing 0.005%-0.0000391% (v/v) GOX+LPO enzyme solution.
Serine (10%): resulted in a growth inhibition in all tested GOX+LPO enzyme solution concentrations ranging from 0.005 to 0.0000977% (v/v). As such, the MIC is equal or lower than 0.0000977% (v/v) (no smaller concentrations tested) which in turn indicates a 6× difference at least. In other words, S. aureus showed to be more susceptible when serine (10%) was added to the reaction mix containing 0.005%-0.00000977% (v/v) GOX+LPO enzyme solution.
Serine (0.01% to 0.005%): resulted in a 2× increase in MIC when compared to that of the reference or the solution where guaiacol was added as enhancing agent, meaning that only 0.0000391% (v/v) instead of 0.0000781% (v/v) GOX+LPO enzyme solution is needed to completely inhibit the growth of S. aureus. In other words, S. aureus showed to be more susceptible when serine (0.01%-0.005%) was added to the reaction mix containing 0.005%-0.0000391% (v/v) GOX+LPO enzyme solution.
Threonine (2%): resulted in a 4× increase in MIC when compared to that of the reference or the solution where guaiacol was added as enhancing agent, meaning that only 0.0000195% (v/v) instead of 0.0000781% (v/v) GOX+LPO enzyme solution is needed to completely inhibit the growth of S. aureus. In other words, S. aureus showed to be more susceptible when threonine (2%) was added to the reaction mix containing 0.005%-0.0000195% (v/v) GOX+LPO enzyme solution.
Threonine (0.01%): resulted in a 2× increase in MIC when compared to that of the reference or the solution where guaiacol was added as enhancing agent, meaning that only 0.0000391% (v/v) instead of 0.0000781% (v/v) GOX+LPO enzyme solution is needed to completely inhibit the growth of S. aureus. In other words, S. aureus showed to be more susceptible when threonine (0.01%) was added to the reaction mix containing 0.005%-0.0000391% (v/v) GOX+LPO enzyme solution.
Glutamine (1% to 0.01%): resulted in a 2× increase in MIC when compared to that of the reference or the solution where guaiacol was added as enhancing agent, meaning that only 0.0000391% (v/v) instead of 0.0000781% (v/v) GOX+LPO enzyme solution is needed to completely inhibit the growth of S. aureus. In other words, S. aureus showed to be more susceptible when glutamine (1%-0.01%) was added to the reaction mix containing 0.005%-0.0000391% (v/v) GOX+LPO enzyme solution.
Asparagine (0.5% to 0.01%): resulted in a 2× increase in MIC when compared to that of the reference or the solution where guaiacol was added as enhancing agent, meaning that only 0.0000391% (v/v) instead of 0.0000781% (v/v) GOX+LPO enzyme solution is needed to completely inhibit the growth of S. aureus. In other words, S. aureus showed to be more susceptible when asparagine (0.5%-0.01%) was added to the reaction mix containing 0.005%-0.0000391% (v/v) GOX+LPO enzyme solution.
Isoleucine (0.5% to 0.005%): resulted in a 2× increase in MIC when compared to that of the reference or the solution where guaiacol was added as enhancing agent, meaning that only 0.0000391% (v/v) instead of 0.0000781% (v/v) GOX+LPO enzyme solution is needed to completely inhibit the growth of S. aureus. In other words, S. aureus showed to be more susceptible when isoleucine (0.5%-0.005%) was added to the reaction mix containing 0.005%-0.0000391% (v/v) GOX+LPO enzyme solution.
   2.1.2. P. aeruginosa

Results indicated that addition of proline and serine in a final concentration ranging from 0.0025% to 0.00125% (v/v) to the enzymatic GOX+LPO combination resulted in a difference in MIC when compared to that of the reference (no potentiator candidate), illustrating a difference in P. aeruginosa susceptibility (Table 3):
Serine (0.0025-0.00125%): resulted in a 2× increase in MIC when compared to that of the reference or the solution where guaiacol was added as enhancing agent, meaning that only 0.000625% (v/v) instead of 0.00125% (v/v) GOX+LPO enzyme solution is needed to completely inhibit the growth of P. aeruginosa. In other words, P. aeruginosa showed to be more susceptible when serine (0.0025-0.00125%) was added to the reaction mix containing 0.005%-0.000625% (v/v) GOX+LPO enzyme solution.
Proline (0.0025-0.00125%): resulted in a 2× increase in MIC when compared to that of the reference or the solution where guaiacol was added as enhancing agent, meaning that only 0.000625% (v/v) instead of 0.00125% (v/v) GOX+LPO enzyme solution is needed to completely inhibit the growth of P. aeruginosa. In other words, P. aeruginosa showed to be more susceptible when proline (0,0025-0.00125%) was added to the reaction mix containing 0.005%-0.000625% (v/v) GOX+LPO enzyme solution.

When the concentrations of the enhancing molecules were increased, the next results are obtained (Table 4):
Serine (0.01-0.005%): resulted in a 2× increase in MIC when compared to that of the reference or the solution where guaiacol was added as enhancing agent, meaning that only 0.000625% (v/v) instead of 0.00125% (v/v) GOX+LPO enzyme solution is needed to completely inhibit the growth of P. aeruginosa. In other words, P. aeruginosa showed to be more susceptible when serine (0.01-0.005%) was added to the reaction mix containing 0.005%-0.000625% (v/v) GOX+LPO enzyme solution.
Proline (0.01-0.005%): resulted in a 2× increase in MIC when compared to that of the reference or the solution where guaiacol was added as enhancing agent, meaning that only 0.000625% (v/v) instead of 0.00125% (v/v) GOX+LPO enzyme solution is needed to completely inhibit the growth of P. aeruginosa. In other words, P. aeruginosa showed to be more susceptible when proline (0.01-0.005%) was added to the reaction mix containing 0.005%-0.000625% (v/v) GOX+LPO enzyme solution.

Results indicated that addition of threonine in a final concentration ranging from 1% to 0.10% (v/v) to the enzymatic GOX+LPO combination resulted in a difference in MIC when compared to that of the reference (no potentiator candidate), illustrating a difference in P. aeruginosa susceptibility (Table 5):
Threonine (1%): resulted in a 8× increase in MIC when compared to that of the reference, meaning that only 0.000156% (v/v) instead of 0.00125% (v/v) GOX+LPO enzyme solution is needed to completely inhibit the growth of P. aeruginosa. In other words, P. aeruginosa showed to be more susceptible when threonine (1%) was added to the reaction mix containing 0,005%-0,000156% (v/v) GOX+LPO enzyme solution.
Threonine (0,1%): resulted in a 2× increase in MIC when compared to that of the reference, meaning that only 0.000625% (v/v) instead of 0.00125% (v/v) GOX+LPO enzyme solution is needed to completely inhibit the growth of P. aeruginosa. In other words, P. aeruginosa showed to be more susceptible when threonine (0,1%) was added to the reaction mix containing 0,005%-0,000625% (v/v) GOX+LPO enzyme solution.

Results indicated that addition of isoleucine in a final concentration ranging from 1% to 0.1% (v/v) to the enzymatic GOX+LPO combination resulted in a difference in MIC when compared to that of the reference (no potentiator candidate), illustrating a difference in P. aeruginosa susceptibility (Table 6):
Isoleucine (1%): resulted in a 8× increase in MIC when compared to that of the reference, meaning that only 0.000156% (v/v) instead of 0.00125% (v/v) GOX+LPO enzyme solution is needed to completely inhibit the growth of P. aeruginosa. In other words, P. aeruginosa showed to be more susceptible when isoleucine (1%) was added to the reaction mix containing 0,005%-0,000156% (v/v) GOX+LPO enzyme solution.
Isoleucine (0,1%): resulted in a 2× increase in MIC when compared to that of the reference, meaning that only 0.000625% (v/v) instead of 0.00125% (v/v) GOX+LPO enzyme solution is needed to completely inhibit the growth of P. aeruginosa. In other words, P. aeruginosa showed to be more susceptible when isoleucine (0,1%) was added to the reaction mix containing 0,005%-0,000625% (v/v) GOX+LPO enzyme solution.

Results indicated that addition of glutamine in a final concentration ranging from 1% to 0.1% (v/v) to the enzymatic GOX+LPO combination resulted in a difference in MIC when compared to that of the reference (no potentiator candidate), illustrating a difference in P. aeruginosa susceptibility (Table 7):
Glutamine (1%): resulted in a 8× increase in MIC when compared to that of the reference, meaning that only 0.000156% (v/v) instead of 0.00125% (v/v) GOX+LPO enzyme solution is needed to completely inhibit the growth of P. aeruginosa. In other words, P. aeruginosa showed to be more susceptible when glutamine (1%) was added to the reaction mix containing 0,005%-0,000156% (v/v) GOX+LPO enzyme solution.
Glutamine (0,1%): resulted in a 2× increase in MIC when compared to that of the reference, meaning that only 0.000625% (v/v) instead of 0.00125% (v/v) GOX+LPO enzyme solution is needed to completely inhibit the growth of P. aeruginosa. In other words, P. aeruginosa showed to be more susceptible when glutamine (0,1%) was added to the reaction mix containing 0,005%-0,000625% (v/v) GOX+LPO enzyme solution.

Results indicated that addition of asparagine in a final concentration ranging from 1% to 0.1% (v/v) to the enzymatic GOX+LPO combination resulted in a difference in MIC when compared to that of the reference (no potentiator candidate), illustrating a difference in P. aeruginosa susceptibility (Table 8):
Asparagine (1%): resulted in a 8× increase in MIC when compared to that of the reference, meaning that only 0.000156% (v/v) instead of 0.00125% (v/v) GOX+LPO enzyme solution is needed to completely inhibit the growth of P. aeruginosa. In other words, P. aeruginosa showed to be more susceptible when asparagine (1%) was added to the reaction mix containing 0,005%-0,000156% (v/v) GOX+LPO enzyme solution.
Asparagine (0,1%): resulted in a 2× increase in MIC when compared to that of the reference, meaning that only 0.000625% (v/v) instead of 0.00125% (v/v) GOX+LPO enzyme solution is needed to completely inhibit the growth of P. aeruginosa. In other words, P. aeruginosa showed to be more susceptible when asparagine (0,1%) was added to the reaction mix containing 0,005%-0,000625% (v/v) GOX+LPO enzyme solution.
   2.1.3. C. albicans

Results indicated that addition of isoleucine in a final concentration of 0.01% (v/v) to the enzymatic GOX+LPO combination resulted in a difference in MIC when compared to that of the reference (no potentiator candidate), illustrating a difference in C. albicans susceptibility (Table 9):
Isoleucine (0.01%): resulted in a 4× increase in MIC when compared to that of the reference, and a 2× increase in MIC when compared to the enhancing molecule guaiacol, meaning that only 0.0000391% (v/v) instead of 0.000156% (v/v) GOX+LPO enzyme solution is needed to completely inhibit the growth of C. albicans. In other words, C. albicans showed to be more susceptible when isoleucine (0.01%) was added to the reaction mix containing 0.005%-0.0000391% (v/v) GOX+LPO enzyme solution.
   2.1.4. A. niger

Results indicated that addition of proline in a final concentration ranging from 1% to 0.01% (v/v) to the enzymatic GOX+LPO combination resulted in a difference in MIC when compared to that of the reference (no potentiator candidate), illustrating a difference in A. niger susceptibility (Table 10):
Proline (1%-0.01%): resulted in a 2× increase in MIC when compared to that of the reference, meaning that only 0.000156% (v/v) instead of 0.000313% (v/v) GOX+LPO enzyme solution is needed to completely inhibit the growth of A. niger. In other words, A. niger showed to be more susceptible when proline (1%-0.01%) was added to the reaction mix containing 0.005%-0.000156% (v/v) GOX+LPO enzyme solution.

Results indicated that addition of serine in a final concentration ranging from 1% to 0.01% (v/v) to the enzymatic GOX+LPO combination resulted in a difference in MIC when compared to that of the reference (no potentiator candidate), illustrating a difference in A. niger susceptibility (Table 11):
Serine (1%-0.01%): resulted in a 2× increase in MIC when compared to that of the reference, meaning that only 0.000156% (v/v) instead of 0.000313% (v/v) GOX+LPO enzyme solution is needed to completely inhibit the growth of A. niger. In other words, A. niger showed to be more susceptible when serine (1%-0.01%) was added to the reaction mix containing 0.005%-0.000156% (v/v) GOX+LPO enzyme solution.

Results indicated that addition of threonine in a final concentration ranging from 1% to 0.01% (v/v) to the enzymatic GOX+LPO combination resulted in a difference in MIC when compared to that of the reference (no potentiator candidate), illustrating a difference in A. niger susceptibility (Table 12):
Threonine (1%-0.01%): resulted in a 2× increase in MIC when compared to that of the reference, meaning that only 0.000156% (v/v) instead of 0.000313% (v/v) GOX+LPO enzyme solution is needed to completely inhibit the growth of A. niger. In other words, A. niger showed to be more susceptible when threonine (1%-0.01%) was added to the reaction mix containing 0.005%-0.000156% (v/v) GOX+LPO enzyme solution.

Results indicated that addition of glutamine in a final concentration ranging from 1% to 0.01% (v/v) to the enzymatic GOX+LPO combination resulted in a difference in MIC when compared to that of the reference (no potentiator candidate), illustrating a difference in A. niger susceptibility (Table 13):
Glutamine (1%-0.01%): resulted in a 2× increase in MIC when compared to that of the reference, meaning that only 0.000156% (v/v) instead of 0.000313% (v/v) GOX+LPO enzyme solution is needed to completely inhibit the growth of A. niger. In other words, A. niger showed to be more susceptible when glutamine (1%-0.01%) was added to the reaction mix containing 0.005%-0.000156% (v/v) GOX+LPO enzyme solution.

Results indicated that addition of asparagine in a final concentration ranging from 1% to 0.05% (v/v) to the enzymatic GOX+LPO combination resulted in a difference in MIC when compared to that of the reference (no potentiator candidate), illustrating a difference in A. niger susceptibility (Table 14):
Asparagine (1%-0.05%): resulted in a 2× increase in MIC when compared to that of the reference, meaning that only 0.000156% (v/v) instead of 0.000313% (v/v) GOX+LPO enzyme solution is needed to completely inhibit the growth of A. niger. In other words, A. niger showed to be more susceptible when asparagine (1%-0.05%) was added to the reaction mix containing 0.005%-0.000156% (v/v) GOX+LPO enzyme solution.

Results indicated that addition of isoleucine in a final concentration ranging from 1% to 0.005% (v/v) to the enzymatic GOX+LPO combination resulted in a difference in MIC when compared to that of the reference (no potentiator candidate), illustrating a difference in A. niger susceptibility (Table 15):
Isoleucine (1%-0.005%): resulted in a 2× increase in MIC when compared to that of the reference, meaning that only 0.000156% (v/v) instead of 0.000313% (v/v) GOX+LPO enzyme solution is needed to completely inhibit the growth of A. niger. In other words, A. niger showed to be more susceptible when isoleucine (1%-0.005%) was added to the reaction mix containing 0.005%-0.000156% (v/v) GOX+LPO enzyme solution.

### 2.2. MBC Determination

The minimal bactericidal concentration (MBC) is defined as the lowest concentration of an antimicrobial agent that results in a complete killing of the microbial population (determined via standard CFU enumeration techniques) after overnight incubation. This endpoint measurement is used, together with the MIC (see above), to define the microbial susceptibility towards an antimicrobial agent. Here, the antimicrobial agent is a combination of the natural enzymes GOX+LPO. The MBC of this enzyme combination GOX+LPO was studied as such and when combined with a potentiator candidate (=a molecule that does not possess an antimicrobial effect as such, but is able to augment the antimicrobial effect of an antimicrobial agent).

To determine the MBC of the described combinations, a classical broth microdilution assay combined with standard CFU enumeration techniques was used to determine the susceptibility of a series of microbes commonly detected in infected wounds, including (a) the Gram-positive bacterium Staphylococcus aureus ATCC® 6538™ (results shown below), (b) the Gram-negative bacterium Pseudomonas aeruginosa ATCC®9027™ (results shown below), (c) the yeast Candida albicans ATCC®10231™ (results shown below), and (d) the fungus Aspergillus niger ATCC®16404™ (results shown below). In brief, the broth microdilution assay was set up as previously described (see 2.1). After incubation, the MBC was determined by investigating the presence of viable microorganisms at the MIC and following higher concentrations (MICx2, MICx4, and so on) using standard CFU enumeration techniques. For each combination evaluated, this test was repeated at least 5 times. The collected data were further analyzed in MS Excel. Mean concentration data (expressed as log CFU/ml) were visualized in a table format in which the MBC was highlighted.

2.2.1. S. aureus

Results indicated that addition of serine and isoleucine in a final concentration ranging from 0.0025% to 0.00125% (v/v) to the enzymatic GOX+LPO combination resulted in a difference in MBC when compared to that of the reference (no potentiator candidate), illustrated a difference in S. aureus susceptibility (Table 16):
Serine (0.0025%-0.00125%): resulted in a 4× increase in MBC when compared to that of the reference, and a 2× increase in MBC when compared to the enhancing molecule guaiacol, meaning that only 0.0000391% (v/v) instead of 0.0000781% (v/v) GOX+LPO enzyme solution is needed to completely inhibit the growth of S. aureus. In other words, S. aureus showed to be more susceptible when serine (0.0025%-0.00125%) was added to the reaction mix containing 0.005%-0.0000391% (v/v) GOX+LPO enzyme solution.
Isoleucine (0.00125%): resulted in a 4× increase in MBC when compared to that of the reference, and a 2× increase in MBC when compared to the enhancing molecule guaiacol, meaning that only 0.0000391% (v/v) instead of 0.0000781% (v/v) GOX+LPO enzyme solution is needed to completely inhibit the growth of S. aureus. In other words, S. aureus showed to be more susceptible when isoleucine (0.00125%) was added to the reaction mix containing 0.005%-0.0000391% (v/v) GOX+LPO enzyme solution.

When the concentrations of the enhancing molecules were increased, the next results are obtained (Table 17):
Serine (10%-0.005%): resulted in a 4× increase in MBC when compared to that of the reference, and a 2× increase in MBC when compared to the enhancing molecule guaiacol, meaning that only 0.0000391% (v/v) instead of 0.00007811% (v/v) GOX+LPO enzyme solution is needed to completely inhibit the growth of S. aureus. In other words, S. aureus showed to be more susceptible when serine (10%-0.005%) was added to the reaction mix containing 0.005%-0.0000391% (v/v) GOX+LPO enzyme solution.
Isoleucine (0.005%): resulted in a 4× increase in MBC when compared to that of the reference, and a 2× increase in MBC when compared to the enhancing molecule guaiacol, meaning that only 0.0000391% (v/v) instead of 0.0000781% (v/v) GOX+LPO enzyme solution is needed to completely inhibit the growth of S. aureus. In other words, S. aureus showed to be more susceptible when isoleucine (0.005%) was added to the reaction mix containing 0.005%-0.0000391% (v/v) GOX+LPO enzyme solution.
Glutamine (0.1%): resulted in a 4× increase in MBC when compared to that of the reference, and a 2× increase in MBC when compared to the enhancing molecule guaiacol, meaning that only 0.0000391% (v/v) instead of 0.0000781% (v/v) GOX+LPO enzyme solution is needed to completely inhibit the growth of S. aureus. In other words, S. aureus showed to be more susceptible when glutamine (0.1%) was added to the reaction mix containing 0.005%-0.0000391% (v/v) GOX+LPO enzyme solution.
   2.2.2. P. aeruginosa

Results indicated that addition of proline and serine in a final concentration ranging from 0.0025% to 0.00125% (v/v) to the enzymatic GOX+LPO combination resulted in a difference in MBC when compared to that of the reference (no potentiator candidate), illustrating a difference in P. aeruginosa susceptibility (Table 18):
Serine (0.0025-0.00125%): resulted in a 2× increase in MBC when compared to that of the reference or the solution where guaiacol was added as enhancing agent, meaning that only 0.000625% (v/v) instead of 0.00125% (v/v) GOX+LPO enzyme solution is needed to completely inhibit the growth of P. aeruginosa. In other words, P. aeruginosa showed to be more susceptible when serine (0.0025-0.00125%) was added to the reaction mix containing 0.005%-0.000625% (v/v) GOX+LPO enzyme solution.
Proline (0.0025-0.00125%): resulted in a 2× increase in MBC when compared to that of the reference or the solution where guaiacol was added as enhancing agent, meaning that only 0.000625% (v/v) instead of 0.00125% (v/v) GOX+LPO enzyme solution is needed to completely inhibit the growth of P. aeruginosa. In other words, P. aeruginosa showed to be more susceptible when proline (0.0025-0.00125%) was added to the reaction mix containing 0.005%-0.000625% (v/v) GOX+LPO enzyme solution.

When the concentrations of the enhancing molecules were increased, the next results are obtained (Table 19):
Serine (0.01-0.005%): resulted in a 2× increase in MBC when compared to that of the reference or the solution where guaiacol was added as enhancing agent, meaning that only 0.000625% (v/v) instead of 0.00125% (v/v) GOX+LPO enzyme solution is needed to completely inhibit the growth of P. aeruginosa. In other words, P. aeruginosa showed to be more susceptible when serine (0.01-0.005%) was added to the reaction mix containing 0.005%-0.000625% (v/v) GOX+LPO enzyme solution.
Proline (0.01-0.005%): resulted in a 2× increase in MBC when compared to that of the reference or the solution where guaiacol was added as enhancing agent, meaning that only 0.000625% (v/v) instead of 0.00125% (v/v) GOX+LPO enzyme solution is needed to completely inhibit the growth of P. aeruginosa. In other words, P. aeruginosa showed to be more susceptible when proline (0.01-0.005%) was added to the reaction mix containing 0.005%-0.000625% (v/v) GOX+LPO enzyme solution.

Results indicated that addition of threonine in a final concentration ranging from 1% to 0.1% (v/v) to the enzymatic GOX+LPO combination resulted in a difference in MBC when compared to that of the reference (no potentiator candidate), illustrating a difference in P. aeruginosa susceptibility (Table 20):
Threonine (1%): resulted in a 8× increase in MBC when compared to that of the reference, meaning that only 0.000156% (v/v) instead of 0.00125% (v/v) GOX+LPO enzyme solution is needed to completely inhibit the growth of P. aeruginosa. In other words, P. aeruginosa showed to be more susceptible when threonine (1%) was added to the reaction mix containing 0,005%-0,000156% (v/v) GOX+LPO enzyme solution.
Threonine (0,1%): resulted in a 2× increase in MBC when compared to that of the reference, meaning that only 0.000625% (v/v) instead of 0.00125% (v/v) GOX+LPO enzyme solution is needed to completely inhibit the growth of P. aeruginosa. In other words, P. aeruginosa showed to be more susceptible when threonine (0,1%) was added to the reaction mix containing 0,005%-0,000625% (v/v) GOX+LPO enzyme solution.

Results indicated that addition of isoleucine in a final concentration ranging from 1% to 0.1% (v/v) to the enzymatic GOX+LPO combination resulted in a difference in MBC when compared to that of the reference (no potentiator candidate), illustrating a difference in P. aeruginosa susceptibility (Table 21):
Isoleucine (1%): resulted in a 8× increase in MBC when compared to that of the reference, meaning that only 0.000156% (v/v) instead of 0.00125% (v/v) GOX+LPO enzyme solution is needed to completely inhibit the growth of P. aeruginosa. In other words, P. aeruginosa showed to be more susceptible when isoleucine (1%) was added to the reaction mix containing 0,005%-0,000156% (v/v) GOX+LPO enzyme solution.
Isoleucine (0,1%): resulted in a 2× increase in MBC when compared to that of the reference, meaning that only 0.000625% (v/v) instead of 0.00125% (v/v) GOX+LPO enzyme solution is needed to completely inhibit the growth of P. aeruginosa. In other words, P. aeruginosa showed to be more susceptible when isoleucine (0,1%) was added to the reaction mix containing 0,005%-0,000625% (v/v) GOX+LPO enzyme solution.

Results indicated that addition of glutamine in a final concentration ranging from 1% to 0.1% (v/v) to the enzymatic GOX+LPO combination resulted in a difference in MBC when compared to that of the reference (no potentiator candidate), illustrating a difference in P. aeruginosa susceptibility (Table 22):
Glutamine (1%): resulted in a 8× increase in MBC when compared to that of the reference, meaning that only 0.000156% (v/v) instead of 0.00125% (v/v) GOX+LPO enzyme solution is needed to completely inhibit the growth of P. aeruginosa. In other words, P. aeruginosa showed to be more susceptible when glutamine (1%) was added to the reaction mix containing 0,005%-0,000156% (v/v) GOX+LPO enzyme solution.
Glutamine (0,1%): resulted in a 2× increase in MBC when compared to that of the reference, meaning that only 0.000625% (v/v) instead of 0.00125% (v/v) GOX+LPO enzyme solution is needed to completely inhibit the growth of P. aeruginosa. In other words, P. aeruginosa showed to be more susceptible when glutamine (0,1%) was added to the reaction mix containing 0,005%-0,000625% (v/v) GOX+LPO enzyme solution.

Results indicated that addition of asparagine in a final concentration ranging from 1% to 0.1% (v/v) to the enzymatic GOX+LPO combination resulted in a difference in MBC when compared to that of the reference (no potentiator candidate), illustrating a difference in P. aeruginosa susceptibility (Table 23):
Asparagine (1%): resulted in a 8× increase in MBC when compared to that of the reference, meaning that only 0.000156% (v/v) instead of 0.00125% (v/v) GOX+LPO enzyme solution is needed to completely inhibit the growth of P. aeruginosa. In other words, P. aeruginosa showed to be more susceptible when asparagine (1%) was added to the reaction mix containing 0,005%-0,000156% (v/v) GOX+LPO enzyme solution.
Asparagine (0,1%): resulted in a 2× increase in MBC when compared to that of the reference, meaning that only 0.000625% (v/v) instead of 0.00125% (v/v) GOX+LPO enzyme solution is needed to completely inhibit the growth of P. aeruginosa. In other words, P. aeruginosa showed to be more susceptible when asparagine (0,1%) was added to the reaction mix containing 0,005%-0,000625% (v/v) GOX+LPO enzyme solution.
   2.2.3. C. albicans

Results indicated that addition of serine and isoleucine in a final concentration of 0.01% (v/v) to the enzymatic GOX+LPO combination resulted in a difference in MBC when compared to that of the reference (no potentiator candidate), illustrating a difference in C. albicans susceptibility (Table 24):
Isoleucine (0.01%): resulted in a 4× increase in MBC when compared to that of the reference, and a 2× increase in MBC when compared to the enhancing molecule guaiacol, meaning that only 0.0000391% (v/v) instead of 0.000156% (v/v) GOX+LPO enzyme solution is needed to completely inhibit the growth of C. albicans. In other words, C. albicans showed to be more susceptible when isoleucine (0.01%) was added to the reaction mix containing 0.005%-0.0000391% (v/v) GOX+LPO enzyme solution.
Serine (0.01%): resulted in a 4× increase in MBC when compared to that of the reference, and a 2× increase in MBC when compared to the enhancing molecule guaiacol, meaning that only 0.0000391% (v/v) instead of 0.000156% (v/v) GOX+LPO enzyme solution is needed to completely inhibit the growth of C. albicans. In other words, C. albicans showed to be more susceptible when serine (0.01%) was added to the reaction mix containing 0.005%-0.0000391% (v/v) GOX+LPO enzyme solution.
   2.2.4. A. niger

Results indicated that addition of proline in a final concentration ranging from 1% to 0.01% (v/v) to the enzymatic GOX+LPO combination resulted in a difference in MBC when compared to that of the reference (no potentiator candidate), illustrating a difference in A. niger susceptibility (Table 25):
Proline (1%-0.01%): resulted in a 2× increase in MBC when compared to that of the reference, meaning that only 0.000156% (v/v) instead of 0.000313% (v/v) GOX+LPO enzyme solution is needed to completely inhibit the growth of A. niger. In other words, A. niger showed to be more susceptible when proline (1%-0.01%) was added to the reaction mix containing 0.005%-0.000156% (v/v) GOX+LPO enzyme solution.

Results indicated that addition of serine in a final concentration ranging from 1% to 0.01% (v/v) to the enzymatic GOX+LPO combination resulted in a difference in MBC when compared to that of the reference (no potentiator candidate), illustrating a difference in A. niger susceptibility (Table 26):
Serine (1%-0.01%): resulted in a 2× increase in MBC when compared to that of the reference, meaning that only 0.000156% (v/v) instead of 0.000313% (v/v) GOX+LPO enzyme solution is needed to completely inhibit the growth of A. niger. In other words, A. niger showed to be more susceptible when serine (1%-0.01%) was added to the reaction mix containing 0.005%-0.000156% (v/v) GOX+LPO enzyme solution.

Results indicated that addition of threonine in a final concentration ranging from 1% to 0.01% (v/v) to the enzymatic GOX+LPO combination resulted in a difference in MBC when compared to that of the reference (no potentiator candidate), illustrating a difference in A. niger susceptibility (Table 27):
Threonine (1%-0.01%): resulted in a 2× increase in MBC when compared to that of the reference, meaning that only 0.000156% (v/v) instead of 0.000313% (v/v) GOX+LPO enzyme solution is needed to completely inhibit the growth of A. niger. In other words, A. niger showed to be more susceptible when threonine (1%-0.01%) was added to the reaction mix containing 0.005%-0.000156% (v/v) GOX+LPO enzyme solution.

Results indicated that addition of glutamine in a final concentration ranging from 1% to 0.01% (v/v) to the enzymatic GOX+LPO combination resulted in a difference in MBC when compared to that of the reference (no potentiator candidate), illustrating a difference in A. niger susceptibility (Table 28):
Glutamine (1%-0.01%): resulted in a 2× increase in MBC when compared to that of the reference, meaning that only 0.000156% (v/v) instead of 0.000313% (v/v) GOX+LPO enzyme solution is needed to completely inhibit the growth of A. niger. In other words, A. niger showed to be more susceptible when glutamine (1%-0.01%) was added to the reaction mix containing 0.005%-0.000156% (v/v) GOX+LPO enzyme solution.

Results indicated that addition of asparagine in a final concentration ranging from 1% to 0.05% (v/v) to the enzymatic GOX+LPO combination resulted in a difference in MBC when compared to that of the reference (no potentiator candidate), illustrating a difference in A. niger susceptibility (Table 29):
Asparagine (1%-0.05%): resulted in a 2× increase in MBC when compared to that of the reference, meaning that only 0.000156% (v/v) instead of 0.000313% (v/v) GOX+LPO enzyme solution is needed to completely inhibit the growth of A. niger. In other words, A. niger showed to be more susceptible when asparagine (1%-0.05%) was added to the reaction mix containing 0.005%-0.000156% (v/v) GOX+LPO enzyme solution.

Results indicated that addition of isoleucine in a final concentration ranging from 1% to 0.005% (v/v) to the enzymatic GOX+LPO combination resulted in a difference in MBC when compared to that of the reference (no potentiator candidate), illustrating a difference in A. niger susceptibility (Table 30):
Isoleucine (1%-0.005%): resulted in a 2× increase in MIC when compared to that of the reference, meaning that only 0.000156% (v/v) instead of 0.000313% (v/v) GOX+LPO enzyme solution is needed to completely inhibit the growth of A. niger. In other words, A. niger showed to be more susceptible when isoleucine (1%-0.005%) was added to the reaction mix containing 0.005%-0.000156% (v/v) GOX+LPO enzyme solution.

### 2.3. Absolute Bacterial Counts

Absolute microbial counts in function of concentration of the antimicrobial agent tested are useful to monitor the bactericidal and fungicidal efficacy of that agent. Here, the antimicrobial agent is a combination of the natural enzymes GOX+LPO and absolute microbial counts (in function of enzyme solution concentration) were studied as such and when GOX+LPO was combined with a potentiator candidate (=a molecule that does not possess an antimicrobial effect as such, but is able to augment the antimicrobial effect of an antimicrobial agent).

To determine the absolute number of live microorganisms in function of different enzyme solution concentrations tested, a classical broth microdilution assay combined with standard CFU enumeration techniques was used to determine the susceptibility of a series of microbes commonly detected in infected wounds, including (a) the Gram-positive bacterium Staphylococcus aureus ATCC® 6538™ (results shown below), (b) the Gram-negative bacterium Pseudomonas aeruginosa ATCC®9027™ (results shown below), (c) the yeast Candida albicans ATCC®10231™ (results shown below), and (d) the fungus Aspergillus niger ATCC®16404™ (results shown below). In brief, the broth microdilution assay was set up as previously described (see 2.1). After incubation, the absolute microbial count at the MIC and following higher concentrations (MICx2, MICx4, and so on) was determined using standard CFU enumeration techniques. For each combination evaluated, this test was repeated at least 5 times. The collected data were further analyzed in MS Excel and expressed as log CFU/ml. Mean results were plotted in function of enzyme solution concentration.

2.3.1. S. aureus

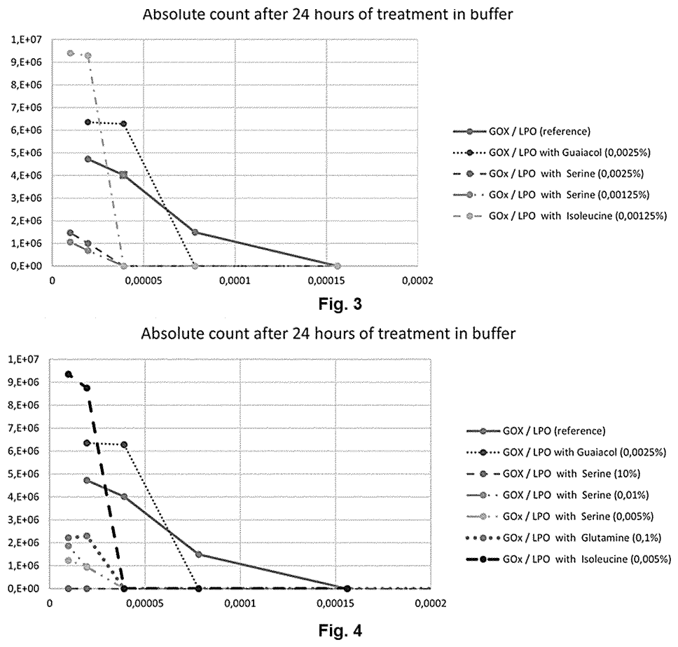

Results indicated that addition of serine and isoleucine in a final concentration ranging from 0.0025% to 0.00125% (v/v) to the enzymatic GOX+LPO combination resulted in a difference in absolute microbial counts when compared to that of the reference (no potentiator candidate), illustrating a difference in S. aureus susceptibility (FIG. 3):
Serine (0.0025-0.00125%): resulted in a stronger reduction in absolute S. aureus counts when compared to the that of the reference and when compared to the solution were guaiacol was added as enhancing agent, at a concentration of 0.005%-0.0000391% (v/v) GOX+LPO enzyme solution. In other words, S. aureus showed to be more susceptible when serine (0.0025%-0.00125%) was added to the reaction mix.
Isoleucine (0.00125%): resulted in a stronger reduction in absolute S. aureus counts when compared to the that of the reference and when compared to the solution were guaiacol was added as enhancing agent, at a concentration of 0.005%-0.0000391% (v/v) GOX+LPO enzyme solution. In other words, S. aureus showed to be more susceptible when serine (0.00125%) was added to the reaction mix.

When the concentrations of the enhancing molecules were increased, the next results are obtained (FIG. 4):
Serine (10-0.005%): resulted in a stronger reduction in absolute S. aureus counts when compared to the that of the reference and when compared to the solution were guaiacol was added as enhancing agent, at a concentration of 0.005%-0.0000391% (v/v) GOX+LPO enzyme solution. In other words, S. aureus showed to be more susceptible when serine (10%-0.005%) was added to the reaction mix.
Isoleucine (0.005%): resulted in a stronger reduction in absolute S. aureus counts when compared to the that of the reference and when compared to the solution were guaiacol was added as enhancing agent, at a concentration of 0.005%-0.0000391% (v/v) GOX+LPO enzyme solution. In other words, S. aureus showed to be more susceptible when serine (0.005%) was added to the reaction mix.
Glutamine (0.1%): resulted in a stronger reduction in absolute S. aureus counts when compared to the that of the reference and when compared to the solution were guaiacol was added as enhancing agent, at a concentration of 0.005%-0.0000391% (v/v) GOX+LPO enzyme solution. In other words, S. aureus showed to be more susceptible when glutamine (0.1%) was added to the reaction mix.
   2.3.2. P. aeruginosa

Results indicated that addition of serine and isoleucine in a final concentration ranging from 0.0025% to 0.00125% (v/v) to the enzymatic GOX+LPO combination resulted in a difference in absolute microbial counts when compared to that of the reference (no potentiator candidate), illustrating a difference in P. aeruginosa susceptibility (FIG. 5):
Serine (0.0025-0.00125%): resulted in a stronger reduction in absolute P. aeruginosa counts when compared to the that of the reference and when compared to the solution were guaiacol was added as enhancing agent, at a concentration of 0.005%-0.0000391% (v/v) GOX+LPO enzyme solution. In other words, P. aeruginosa showed to be more susceptible when serine (0.0025%-0.00125%) was added to the reaction mix.
Proline (0.0025-0.00125%): resulted in a stronger reduction in absolute P. aeruginosa counts when compared to the that of the reference and when compared to the solution were guaiacol was added as enhancing agent, at a concentration of 0.005%-0.0000391% (v/v) GOX+LPO enzyme solution. In other words, P. aeruginosa showed to be more susceptible when proline (0.0025-0.00125%) was added to the reaction mix.

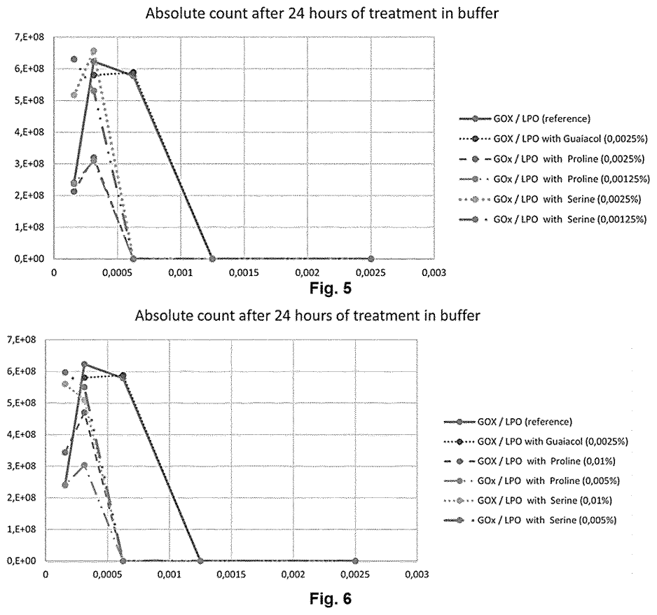

When the concentrations of the enhancing molecules were increased, the next results are obtained (FIG. 6):
Serine (0.01-0.005%): resulted in a stronger reduction in absolute P. aeruginosa counts when compared to the that of the reference and when compared to the solution were guaiacol was added as enhancing agent, at a concentration of 0.005%-0.0000391% (v/v) GOX+LPO enzyme solution. In other words, P. aeruginosa showed to be more susceptible when serine (0.01%-0.005%) was added to the reaction mix.
Proline (0.01-0.001%): resulted in a stronger reduction in absolute P. aeruginosa counts when compared to the that of the reference and when compared to the solution were guaiacol was added as enhancing agent, at a concentration of 0.005%-0.0000391% (v/v) GOX+LPO enzyme solution. In other words, P. aeruginosa showed to be more susceptible when proline (0.01-0.005%) was added to the reaction mix.

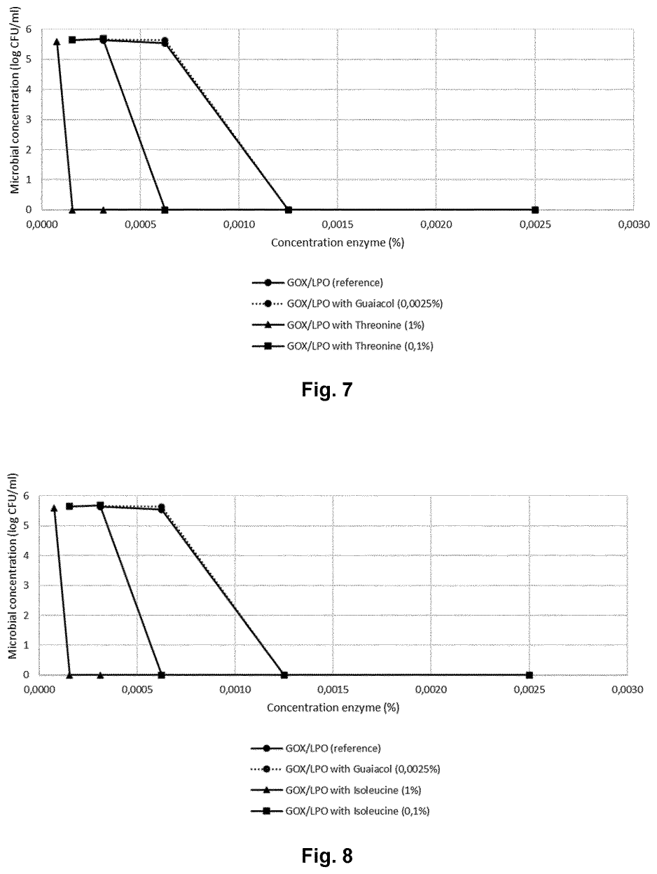

Results indicated that addition of threonine in a final concentration ranging from 1% to 0.1% (v/v to the enzymatic GOX+LPO combination resulted in a difference in absolute microbial counts when compared to that of the reference (no potentiator candidate), illustrating a difference in P. aeruginosa susceptibility (FIG. 7):
Threonine (1%): resulted in a stronger reduction in absolute P. aeruginosa counts when compared to the that of the reference at a concentration of 0,005%-0.000156% (v/v) GOX+LPO enzyme solution. In other words, P. aeruginosa showed to be more susceptible when threonine (1%) was added to the reaction mix.
Threonine (0,1%): resulted in a stronger reduction in absolute P. aeruginosa counts when compared to the that of the reference at a concentration of 0,005%-0.000625% (v/v) GOX+LPO enzyme solution. In other words, P. aeruginosa showed to be more susceptible when threonine (0,1%) was added to the reaction mix.

Results indicated that addition of isoleucine in a final concentration ranging from 10% to 0.1% (v/v to the enzymatic GOX+LPO combination resulted in a difference in absolute microbial counts when compared to that of the reference (no potentiator candidate), illustrating a difference in P. aeruginosa susceptibility (FIG. 8):
Isoleucine (1%): resulted in a stronger reduction in absolute P. aeruginosa counts when compared to the that of the reference at a concentration of 0,005%-0.000156% (v/v) GOX+LPO enzyme solution. In other words, P. aeruginosa showed to be more susceptible when isoleucine (1%) was added to the reaction mix.
Isoleucine (0,1%): resulted in a stronger reduction in absolute P. aeruginosa counts when compared to the that of the reference at a concentration of 0,005%-0.000625% (v/v) GOX+LPO enzyme solution. In other words, P. aeruginosa showed to be more susceptible when isoleucine (0,1%) was added to the reaction mix.

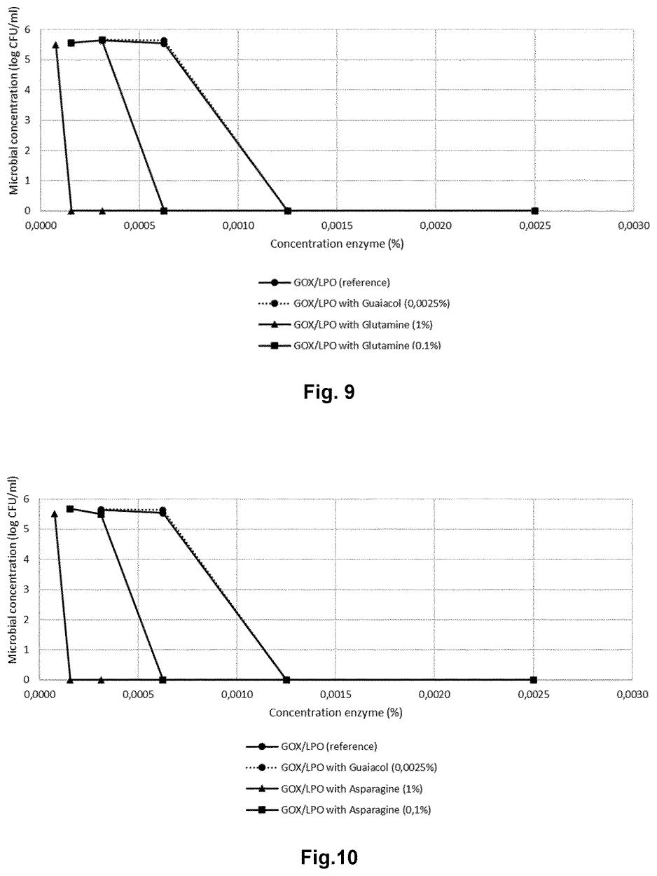

Results indicated that addition of glutamine in a final concentration ranging from 1% to 0.1% (v/v to the enzymatic GOX+LPO combination resulted in a difference in absolute microbial counts when compared to that of the reference (no potentiator candidate), illustrating a difference in P. aeruginosa susceptibility (FIG. 9):
Glutamine (1%): resulted in a stronger reduction in absolute P. aeruginosa counts when compared to the that of the reference at a concentration of 0,005%-0.000156% (v/v) GOX+LPO enzyme solution. In other words, P. aeruginosa showed to be more susceptible when glutamine (1%) was added to the reaction mix.
Glutamine (0,1%): resulted in a stronger reduction in absolute P. aeruginosa counts when compared to the that of the reference at a concentration of 0,005%-0.000625% (v/v) GOX+LPO enzyme solution. In other words, P. aeruginosa showed to be more susceptible when glutamine (0,1%) was added to the reaction mix.

Results indicated that addition of asparagine in a final concentration ranging from 1% to 0.1% (v/v to the enzymatic GOX+LPO combination resulted in a difference in absolute microbial counts when compared to that of the reference (no potentiator candidate), illustrating a difference in P. aeruginosa susceptibility (FIG. 10):
Asparagine (1%): resulted in a stronger reduction in absolute P. aeruginosa counts when compared to the that of the reference at a concentration of 0,005%-0.000156% (v/v) GOX+LPO enzyme solution. In other words, P. aeruginosa showed to be more susceptible when asparagine (1%) was added to the reaction mix.
Asparagine (0,1%): resulted in a stronger reduction in absolute P. aeruginosa counts when compared to the that of the reference at a concentration of 0,005%-0.000625% (v/v) GOX+LPO enzyme solution. In other words, P. aeruginosa showed to be more susceptible when asparagine (0,1%) was added to the reaction mix.
   2.3.3. C. albicans

Results indicated that addition of serine and isoleucine in a final concentration ranging from 0.0025% to 0.00125% (v/v) to the enzymatic GOX+LPO combination resulted in a difference in absolute microbial counts when compared to that of the reference (no potentiator candidate), illustrating a difference in C. albicans susceptibility (FIG. 11):
Serine (0.01%): resulted in a stronger reduction in absolute C. albicans counts when compared to the that of the reference and when compared to the solution were guaiacol was added at a concentration of 0.005%-0.0000391% (v/v) GOX+LPO enzyme solution. In other words, C. albicans showed to be more susceptible when serine (0.01%) was added to the reaction mix.
Isoleucine (0.01%): resulted in a stronger reduction in absolute C. albicans counts when compared to the that of the reference and when compared to the solution were guaiacol was added at a concentration of 0.005%-0.0000391% (v/v) GOX+LPO enzyme solution. In other words, C. albicans showed to be more susceptible when proline (0.01%) was added to the reaction mix.
   2.3.4. A. niger

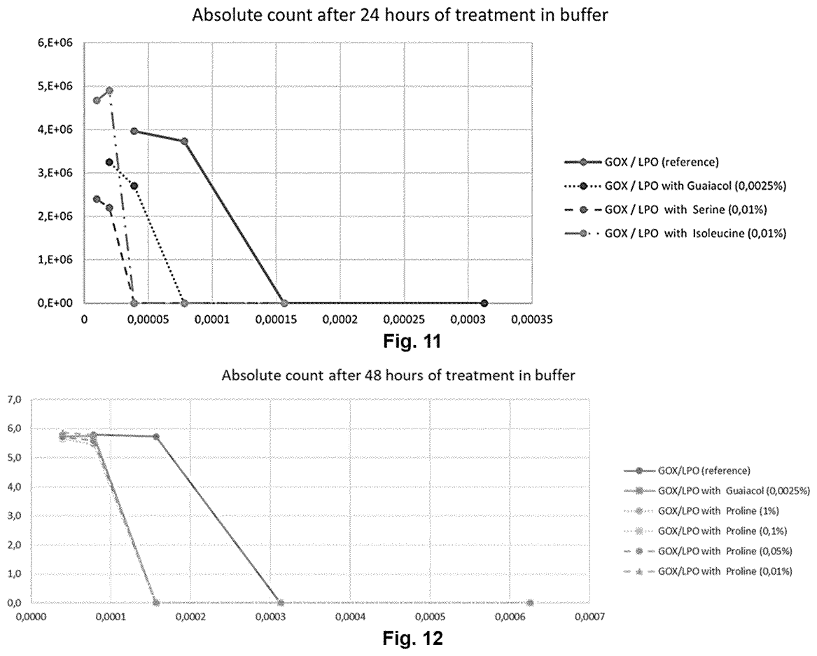

Results indicated that addition of proline in a final concentration ranging from 1% to 0.01% (v/v to the enzymatic GOX+LPO combination resulted in a difference in absolute microbial counts when compared to that of the reference (no potentiator candidate), illustrating a difference in A. niger susceptibility (FIG. 12):
Proline (1-0.01%): resulted in a stronger reduction in absolute A. niger counts when compared to the that of the reference at a concentration of 0.005%-0.000156% (v/v) GOX+LPO enzyme solution. In other words, A. niger showed to be more susceptible when proline (1%-0.01%) was added to the reaction mix.

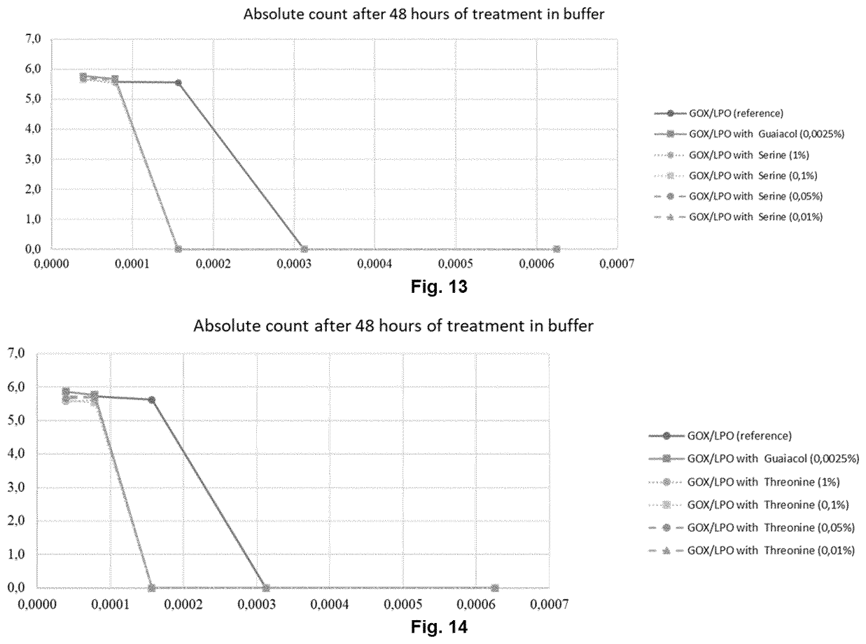

Results indicated that addition of serine in a final concentration ranging from 1% to 0.01% (v/v to the enzymatic GOX+LPO combination resulted in a difference in absolute microbial counts when compared to that of the reference (no potentiator candidate), illustrating a difference in A. niger susceptibility (FIG. 13):
Serine (1-0.010%): resulted in a stronger reduction in absolute A. niger counts when compared to the that of the reference at a concentration of 0.005%-0.000156% (v/v) GOX+LPO enzyme solution. In other words, A. niger showed to be more susceptible when serine (1%-0.01%) was added to the reaction mix.

Results indicated that addition of threonine in a final concentration ranging from 1% to 0.01% (v/v to the enzymatic GOX+LPO combination resulted in a difference in absolute microbial counts when compared to that of the reference (no potentiator candidate), illustrating a difference in A. niger susceptibility (FIG. 14):
Threonine (1-0.01%): resulted in a stronger reduction in absolute A. niger counts when compared to the that of the reference at a concentration of 0.005%-0.000156% (v/v) GOX+LPO enzyme solution. In other words, A. niger showed to be more susceptible when threonine (10%-0.010%) was added to the reaction mix.

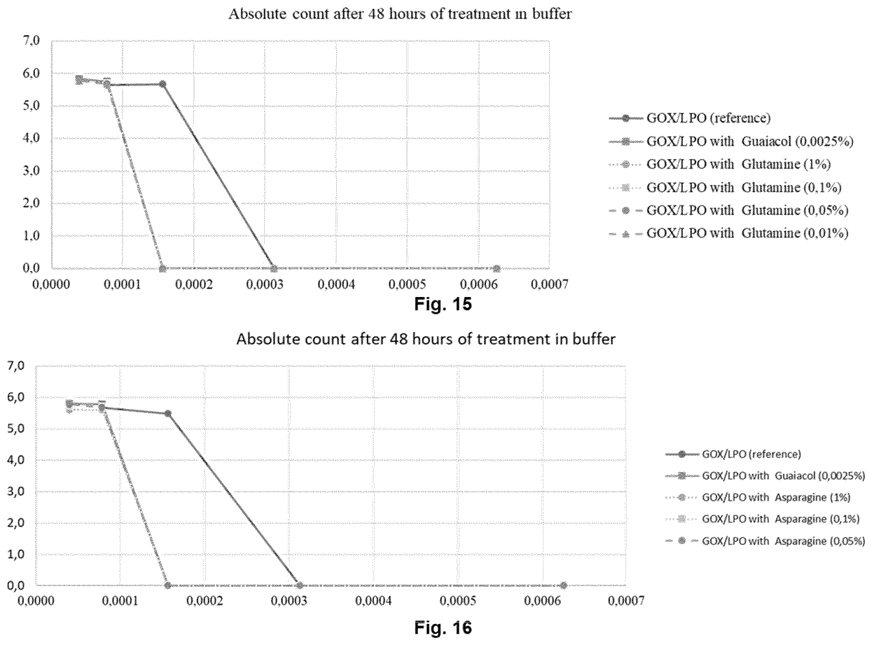

Results indicated that addition of glutamine in a final concentration ranging from 1% to 0.01% (v/v to the enzymatic GOX+LPO combination resulted in a difference in absolute microbial counts when compared to that of the reference (no potentiator candidate), illustrating a difference in A. niger susceptibility (FIG. 15):
Glutamine (1-0.01%): resulted in a stronger reduction in absolute A. niger counts when compared to the that of the reference at a concentration of 0.005%-0.000156% (v/v) GOX+LPO enzyme solution. In other words, A. niger showed to be more susceptible when glutamine (1%-0.01%) was added to the reaction mix.

Results indicated that addition of asparagine in a final concentration ranging from 1% to 0.05% (v/v to the enzymatic GOX+LPO combination resulted in a difference in absolute microbial counts when compared to that of the reference (no potentiator candidate), illustrating a difference in A. niger susceptibility (FIG. 16):
Asparagine (1-0.05%): resulted in a stronger reduction in absolute A. niger counts when compared to the that of the reference at a concentration of 0.005%-0.000156% (v/v) GOX+LPO enzyme solution. In other words, A. niger showed to be more susceptible when asparagine (1%-0.05%) was added to the reaction mix.

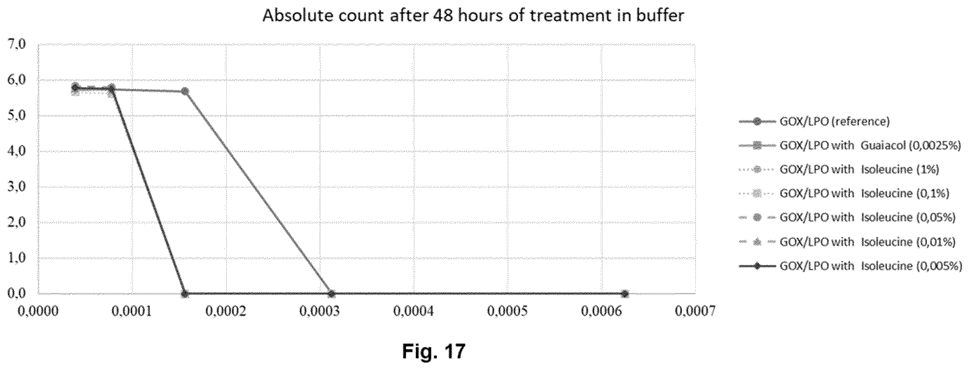

Results indicated that addition of isoleucine in a final concentration ranging from 1% to 0.005% (v/v to the enzymatic GOX+LPO combination resulted in a difference in absolute microbial counts when compared to that of the reference (no potentiator candidate), illustrating a difference in A. niger susceptibility (FIG. 17):
Isoleucine (1-0.005%): resulted in a stronger reduction in absolute A. niger counts when compared to the that of the reference at a concentration of 0.005%-0.000156% (v/v) GOX+LPO enzyme solution. In other words, A. niger showed to be more susceptible when isoleucine (1%-0.005%) was added to the reaction mix.

### 2.4. Challenge Testing in Alginogel Environment

To determine the ability of an antimicrobial agent to elicit an antimicrobial preserving efficacy in a gel environment, a microbiological challenge test can be performed. Here, the antimicrobial agent is a combination of the natural enzymes GOX+LPO. The antimicrobial preserving activity in gel environment of this enzyme combination GOX+LPO was studied as such and when combined with an enhancing agent (=a molecule that does not possess an antimicrobial effect as such, but is able to augment the antimicrobial effect of an antimicrobial agent). Microbial challenge testing was performed according to the method described in the European Pharmacopoeia current version chapter 5.1.3 ‘Efficacy of antimicrobial preservation’, but with adapted time points (0, 10, 20, 30, 40, 50, 60, 70 and 80 min instead of 0, 2, 7, 14 and 28 days). In brief, for every enhancing agent to be evaluated, a lab-scale alginogel (based on the Flaminal® formulation, but without the original enhancer guaiacol) including 0.0075% GOX+LPO (1:1) was prepared according to standard practices. Enhancing agents were added to the alginogel in a final concentration ranging from 0.01 to 0,00025% (v/w). As a reference, an alginogel including 0.0075% GOX+LPO (1:1) without the addition of any enhancing agent was included (designated as: enzymes only). As a control, an alginogel without both GOX+LPO (1:1) enzyme solution and enhancing agent (designated as: empty gel) as well as an alginogel only containing the enhancing agent only (designated as: enhancing candidate only) were included. After preparation, a homogenous sample of 10 g was transferred into a 50 ml conical tube for each alginogel to be evaluated and microorganism to be tested. For each test microorganism, including S. aureus (ATCC®6538™), P. aeruginosa (ATCC®9027™), C. albicans (ATCC®10231™) and A. niger (ATCC®16404 ™), a microbial suspension of about 108 CFU/ml was prepared in sterile diluent (buffered sodium chloride-peptone solution). Then, the 50 ml conical tubes containing the alginogel samples to be evaluated were inoculated with 100 μl of the designated inoculum. At the start (time point 0) and after 0, 10, 20, 30, 40, 50, 60, 70 and 80 min, the number of viable microorganisms per gram product (expressed as CFU/g) was determined by transferring a sample of 0.5 g of the product into a 15 ml conical tube containing 4.5 ml of sterile diluent and applying general dilution and plate count techniques. For each combination evaluated, this test was repeated at least 1-3 times. The collected data were further analyzed in MS Excel. Mean absolute counts (expressed as log CFU/g) were visualized in a graph.

2.4.1. Proline (S. aureus)

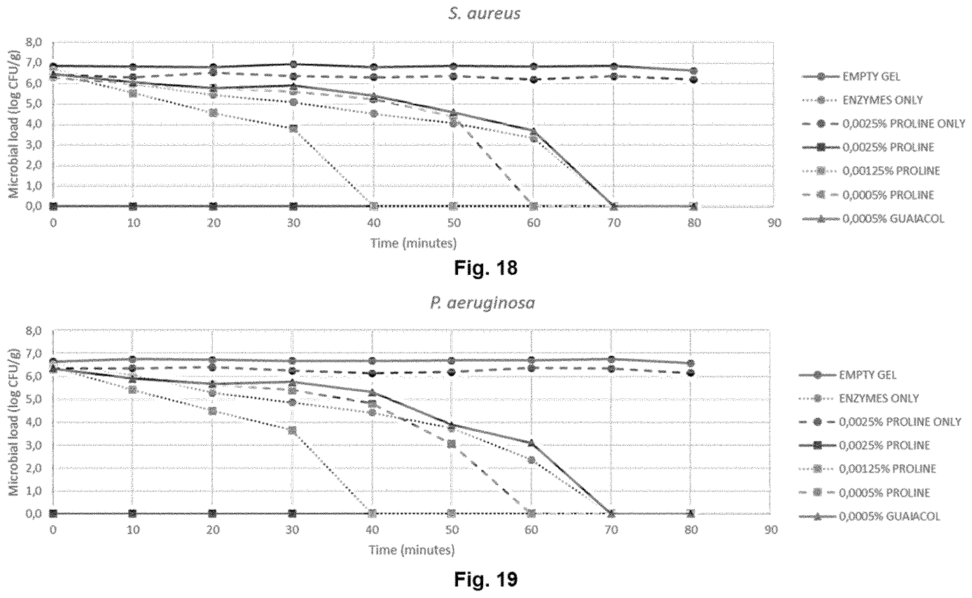

Results indicated that addition of proline in a final concentration ranging from 0.0025% to 0.0005% (v/v) to the enzymatic GOX+LPO combination resulted in a difference in antimicrobial efficacy when compared to that of the reference (i.e., enzymes only), illustrating a difference in S. aureus susceptibility (FIG. 18):
Proline (0.0025%): resulted in an immediate entire reduction in absolute S. aureus counts when compared to the that of the reference in a 0.0075% (v/v) GOX+LPO enzyme solution concentration. In other words, S. aureus showed to be most susceptible when proline (0.0025%) was added to the alginogel.
Proline (0.00125-0.0005%): resulted in a faster complete reduction in absolute S. aureus counts when compared to the that of the reference in a 0.0075% (v/v) GOX+LPO enzyme solution concentration. In other words, S. aureus showed to be more susceptible when proline (0.00125%-0.0005%) was added to the alginogel.

Further, it was indicated that the addition of proline in a final concentration of 0.0005% resulted in a faster complete reduction in absolute S. aureus counts compared to when guaiacol was added in the same concentration (i.e., 0.0005%). In other words, S. aureus showed to be more susceptible to the alginogel including the enzymatic GOX+LPO combination and proline than that including guaiacol.

Finally, there was no antimicrobial activity when S. aureus was exposed to the empty gel and enhancing candidate only (tested at highest concentration). This verifies that the alginogel as such does not possess any antimicrobial activity and that proline does not possess an antimicrobial effect as such, but is able to augment the antimicrobial effect of the GOX+LPO enzyme solution instead.

2.4.2. Proline (P. aeruginosa)

Results indicated that addition of proline in a final concentration ranging from 0.0025% to 0.0005% (v/v) to the enzymatic GOX+LPO combination resulted in a difference in absolute microbial counts when compared to that of the reference (no potentiator candidate), illustrating a difference in P. aeruginosa susceptibility (FIG. 19):
Proline (0.0025%): resulted in an immediate entire reduction in absolute P. aeruginosa counts when compared to the that of the reference in a 0.0075% (v/v) GOX+LPO enzyme solution concentration. In other words, P. aeruginosa showed to be most susceptible when proline (0.0025%) was added to the alginogel.
Proline (0.00125-0.0005%): resulted in a faster complete reduction in absolute P. aeruginosa counts when compared to the that of the reference in a 0.0075% (v/v) GOX+LPO enzyme solution concentration. In other words P. aeruginosa showed to be more susceptible when proline (0.00125%-0.0005%) was added to the alginogel.

Further, it was indicated that the addition of proline in a final concentration of 0.0005% resulted in a faster complete reduction in absolute P. aeruginosa counts compared to when guaiacol was added in the same concentration (i.e., 0.0005%). In other words P. aeruginosa showed to be more susceptible to the alginogel including the enzymatic GOX+LPO combination and proline than that including guaiacol.

Finally, there was no antimicrobial activity when P. aeruginosa was exposed to the empty gel and enhancing candidate only (tested at highest concentration). This verifies that the alginogel as such does not possess any antimicrobial activity and that proline does not possess an antimicrobial effect as such, but is able to augment the antimicrobial effect of the GOX+LPO enzyme solution instead.

2.4.3. Proline (C. albicans)

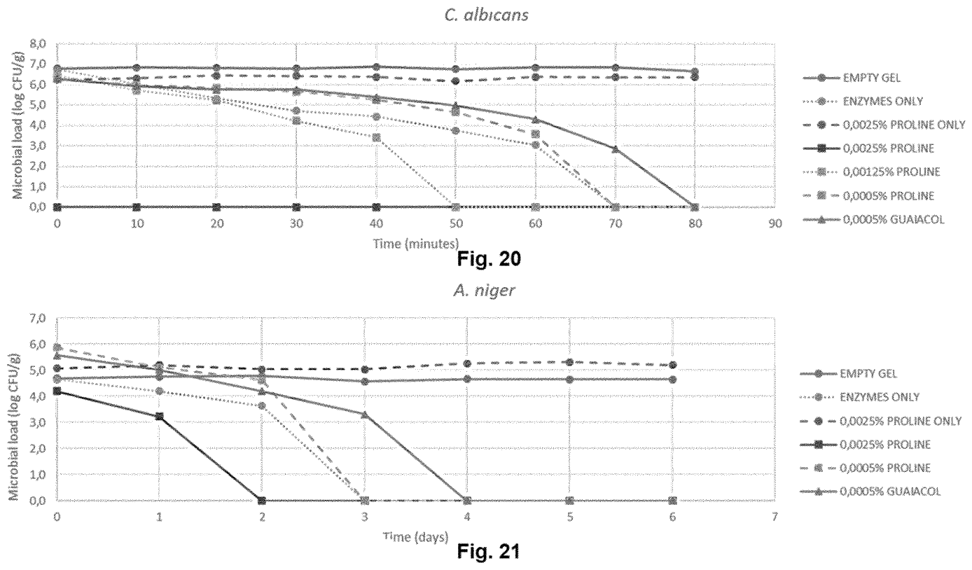

Results indicated that addition of proline in a final concentration ranging from 0.0025% to 0.0005% (v/v) to the enzymatic GOX+LPO combination resulted in a difference in absolute microbial counts when compared to that of the reference (no potentiator candidate), illustrating a difference in C. albicans susceptibility (FIG. 20):
Proline (0.0025%): resulted in an immediate entire reduction in absolute C. albicans counts when compared to the that of the reference in a 0.0075% (v/v) GOX+LPO enzyme solution concentration. In other words, C. albicans showed to be most susceptible when proline (0.0025%) was added to the alginogel.
Proline (0.00125%): resulted in a faster complete reduction in absolute C. albicans counts when compared to the that of the reference in a 0.0075% (v/v) GOX+LPO enzyme solution concentration. In other words C. albicans showed to be more susceptible when proline (0.00125%) was added to the alginogel.

Further, it was indicated that the addition of proline in a final concentration of 0.0005% resulted in a faster complete reduction in absolute C. albicans counts compared to when guaiacol was added in the same concentration (i.e., 0.0005%). In other words C. albicans showed to be more susceptible to the alginogel including the enzymatic GOX+LPO combination and proline than that including guaiacol.

Finally, there was no antimicrobial activity when C. albicans was exposed to the empty gel and enhancing candidate only (tested at highest concentration). This verifies that the alginogel as such does not possess any antimicrobial activity and that proline does not possess an antimicrobial effect as such, but is able to augment the antimicrobial effect of the GOX+LPO enzyme solution instead.

2.4.4. Proline (A. niger)

Results indicated that addition of proline in a final concentration ranging from 0.0025% (v/v) to the enzymatic GOX+LPO combination resulted in a difference in absolute microbial counts when compared to that of the reference (no potentiator candidate), illustrating a difference in A. niger susceptibility (FIG. 21):
Proline (0.0025%): resulted in a faster complete reduction in absolute A. niger counts when compared to the that of the reference in a 0.0075% (v/v) GOX+LPO enzyme solution concentration. In other words A. niger showed to be more susceptible when proline (0.0025%) was added to the alginogel.

Further, it was indicated that the addition of proline in a final concentration of 0.0005% resulted in a faster complete reduction in absolute A. niger counts compared to when guaiacol was added in the same concentration (i.e., 0.0005%). In other words A. niger showed to be more susceptible to the alginogel including the enzymatic GOX+LPO combination and proline than that including guaiacol.

Finally, there was no antimicrobial activity when A. niger was exposed to the empty gel and enhancing candidate only (tested at highest concentration). This verifies that the alginogel as such does not possess any antimicrobial activity and that proline does not possess an antimicrobial effect as such, but is able to augment the antimicrobial effect of the GOX+LPO enzyme solution instead.

2.4.5. Serine (S. aureus)

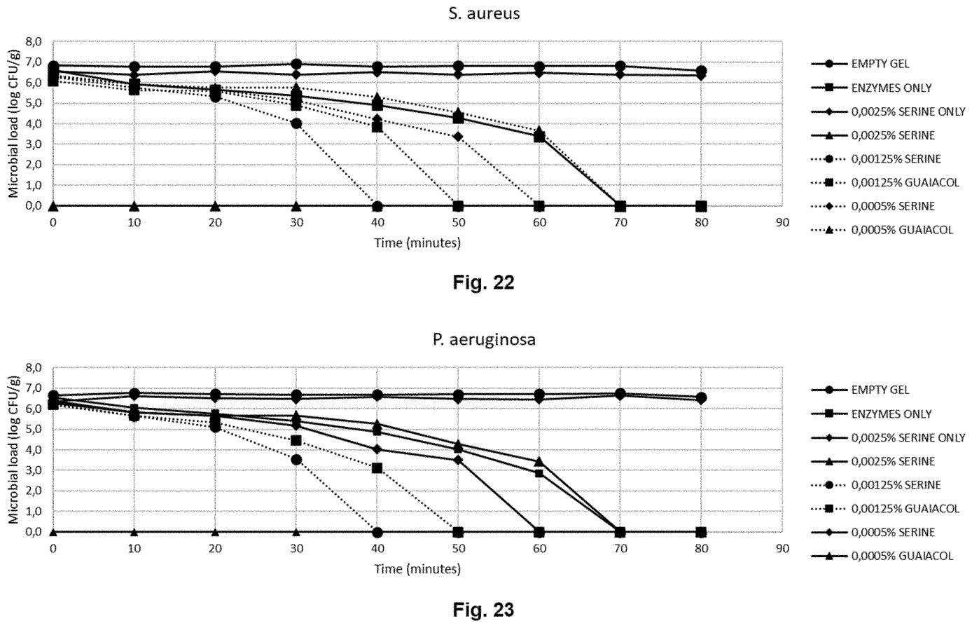

Results indicated that addition of serine in a final concentration ranging from 0,0025 to 0,0005% (v/v) to the enzymatic GOX+LPO combination resulted in a difference in antimicrobial efficacy when compared to that of the reference (i.e., enzymes only), illustrating a difference in S. aureus susceptibility (FIG. 22):
Serine (0,0025%): resulted in an immediate entire reduction in absolute S. aureus counts when compared to the that of the reference in a 0,0075% (v/v) GOX+LPO enzyme solution concentration. In other words, S. aureus showed to be most susceptible when serine (0,0025%) was added to the alginogel.
Serine (0,00125-0,0005%): resulted in a faster complete reduction in absolute S. aureus counts when compared to the that of the reference in a 0,0075% (v/v) GOX+LPO enzyme solution concentration. In other words, S. aureus showed to be more susceptible when serine (0,00125-0,0005%) was added to the alginogel.

Further, it was indicated that the addition of serine in a final concentration of 0.0005% resulted in a faster complete reduction in absolute S. aureus counts compared to when guaiacol was added in the same concentration (i.e., 0.0005%). In other words, S. aureus showed to be more susceptible to the alginogel including the enzymatic GOX+LPO combination and serine than that including guaiacol.

Finally, there was no antimicrobial activity when S. aureus was exposed to the empty gel and enhancing candidate only (tested at highest concentration). This verifies that the alginogel as such does not possess any antimicrobial activity and that serine does not possess an antimicrobial effect as such, but is able to augment the antimicrobial effect of the GOX+LPO enzyme solution instead.

2.4.6. Serine (P. aeruginosa)

Results indicated that addition of serine in a final concentration ranging from 0,0025 to 0,0005% (v/v) to the enzymatic GOX+LPO combination resulted in a difference in absolute microbial counts when compared to that of the reference (no potentiator candidate), illustrating a difference in P. aeruginosa susceptibility (FIG. 23):
Serine (0,0025%): resulted in an immediate entire reduction in absolute P. aeruginosa counts when compared to the that of the reference in a 0,0075% (v/v) GOX+LPO enzyme solution concentration. In other words, P. aeruginosa showed to be most susceptible when serine (0,0025%) was added to the alginogel.
Serine (0,00125-0,0005%): resulted in a faster complete reduction in absolute P. aeruginosa counts when compared to the that of the reference in a 0,0075% (v/v) GOX+LPO enzyme solution concentration. In other words, P. aeruginosa showed to be more susceptible when serine (0,00125-0,0005%) was added to the alginogel.

Further, it was indicated that the addition of serine in a final concentration of 0.0005% resulted in a faster complete reduction in absolute P. aeruginosa counts compared to when guaiacol was added in the same concentration (i.e., 0.0005%). In other words, P. aeruginosa showed to be more susceptible to the alginogel including the enzymatic GOX+LPO combination and serine than that including guaiacol.

Finally, there was no antimicrobial activity when P. aeruginosa was exposed to the empty gel and enhancing candidate only (tested at highest concentration). This verifies that the alginogel as such does not possess any antimicrobial activity and that serine does not possess an antimicrobial effect as such, but is able to augment the antimicrobial effect of the GOX+LPO enzyme solution instead.

2.4.7. Serine (C. albicans)

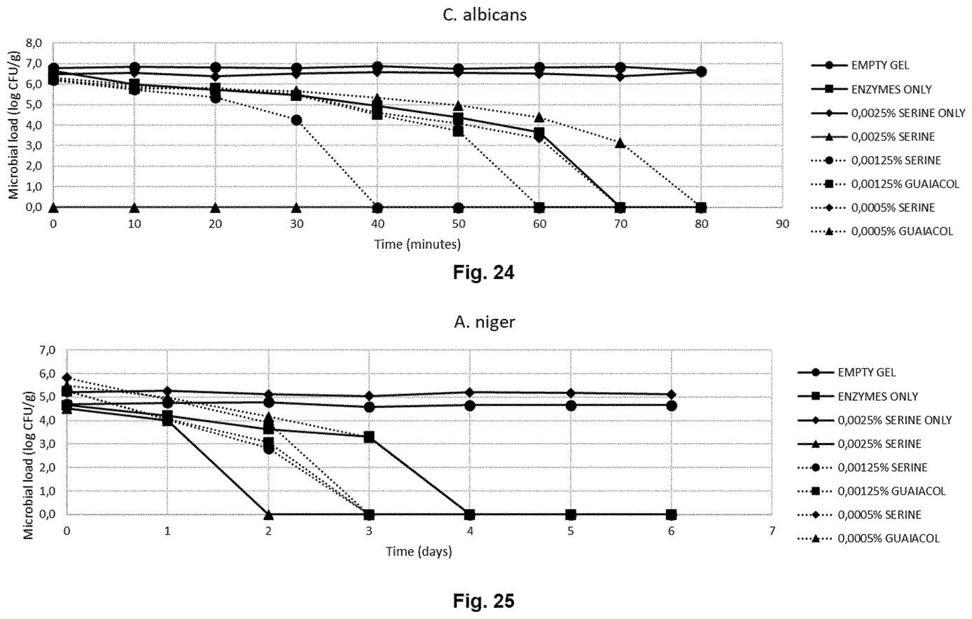

Results indicated that addition of serine in a final concentration ranging from 0,0025 to 0,0005% (v/v) to the enzymatic GOX+LPO combination resulted in a difference in absolute microbial counts when compared to that of the reference (no potentiator candidate), illustrating a difference in C. albicans susceptibility (FIG. 24):
Serine (0,0025%): resulted in an immediate entire reduction in absolute C. albicans counts when compared to the that of the reference in a 0,0075% (v/v) GOX+LPO enzyme solution concentration. In other words, C. albicans showed to be most susceptible when serine (0,0025%) was added to the alginogel.
Serine (0,00125%): resulted in a faster complete reduction in absolute C. albicans counts when compared to the that of the reference in a 0,0075% (v/v) GOX+LPO enzyme solution concentration. In other words, C. albicans showed to be more susceptible when serine (0,00125%) was added to the alginogel.

Further, it was indicated that the addition of serine in a final concentration of 0.0005% resulted in a faster complete reduction in absolute C. albicans counts compared to when guaiacol was added in the same concentration (i.e., 0.0005%). In other words, P. aeruginosa showed to be more susceptible to the alginogel including the enzymatic GOX+LPO combination and serine than that including guaiacol.

Finally, there was no antimicrobial activity when C. albicans was exposed to the empty gel and enhancing candidate only (tested at highest concentration). This verifies that the alginogel as such does not possess any antimicrobial activity and that serine does not possess an antimicrobial effect as such, but is able to augment the antimicrobial effect of the GOX+LPO enzyme solution instead.

2.4.8. Serine (A. niger)

Results indicated that addition of serine in a final concentration ranging from 0,0025 to 0,0005% (v/v) to the enzymatic GOX+LPO combination resulted in a difference in absolute microbial counts when compared to that of the reference (no potentiator candidate), illustrating a difference in A. niger susceptibility (FIG. 25):
Serine (0,0025-0,0005%): resulted in a faster complete reduction in absolute A. niger counts when compared to the that of the reference in a 0,0075% (v/v) GOX+LPO enzyme solution concentration. In other words, A. niger showed to be more susceptible when serine (0,0025-0,0005%) was added to the alginogel.

Further, it was indicated that the addition of serine in a final concentration of 0.0005% resulted in a faster complete reduction in absolute A. niger counts compared to when guaiacol was added in the same concentration (i.e., 0.0005%). In other words, A. niger showed to be more susceptible to the alginogel including the enzymatic GOX+LPO combination and serine than that including guaiacol.

Finally, there was no antimicrobial activity when A. niger was exposed to the empty gel and enhancing candidate only (tested at highest concentration). This verifies that the alginogel as such does not possess any antimicrobial activity and that serine does not possess an antimicrobial effect as such, but is able to augment the antimicrobial effect of the GOX+LPO enzyme solution instead.

2.4.9. Threonine (S. aureus)

Results indicated that addition of threonine in a final concentration ranging from 0,0025 to 0,0005% (v/v) to the enzymatic GOX+LPO combination resulted in a difference in antimicrobial efficacy when compared to that of the reference (i.e., enzymes only), illustrating a difference in S. aureus susceptibility (FIG. 26):
Threonine (0,0025%): resulted in an immediate entire reduction in absolute S. aureus counts when compared to the that of the reference in a 0,0075% (v/v) GOX+LPO enzyme solution concentration. In other words, S. aureus showed to be most susceptible when threonine (0,0025%) was added to the alginogel.
Threonine (0,00125-0,0005%): resulted in a faster complete reduction in absolute S. aureus counts when compared to the that of the reference in a 0,0075% (v/v) GOX+LPO enzyme solution concentration. In other words, S. aureus showed to be more susceptible when threonine (0,00125-0,0005%) was added to the alginogel.

Further, it was indicated that the addition of threonine in a final concentration of 0.0005% resulted in a faster complete reduction in absolute S. aureus counts compared to when guaiacol was added in the same concentration (i.e., 0.0005%). In other words, S. aureus showed to be more susceptible to the alginogel including the enzymatic GOX+LPO combination and threonine than that including guaiacol.

Finally, there was no antimicrobial activity when S. aureus was exposed to the empty gel and enhancing candidate only (tested at highest concentration). This verifies that the alginogel as such does not possess any antimicrobial activity and that threonine does not possess an antimicrobial effect as such, but is able to augment the antimicrobial effect of the GOX+LPO enzyme solution instead.

2.4.10. Threonine (P. aeruginosa)

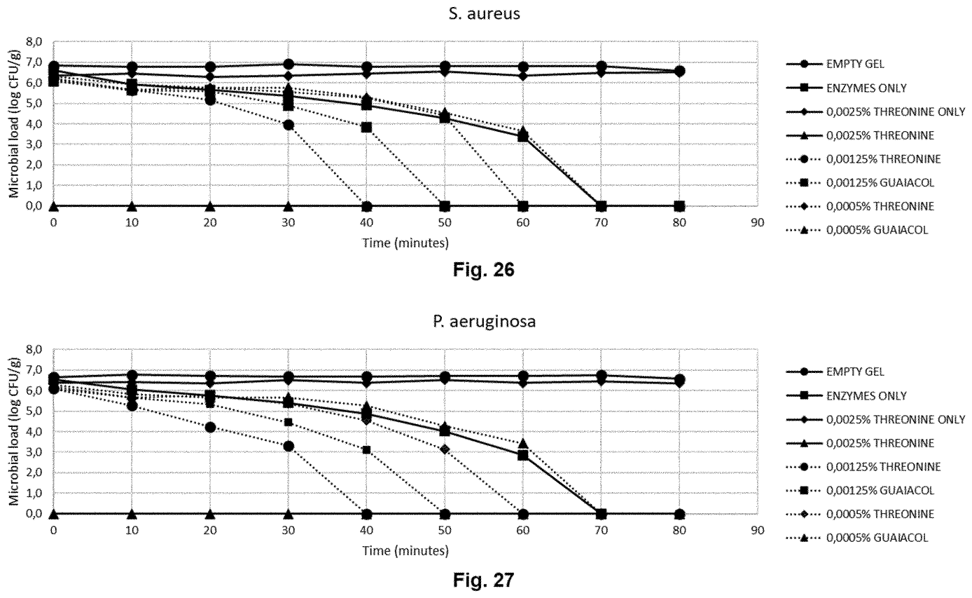

Results indicated that addition of threonine in a final concentration of 0,0025% to 0,0005% (v/v) to the enzymatic GOX+LPO combination resulted in a difference in absolute microbial counts when compared to that of the reference (no potentiator candidate), illustrating a difference in P. aeruginosa susceptibility (FIG. 27):
Threonine (0,0025%): resulted in an immediate entire reduction in absolute P. aeruginosa counts when compared to the that of the reference in a 0,0075% (v/v) GOX+LPO enzyme solution concentration. In other words, P. aeruginosa showed to be most susceptible when threonine (0,0025%) was added to the alginogel.
Threonine (0,00125-0,0005%): resulted in a faster complete reduction in absolute P. aeruginosa counts when compared to the that of the reference in a 0,0075% (v/v) GOX+LPO enzyme solution concentration. In other words, P. aeruginosa showed to be more susceptible when threonine (0,00125-0,0005%) was added to the alginogel.

Further, it was indicated that the addition of threonine in a final concentration of 0.0005% resulted in a faster complete reduction in absolute P. aeruginosa counts compared to when guaiacol was added in the same concentration (i.e., 0.0005%). In other words, P. aeruginosa showed to be more susceptible to the alginogel including the enzymatic GOX+LPO combination and threonine than that including guaiacol.

Finally, there was no antimicrobial activity when P. aeruginosa was exposed to the empty gel and enhancing candidate only (tested at highest concentration). This verifies that the alginogel as such does not possess any antimicrobial activity and that threonine does not possess an antimicrobial effect as such, but is able to augment the antimicrobial effect of the GOX+LPO enzyme solution instead.

2.4.11. Threonine (C. albicans)

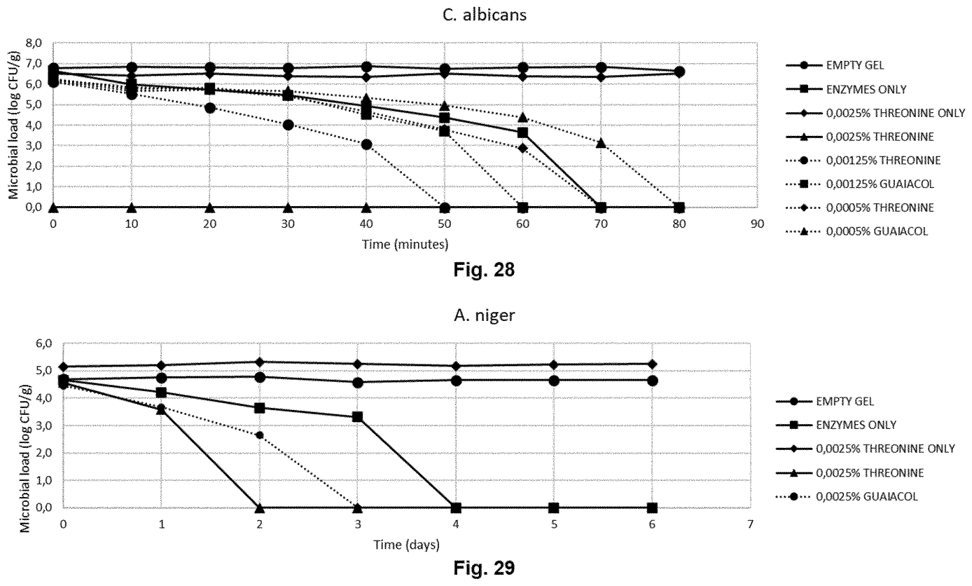

Results indicated that addition of threonine in a final concentration ranging from 0,0025 to 0,0005% (v/v) to the enzymatic GOX+LPO combination resulted in a difference in absolute microbial counts when compared to that of the reference (no potentiator candidate), illustrating a difference in C. albicans susceptibility (FIG. 28):
Threonine (0,0025%): resulted in an immediate entire reduction in absolute C. albicans counts when compared to the that of the reference in a 0,0075% (v/v) GOX+LPO enzyme solution concentration. In other words, C. albicans showed to be most susceptible when threonine (0,0025%) was added to the alginogel.
Threonine (0,00125-0,0005%): resulted in a faster complete reduction in absolute C. albicans counts when compared to the that of the reference in a 0,0075% (v/v) GOX+LPO enzyme solution concentration. In other words, C. albicans showed to be more susceptible when threonine (0,00125-0,0005%) was added to the alginogel.

Further, it was indicated that the addition of threonine in a final concentration of 0.0005% resulted in a faster complete reduction in absolute C. albicans counts compared to when guaiacol was added in the same concentration (i.e., 0.0005%). In other words, C. albicans showed to be more susceptible to the alginogel including the enzymatic GOX+LPO combination and threonine than that including guaiacol.

Finally, there was no antimicrobial activity when C. albicans was exposed to the empty gel and enhancing candidate only (tested at highest concentration). This verifies that the alginogel as such does not possess any antimicrobial activity and that threonine does not possess an antimicrobial effect as such, but is able to augment the antimicrobial effect of the GOX+LPO enzyme solution instead.

2.4.12. Threonine (A. niger)

Results indicated that addition of threonine in a final concentration of 0,0025% (v/v) to the enzymatic GOX+LPO combination resulted in a difference in absolute microbial counts when compared to that of the reference (no potentiator candidate), illustrating a difference in A. niger susceptibility (FIG. 29):
Threonine (0,0025%): resulted in a faster complete reduction in absolute A. niger counts when compared to the that of the reference in a 0,0075% (v/v) GOX+LPO enzyme solution concentration. In other words, A. niger showed to be more susceptible when threonine (0,0025%) was added to the alginogel.

Further, it was indicated that the addition of threonine in a final concentration of 0.0025% resulted in a faster complete reduction in absolute A. niger counts compared to when guaiacol was added in the same concentration (i.e., 0.0025%). In other words, A. niger showed to be more susceptible to the alginogel including the enzymatic GOX+LPO combination and threonine than that including guaiacol.

Finally, there was no antimicrobial activity when A. niger was exposed to the empty gel and enhancing candidate only (tested at highest concentration). This verifies that the alginogel as such does not possess any antimicrobial activity and that threonine does not possess an antimicrobial effect as such, but is able to augment the antimicrobial effect of the GOX+LPO enzyme solution instead.

2.4.13. Glutamine (S. aureus)

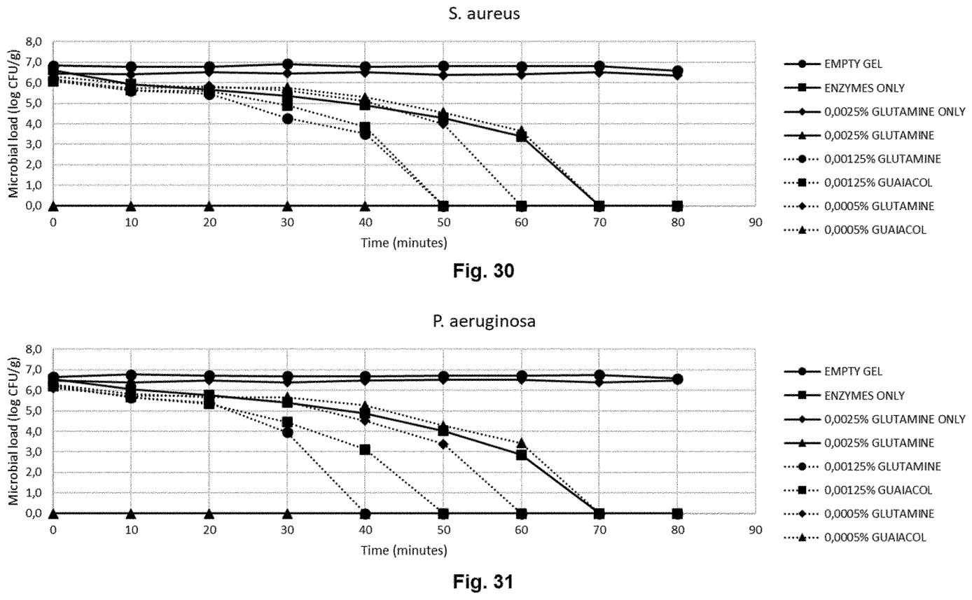

Results indicated that addition of glutamine in a final concentration ranging from 0,0025 to 0,0005% (v/v) to the enzymatic GOX+LPO combination resulted in a difference in antimicrobial efficacy when compared to that of the reference (i.e., enzymes only), illustrating a difference in S. aureus susceptibility (FIG. 30):
Glutamine (0,0025%): resulted in an immediate entire reduction in absolute S. aureus counts when compared to the that of the reference in a 0,0075% (v/v) GOX+LPO enzyme solution concentration. In other words, S. aureus showed to be most susceptible when glutamine (0,0025%) was added to the alginogel.
Glutamine (0,00125-0,0005%): resulted in a faster complete reduction in absolute S. aureus counts when compared to the that of the reference in a 0,0075% (v/v) GOX+LPO enzyme solution concentration. In other words, S. aureus showed to be more susceptible when glutamine (0,00125-0,0005%) was added to the alginogel.

Further, it was indicated that the addition of glutamine in a final concentration of 0.0005% resulted in a faster complete reduction in absolute S. aureus counts compared to when guaiacol was added in the same concentration (i.e., 0.0005%). In other words, S. aureus showed to be more susceptible to the alginogel including the enzymatic GOX+LPO combination and glutamine than that including guaiacol.

Finally, there was no antimicrobial activity when S. aureus was exposed to the empty gel and enhancing candidate only (tested at highest concentration). This verifies that the alginogel as such does not possess any antimicrobial activity and that glutamine does not possess an antimicrobial effect as such, but is able to augment the antimicrobial effect of the GOX+LPO enzyme solution instead.

2.4.14. Glutamine (P. aeruginosa)

Results indicated that addition of glutamine in a final concentration ranging from 0,0025% to 0,0005% (v/v) to the enzymatic GOX+LPO combination resulted in a difference in absolute microbial counts when compared to that of the reference (no potentiator candidate), illustrating a difference in P. aeruginosa susceptibility (FIG. 31):
Glutamine (0,0025%): resulted in an immediate entire reduction in absolute P. aeruginosa counts when compared to the that of the reference in a 0,0075% (v/v) GOX+LPO enzyme solution concentration. In other words, P. aeruginosa showed to be most susceptible when glutamine (0,0025%) was added to the alginogel.
Glutamine (0,00125-0,0005%): resulted in a faster complete reduction in absolute P. aeruginosa counts when compared to the that of the reference in a 0,0075% (v/v) GOX+LPO enzyme solution concentration. In other words, P. aeruginosa showed to be more susceptible when glutamine (0,00125-0,0005%) was added to the alginogel.

Further, it was indicated that the addition of glutamine in a final concentration of 0.0005% resulted in a faster complete reduction in absolute P. aeruginosa counts compared to when guaiacol was added in the same concentration (i.e., 0.0005%). In other words, P. aeruginosa showed to be more susceptible to the alginogel including the enzymatic GOX+LPO combination and glutamine than that including guaiacol.

Finally, there was no antimicrobial activity when P. aeruginosa was exposed to the empty gel and enhancing candidate only (tested at highest concentration). This verifies that the alginogel as such does not possess any antimicrobial activity and that glutamine does not possess an antimicrobial effect as such, but is able to augment the antimicrobial effect of the GOX+LPO enzyme solution instead.

2.4.15. Glutamine (C. albicans)

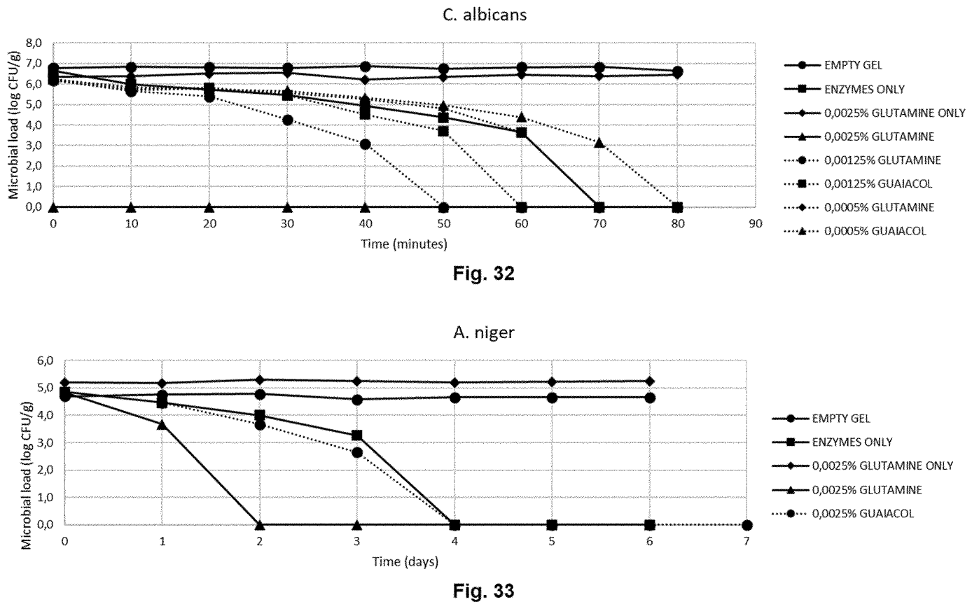

Results indicated that addition of glutamine in a final concentration ranging from 0,0025 to 0,0005% (v/v) to the enzymatic GOX+LPO combination resulted in a difference in absolute microbial counts when compared to that of the reference (no potentiator candidate), illustrating a difference in C. albicans susceptibility (FIG. 32):
Glutamine (0,0025%): resulted in an immediate entire reduction in absolute C. albicans counts when compared to the that of the reference in a 0,0075% (v/v) GOX+LPO enzyme solution concentration. In other words, C. albicans showed to be most susceptible when glutamine (0,0025%) was added to the alginogel.
Glutamine (0,00125-0,0005%): resulted in a faster complete reduction in absolute C. albicans counts when compared to the that of the reference in a 0,0075% (v/v) GOX+LPO enzyme solution concentration. In other words, C. albicans showed to be more susceptible when glutamine (0,00125-0,0005%) was added to the alginogel.

Further, it was indicated that the addition of glutamine in a final concentration of 0.0005% resulted in a faster complete reduction in absolute C. albicans counts compared to when guaiacol was added in the same concentration (i.e., 0.0005%). In other words, C. albicans showed to be more susceptible to the alginogel including the enzymatic GOX+LPO combination and glutamine than that including guaiacol.

Finally, there was no antimicrobial activity when C. albicans was exposed to the empty gel and enhancing candidate only (tested at highest concentration). This verifies that the alginogel as such does not possess any antimicrobial activity and that glutamine does not possess an antimicrobial effect as such, but is able to augment the antimicrobial effect of the GOX+LPO enzyme solution instead.

2.4.16. Glutamine (A. niger)

Results indicated that addition of glutamine in a final concentration of 0,0025% (v/v) to the enzymatic GOX+LPO combination resulted in a difference in absolute microbial counts when compared to that of the reference (no potentiator candidate), illustrating a difference in A. niger susceptibility (FIG. 33):
Glutamine (0,0025%): resulted in a faster complete reduction in absolute A. niger counts when compared to the that of the reference in a 0,0075% (v/v) GOX+LPO enzyme solution concentration. In other words, A. niger showed to be more susceptible when glutamine (0,0025%) was added to the alginogel.

Further, it was indicated that the addition of glutamine in a final concentration of 0.0025% resulted in a faster complete reduction in absolute A. niger counts compared to when guaiacol was added in the same concentration (i.e., 0.0025%). In other words, A. niger showed to be more susceptible to the alginogel including the enzymatic GOX+LPO combination and glutamine than that including guaiacol.

Finally, there was no antimicrobial activity when A. niger was exposed to the empty gel and enhancing candidate only (tested at highest concentration). This verifies that the alginogel as such does not possess any antimicrobial activity and that glutamine does not possess an antimicrobial effect as such, but is able to augment the antimicrobial effect of the GOX+LPO enzyme solution instead.

2.4.17. Asparagine (S. aureus)

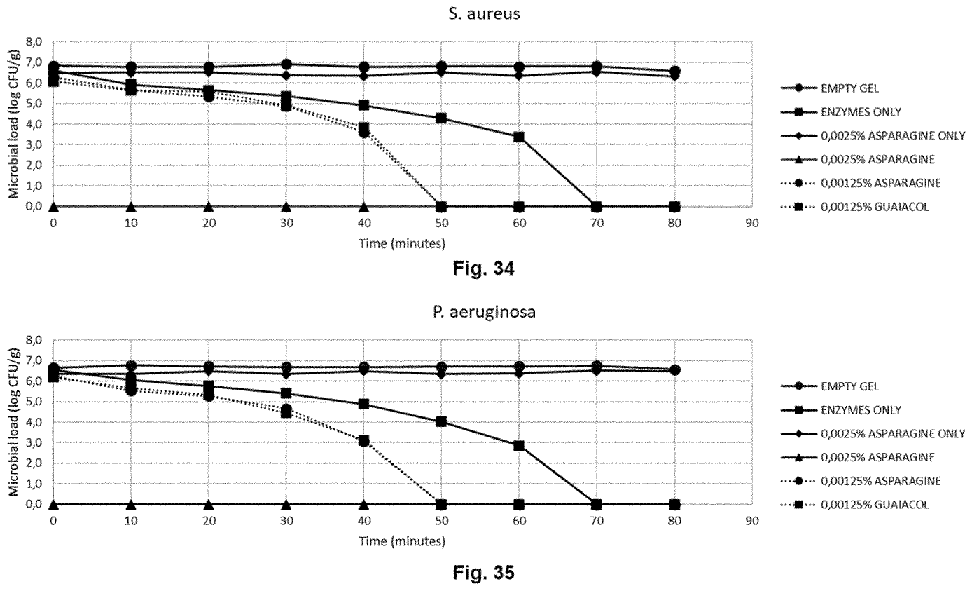

Results indicated that addition of asparagine in a final concentration ranging from 0,0025 to 0,00125% (v/v) to the enzymatic GOX+LPO combination resulted in a difference in antimicrobial efficacy when compared to that of the reference (i.e., enzymes only), illustrating a difference in S. aureus susceptibility (FIG. 34):
Asparagine (0,0025%): resulted in an immediate entire reduction in absolute S. aureus counts when compared to the that of the reference in a 0,0075% (v/v) GOX+LPO enzyme solution concentration. In other words, S. aureus showed to be most susceptible when asparagine (0,0025%) was added to the alginogel.
Asparagine (0,00125%): resulted in a faster complete reduction in absolute S. aureus counts when compared to the that of the reference in a 0,0075% (v/v) GOX+LPO enzyme solution concentration. In other words, S. aureus showed to be more susceptible when asparagine (0,00125%) was added to the alginogel.

Finally, there was no antimicrobial activity when S. aureus was exposed to the empty gel and enhancing candidate only (tested at highest concentration). This verifies that the alginogel as such does not possess any antimicrobial activity and that asparagine does not possess an antimicrobial effect as such, but is able to augment the antimicrobial effect of the GOX+LPO enzyme solution instead.

2.4.18. Asparagine (P. aeruginosa)

Results indicated that addition of asparagine in a final concentration ranging from 0,0025 to 0,00125% (v/v) to the enzymatic GOX+LPO combination resulted in a difference in absolute microbial counts when compared to that of the reference (no potentiator candidate), illustrating a difference in P. aeruginosa susceptibility (FIG. 35):
Asparagine (0,0025%): resulted in an immediate entire reduction in absolute P. aeruginosa counts when compared to the that of the reference in a 0,0075% (v/v) GOX+LPO enzyme solution concentration. In other words, P. aeruginosa showed to be most susceptible when asparagine (0,0025%) was added to the alginogel.
Asparagine (0,00125%): resulted in a faster complete reduction in absolute P. aeruginosa counts when compared to the that of the reference in a 0,0075% (v/v) GOX+LPO enzyme solution concentration. In other words, P. aeruginosa showed to be more susceptible when asparagine (0,00125%) was added to the alginogel.

Finally, there was no antimicrobial activity when P. aeruginosa was exposed to the empty gel and enhancing candidate only (tested at highest concentration). This verifies that the alginogel as such does not possess any antimicrobial activity and that asparagine does not possess an antimicrobial effect as such, but is able to augment the antimicrobial effect of the GOX+LPO enzyme solution instead.

2.4.19. Asparagine (C. albicans)

Results indicated that addition of asparagine in a final concentration ranging from 0,0025 to 0,00125% (v/v) to the enzymatic GOX+LPO combination resulted in a difference in absolute microbial counts when compared to that of the reference (no potentiator candidate), illustrating a difference in C. albicans susceptibility (FIG. 36):
Asparagine (0,0025%): resulted in an immediate entire reduction in absolute C. albicans counts when compared to the that of the reference in a 0,0075% (v/v) GOX+LPO enzyme solution concentration. In other words, C. albicans showed to be most susceptible when asparagine (0,0025%) was added to the alginogel.
Asparagine (0,00125%): resulted in a faster complete reduction in absolute C. albicans counts when compared to the that of the reference in a 0,0075% (v/v) GOX+LPO enzyme solution concentration. In other words, C. albicans showed to be more susceptible when asparagine (0,00125%) was added to the alginogel.

Finally, there was no antimicrobial activity when C. albicans was exposed to the empty gel and enhancing candidate only (tested at highest concentration). This verifies that the alginogel as such does not possess any antimicrobial activity and that asparagine does not possess an antimicrobial effect as such, but is able to augment the antimicrobial effect of the GOX+LPO enzyme solution instead.

2.4.20. Asparagine (A. niger)

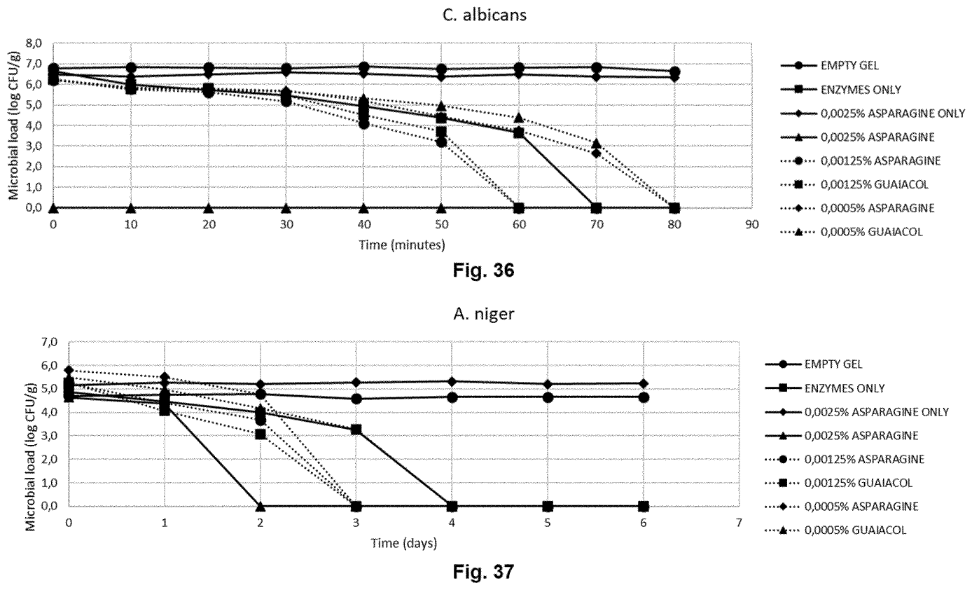

Results indicated that addition of asparagine in a final concentration ranging from 0,0025 to 0,0005% (v/v) to the enzymatic GOX+LPO combination resulted in a difference in absolute microbial counts when compared to that of the reference (no potentiator candidate), illustrating a difference in A. niger susceptibility (FIG. 37):
Asparagine (0,0025-0,0005%): resulted in a faster complete reduction in absolute A. niger counts when compared to the that of the reference in a 0,0075% (v/v) GOX+LPO enzyme solution concentration. In other words, A. niger showed to be more susceptible when asparagine (0,0025-0,0005%) was added to the alginogel.

Further, it was indicated that the addition of asparagine in a final concentration of 0.0005% resulted in a faster complete reduction in absolute A. niger counts compared to when guaiacol was added in the same concentration (i.e., 0.0005%). In other words, A. niger showed to be more susceptible to the alginogel including the enzymatic GOX+LPO combination and asparagine than that including guaiacol.

Finally, there was no antimicrobial activity when A. niger was exposed to the empty gel and enhancing candidate only (tested at highest concentration). This verifies that the alginogel as such does not possess any antimicrobial activity and that asparagine does not possess an antimicrobial effect as such, but is able to augment the antimicrobial effect of the GOX+LPO enzyme solution instead.

2.4.21. Isoleucine (S. aureus)

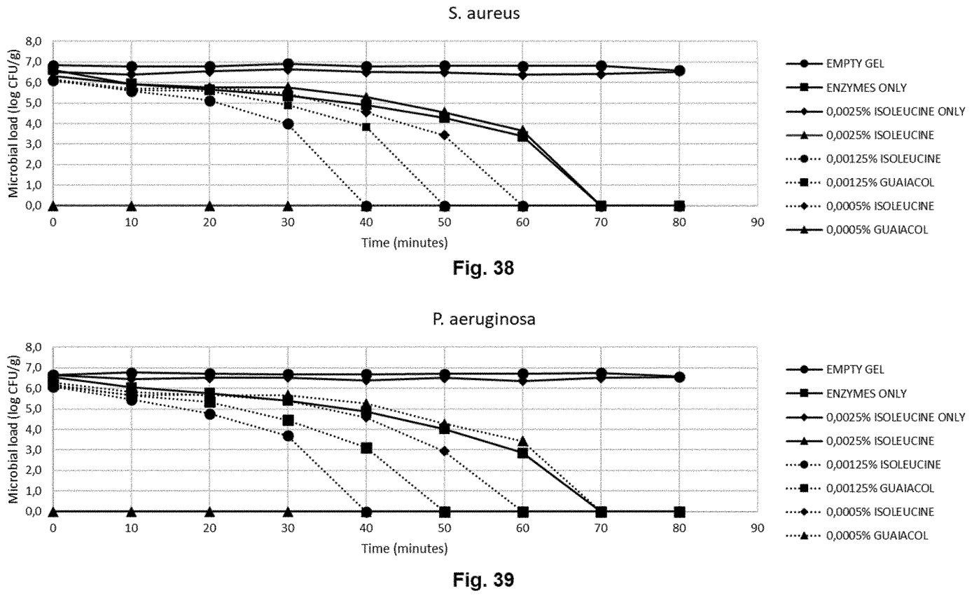

Results indicated that addition of isoleucine in a final concentration ranging from 0,0025 to 0,0005% (v/v) to the enzymatic GOX+LPO combination resulted in a difference in antimicrobial efficacy when compared to that of the reference (i.e., enzymes only), illustrating a difference in S. aureus susceptibility (FIG. 38):
Isoleucine (0,0025%): resulted in an immediate entire reduction in absolute S. aureus counts when compared to the that of the reference in a 0,0075% (v/v) GOX+LPO enzyme solution concentration. In other words, S. aureus showed to be most susceptible when isoleucine (0,0025%) was added to the alginogel.
Isoleucine (0,00125-0,0005%): resulted in a faster complete reduction in absolute S. aureus counts when compared to the that of the reference in a 0,0075% (v/v) GOX+LPO enzyme solution concentration. In other words, S. aureus showed to be more susceptible when isoleucine (0,00125-0,0005%) was added to the alginogel.

Further, it was indicated that the addition of isoleucine in a final concentration of 0.0005% resulted in a faster complete reduction in absolute S. aureus counts compared to when guaiacol was added in the same concentration (i.e., 0.0005%). In other words, S. aureus showed to be more susceptible to the alginogel including the enzymatic GOX+LPO combination and isoleucine than that including guaiacol.

Finally, there was no antimicrobial activity when S. aureus was exposed to the empty gel and enhancing candidate only (tested at highest concentration). This verifies that the alginogel as such does not possess any antimicrobial activity and that isoleucine does not possess an antimicrobial effect as such, but is able to augment the antimicrobial effect of the GOX+LPO enzyme solution instead.

2.4.22. Isoleucine (P. aeruginosa)

Results indicated that addition of isoleucine in a final concentration ranging from 0,0025 to 0,0005% (v/v) to the enzymatic GOX+LPO combination resulted in a difference in absolute microbial counts when compared to that of the reference (no potentiator candidate), illustrating a difference in P. aeruginosa susceptibility (FIG. 39):
Isoleucine (0,0025%): resulted in an immediate entire reduction in absolute P. aeruginosa counts when compared to the that of the reference in a 0,0075% (v/v) GOX+LPO enzyme solution concentration. In other words, P. aeruginosa showed to be most susceptible when isoleucine (0,0025%) was added to the alginogel.
Isoleucine (0,00125-0,0005%): resulted in a faster complete reduction in absolute P. aeruginosa counts when compared to the that of the reference in a 0,0075% (v/v) GOX+LPO enzyme solution concentration. In other words, P. aeruginosa showed to be more susceptible when isoleucine (0,00125-0,0005%) was added to the alginogel.

Further, it was indicated that the addition of isoleucine in a final concentration of 0.0005% resulted in a faster complete reduction in absolute P. aeruginosa counts compared to when guaiacol was added in the same concentration (i.e., 0.0005%). In other words, P. aeruginosa showed to be more susceptible to the alginogel including the enzymatic GOX+LPO combination and isoleucine than that including guaiacol.

Finally, there was no antimicrobial activity when P. aeruginosa was exposed to the empty gel and enhancing candidate only (tested at highest concentration). This verifies that the alginogel as such does not possess any antimicrobial activity and that isoleucine does not possess an antimicrobial effect as such, but is able to augment the antimicrobial effect of the GOX+LPO enzyme solution instead.

2.4.23. Isoleucine (C. albicans)

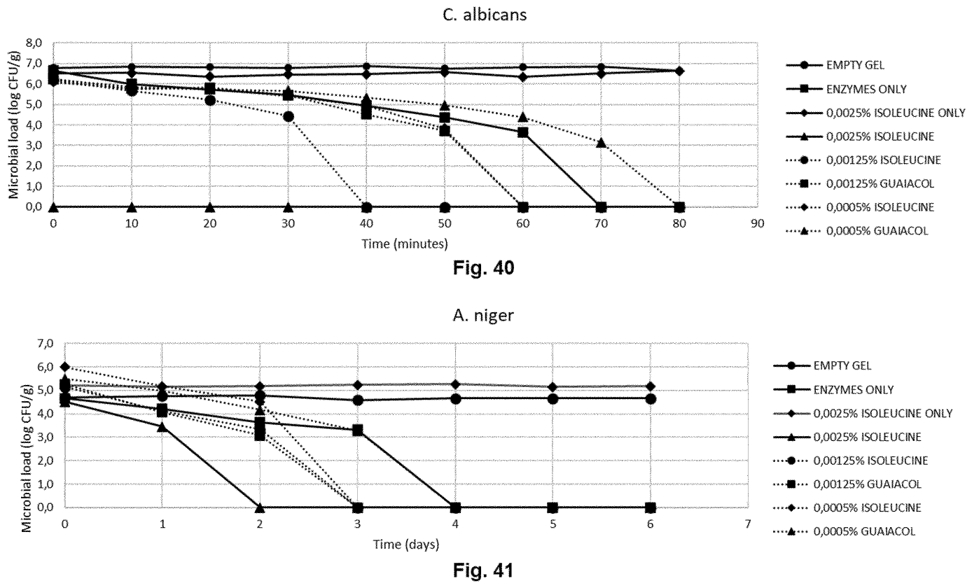

Results indicated that addition of isoleucine in a final concentration ranging from 0,0025 to 0,0005% (v/v) to the enzymatic GOX+LPO combination resulted in a difference in absolute microbial counts when compared to that of the reference (no potentiator candidate), illustrating a difference in C. albicans susceptibility (FIG. 40):
Isoleucine (0,0025%): resulted in an immediate entire reduction in absolute C. albicans counts when compared to the that of the reference in a 0,0075% (v/v) GOX+LPO enzyme solution concentration. In other words, C. albicans showed to be most susceptible when isoleucine (0,0025%) was added to the alginogel.
Isoleucine (0,00125-0,0005%): resulted in a faster complete reduction in absolute C. albicans counts when compared to the that of the reference in a 0,0075% (v/v) GOX+LPO enzyme solution concentration. In other words, C. albicans showed to be more susceptible when isoleucine (0,00125-0,0005%) was added to the alginogel.

Further, it was indicated that the addition of isoleucine in a final concentration of 0.0005% resulted in a faster complete reduction in absolute C. albicans counts compared to when guaiacol was added in the same concentration (i.e., 0.0005%). In other words, P. aeruginosa showed to be more susceptible to the alginogel including the enzymatic GOX+LPO combination and isoleucine than that including guaiacol.

Finally, there was no antimicrobial activity when C. albicans was exposed to the empty gel and enhancing candidate only (tested at highest concentration). This verifies that the alginogel as such does not possess any antimicrobial activity and that isoleucine does not possess an antimicrobial effect as such, but is able to augment the antimicrobial effect of the GOX+LPO enzyme solution instead.

2.4.24. Isoleucine (A. niger)

Results indicated that addition of isoleucine in a final concentration ranging from 0,0025 to 0,0005% (v/v) to the enzymatic GOX+LPO combination resulted in a difference in absolute microbial counts when compared to that of the reference (no potentiator candidate), illustrating a difference in A. niger susceptibility (FIG. 41):
Isoleucine (0,0025-0,0005%): resulted in a faster complete reduction in absolute A. niger counts when compared to the that of the reference in a 0,0075% (v/v) GOX+LPO enzyme solution concentration. In other words, A. niger showed to be more susceptible when isoleucine (0,0025-0,0005%) was added to the alginogel.

Further, it was indicated that the addition of isoleucine in a final concentration of 0.0005% resulted in a faster complete reduction in absolute A. niger counts compared to when guaiacol was added in the same concentration (i.e., 0.0005%). In other words, A. niger showed to be more susceptible to the alginogel including the enzymatic GOX+LPO combination and isoleucine than that including guaiacol.

Finally, there was no antimicrobial activity when A. niger was exposed to the empty gel and enhancing candidate only (tested at highest concentration). This verifies that the alginogel as such does not possess any antimicrobial activity and that isoleucine does not possess an antimicrobial effect as such, but is able to augment the antimicrobial effect of the GOX+LPO enzyme solution instead.

### Example 3. Comparison of Ethanolamine and Proline

The purpose of the hereby described experiment was to compare the efficacy of antimicrobial activity enhancement of the GOX+LPO (1:1) enzyme combination by ethanolamine and proline in a microbial challenge test using S. aureus. The test was set up as described in the section on ‘challenge testing’ in Example 2.4. The test was repeated at least 3 times.

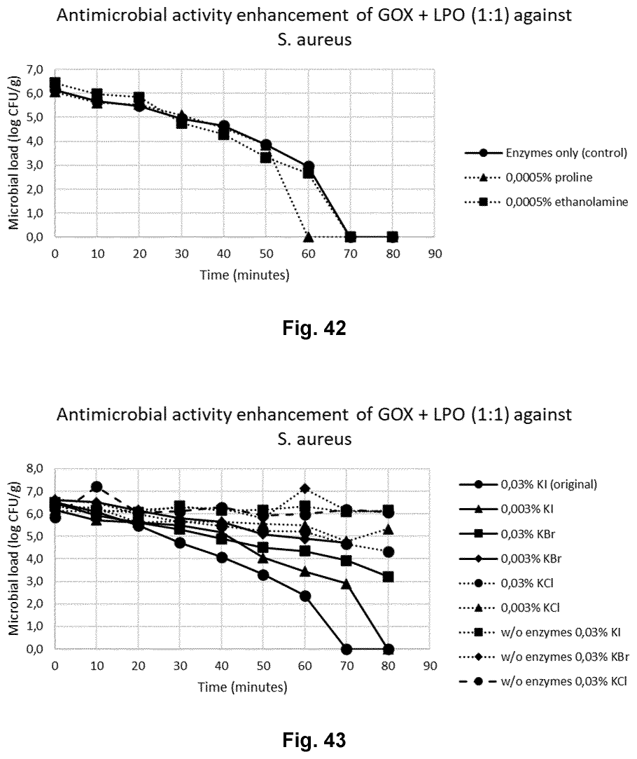

The results indicated that addition of proline at a final concentration of 0,0005% (v/v) to the alginogel with GOX+LPO (1:1) enzyme combination resulted in a difference in antimicrobial efficacy against S. aureus when compared to that of the reference (i.e., alginogel with GOX+LPO enzyme combination only) (FIG. 42). Proline (0,0005%) resulted in a faster complete reduction of absolute S. aureus counts (average of 60 min) when compared to that of the reference to which no enhancement molecule was added (average of 70 min). In other words, S. aureus showed to be more susceptible when proline (0,0005%) was added to the alginogel.

However, addition of ethanolamine at a final concentration of 0,0005% (v/v) to the alginogel with GOX+LPO (1:1) enzyme combination did not result in a difference in antimicrobial efficacy when compared to that of the reference (i.e., alginogel with GOX+LPO enzyme combination only), illustrating that there is no difference in S. aureus susceptibility (FIG. 42). Both required a similar exposure time to ensure complete reduction of absolute S. aureus counts (average of 70 min). In other words, whether or not ethanolamine was added to the alginogel, S. aureus remained equally susceptible.

It can be concluded that based on the current test set-up, proline was found to be more effective as antimicrobial activity enhancement molecule of the GOX+LPO enzyme combination than ethanolamine.

### Example 4. Comparison of Different Halides

The purpose of the hereby described experiment was to compare the efficacy of antimicrobial activity of the GOX+LPO (1:1) enzyme combination in the presence of potassium chloride/potassium bromide and potassium iodide. A microbial challenge test using S. aureus was performed. The test was set up as described in the section on ‘challenge testing’ in in Example 2.4.

The results indicated that addition of KI at a final concentration of 0,03-0,003% (v/v) to the alginogel with GOX+LPO (1:1) enzyme combination resulted in a difference in antimicrobial efficacy against S. aureus when compared to the addition of KBr or KCl in 0,03-0,003% (v/v) (FIG. 43). KI (0,03-0,003%) resulted in a faster complete reduction of absolute S. aureus counts (respectively 70-80 min) when compared to addition of KBr and KCl (0,03-0,003%), where no complete killing occurred within the 80-minute timeframe. In other words, S. aureus showed to be more susceptible when KI (0,03-0,003%) was added to the alginogel.

Addition of KI, KBr and KCl alone (without enzymes) at a final concentration of 0.03% (v/v) to the alginogel with GOX+LPO (1:1) enzyme combination did not result in any difference in antimicrobial efficacy over the 80 minute timeframe when comparing the 0 and 80 minute timepoint, illustrating that there is no difference in S. aureus susceptibility (FIG. 43). This further validates that the halides used are not antimicrobial as such and solely play a substrate role for the peroxidase complex.

It can be concluded that based on the current test set-up, the antimicrobial activity of the GOX+LPO (1:1) enzyme combination was found to be more effective in the presence of KI than in the presence of KBr and KCl.

### Example 5. Characterisation of the Anti-Inflammatory Effect of the Composition

To compare the inflammatory modulation activity of the GOX+LPO (1:1) enzyme combination with and without increasing concentrations of the enhancing molecule isoleucine, 3 types of pro-inflammatory mediators were studied, i.e., IL-6 (interleukin), TNFa (non-interleukin cytokine) and MMP9 (enzyme). First, the effect on the secretion of these mediators by macrophages was evaluated. Secondly, the direct neutralization of already secreted mediators was evaluated. Both were measured by the state-of-the-art ELISA technique.

First, the secretion of the pro-inflammatory mediator representatives IL-6, TNFa and MMP9 in cell culture supernatants by THP-1 (a monocytic cell line) after its differentiation into macrophages were measured. In brief, THP-1 cells were seeded at a density of 3×105 cells/cm² in a 6-well plate in the presence of 160 nM of phorbol-12-myristate-13-acetate (PMA) to induce macrophage differentiation. Differentiation was further conducted for 48 h. Macrophages were then treated with the GOX+LPO (1:1) enzyme combination, their substrate (i.e., glucose and potassium iodide) and different concentrations (i.e., 0.0025%, 0.05% and 0.5%) of the enhancing molecule isoleucine in serum-free medium for 24 h at 37° C. Supernatants were collected and centrifuged at 150 g for 5 min to remove cell debris and were kept at −20° C. before being processed for ELISA.

Secondly, the direct neutralization of secreted pro-inflammatory mediator representatives IL-6, TNFa and MMP9 was measured in parallel. In brief, cell culture supernatants were collected after 48 h of macrophage differentiation and were centrifuged at 150 g for 5 min to remove cell debris. Then, the cell culture supernatants were treated with GOX+LPO (1:1) enzyme combination, their substrate (i.e., glucose and potassium iodide) and 0.05% of the enhancing molecule isoleucine for 3 h at room temperature. Treated supernatants were kept frozen until being processed for ELISA.

IL-6, TNFa and MMP9 ELISA kits were purchased from BioTechne and the experiment was performed according to manufacturer's instructions. Briefly, 96-well plates were coated with capture antibodies overnight at room temperature. After incubating the plate for 1 h with a blocking solution, supernatants were added in the well for 2 h at room temperature. After several washing steps, the corresponding detection antibody, which is coupled to biotin, was incubated for 2 h. Streptavidin coupled to horseradish peroxidase (HRP) was added for 20 min and HRP substrate solution was added for another 20 min. The reaction was stopped by the addition of sulphuric acid and absorbance at 450 nm was read with a plate reader. Absorbance is proportional to the concentration of analyte present in the medium, which was calculated based on a standard curve realized with recombinant IL-6, TNFa and MMP9.

The tests were repeated at least once to obtain insights on preliminary results.

### IL-6 Mediator

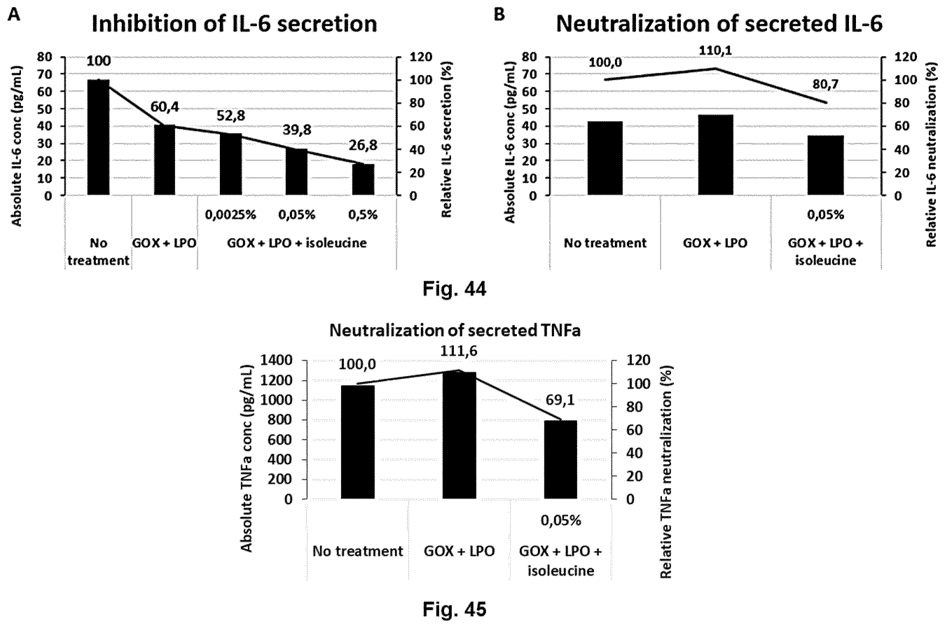

The results indicated that the addition of the enhancing molecule isoleucine in a final concentration ranging from 0.5 to 0.0025% (v/v) to the GOX+LPO (1:1) enzyme combination resulted in an additional difference in IL-6 secretion by macrophages (FIG. 44A). When compared to macrophages treated with the GOX+LPO (1:1) enzyme combination alone, an additional decrease of 7.6, 20.6 and 33.6% was observed when 0.0025, 0.05 and 0.5% isoleucine, respectively, was added (FIG. 44A).

The results further indicated that the addition of the enhancing molecule isoleucine in a final concentration of 0.05% (v/v) to the GOX+LPO (1:1) enzyme combination resulted in an additional difference in direct neutralization of already secreted IL-6 (FIG. 44B). When compared to supernatants treated with the GOX+LPO (1:1) enzyme combination alone, an additional decrease of 29.4% was observed when 0.05% isoleucine was added (FIG. 44B).

As such, these results illustrate that there is a difference in IL-6 modulation activity of the GOX+LPO (1:1) enzyme combination in the presence and absence of increasing concentrations of the enhancing molecule isoleucine.

### TNFa Mediator

For TNFa, the results indicated that the addition of the enhancing molecule isoleucine in a final concentration of 0.05% (v/v) to the GOX+LPO (1:1) enzyme combination resulted in an additional difference on the level of direct neutralization of already secreted TNFa (FIG. 45). When compared to supernatants treated with the GOX+LPO (1:1) enzyme combination alone, an additional decrease of 42.5% was observed when 0.05% isoleucine was added (FIG. 45).

As such, these results illustrate that there is a difference in TNFa modulation activity of the GOX+LPO (1:1) enzyme combination in the presence and absence of increasing concentrations of the enhancing molecule isoleucine.

### MMP9 Mediator

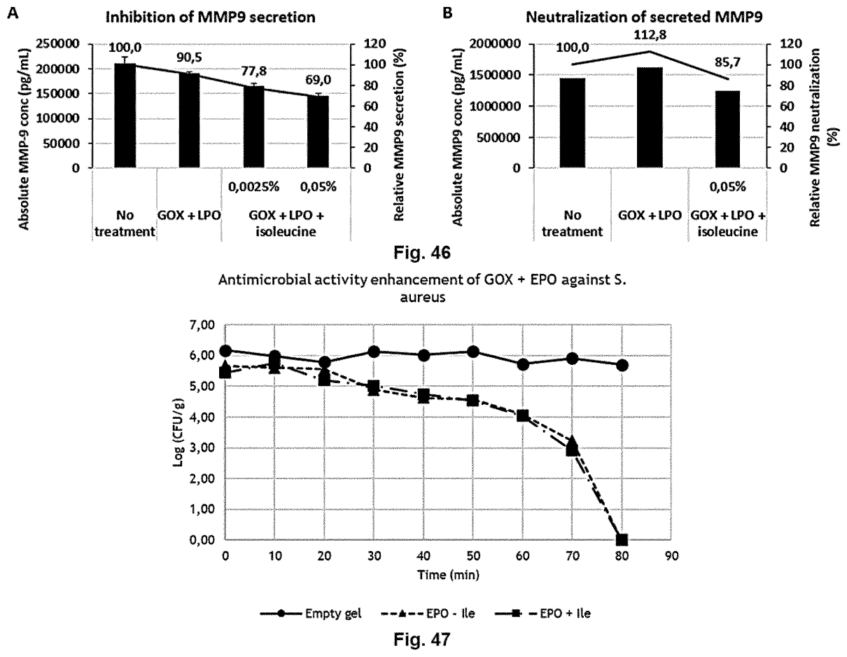

The results indicated that the addition of the enhancing molecule isoleucine in a final concentration ranging from 0.05 to 0.0025% (v/v) to the GOX+LPO (1:1) enzyme combination resulted in an additional difference in MMP9 secretion by macrophages (FIG. 46A). When compared to macrophages treated with the GOX+LPO (1:1) enzyme combination alone, an additional decrease of 12.7 and 21.5% was observed when 0.0025 and 0.05% isoleucine, respectively, was added (FIG. 46A).

The results further indicated that the addition of the enhancing molecule isoleucine in a final concentration of 0.05% (v/v) to the GOX+LPO (1:1) enzyme combination resulted in an additional difference in direct neutralization of already secreted MMP9 (FIG. 46B). When compared to supernatants treated with the GOX+LPO (1:1) enzyme combination alone, an additional decrease of 27.1% was observed when 0.05% isoleucine was added (FIG. 46B).

As such, these results illustrate that there is a difference in MMP9 modulation activity of the GOX+LPO (1:1) enzyme combination in the presence and absence of increasing concentrations of the enhancing molecule isoleucine.

It can be concluded that based on the current test set-up, the GOX+LPO (1:1) enzyme combination is found to be sensitive to enhancement of its modulation activity against 3 types of pro-inflammatory mediator representatives, i.e., IL-6 (interleukin), TNFa (non-interleukin cytokine) and MMP9 (enzyme), by isoleucine. For IL-6 and MMP9, both an inhibition of secretion by macrophages as well as direct neutralization of already secreted mediators were observed. For TNFa, a direct neutralization effect of the already secreted mediator was observed.

### Example 6. The Effect of the Enhancing Agents Upon Using Eosinophil Peroxidase in the Composition

In a further experiment, the efficacy of antimicrobial activity enhancement by isoleucine of the GOX combination with eosinophil peroxidase (EPO) was evaluated. In view hereof, a test was set up as described in the section on ‘challenge testing’ described above with the difference that EPO was used instead of LPO.

In brief, microbial challenge testing was performed according to the method described in the European Pharmacopoeia current version chapter 5.1.3 ‘Efficacy of antimicrobial preservation’, but with adapted time points (0, 10, 20, 30, 40, 50, 60, 70 and 80 min instead of 0, 2, 7, 14 and 28 days). A lab-scale alginogel (based on Flaminal® formulation) with 0.00001875% (w/w) GOX and 0.000005% (w/w) EPO (Creative enzymes NATE-0228), was prepared. Isoleucine was added to the alginogel at a final concentration of 0.0025% (w/w). As a reference, an alginogel including 0.00001875% GOX+0.000005% EPO without the addition of isoleucine was included. As a negative control, an alginogel without both GOX+EPO enzyme combination and without potentiator candidate (empty gel) was included. After preparation, a homogenous sample of 10 g was transferred into a 50 ml conical tube for each alginogel to be evaluated. For the test microorganism, i.e. S. aureus (ATCC®6538™), a microbial suspension of about 108 CFU/ml was prepared in sterile diluent (0.9% (w/v) NaCl). Then, the 50 ml conical tubes containing the alginogel samples to be evaluated were inoculated with 100 μl of the designated inoculum and incubated at 25° C. for 80 minutes. At the start (time point 0) and after 0, 10, 20, 30, 40, 50, 60, 70 and 80 min, the number of viable microorganisms per gram product (expressed as CFU/g) was determined by transferring a sample of 0.5 g of the product into a 15 ml conical tube containing 4.5 ml of sterile diluent and applying general dilution and plate count techniques. For each combination evaluated, the absolute counts (expressed as log(CFU/g)) at each timepoint were visualized in a graph. The test was repeated once to obtain insights on preliminary results.

The results indicated that addition of isoleucine at a final concentration of 0.0025% (w/w) to the alginogel with GOX+EPO enzyme combinations did not result in a difference in antimicrobial efficacy against S. aureus when compared to that of the reference (i.e. alginogel with GOX+EPO enzyme combination only) (FIG. 47). Isoleucine (0.0025%) did not result in a faster complete reduction of absolute S. aureus counts (80 min) when compared to the reference alginogel to which no enhancement molecule was added (80 min). In other words, isoleucine does not enhance the antimicrobial activity of GOX+EPO.

1. A pharmaceutical and/or cosmetic composition comprising:
a peroxide generating system;
a lactoperoxidase;
a halide or pseudohalide;
and an enhancing agent;
wherein the enhancing agent is a hydrophilic amino acid, amino acid mimic, or amino acid derivative, characterized by a free amino functional group (—NH₃+), free carboxylic acid functional group (—CO₂H), and a side chain comprising an
(i) —OH group;
(ii) —(CH₂)ₙOH group wherein n is 1, 2, or 3;
(iii) —(CH₂)ₙCONH₂ group wherein n is 1, 2, or 3;
(iv) -a cyclic C₄H₉N group; or
(v) a branched alkylgroup.

2. The composition according to claim 1, wherein the enhancing agent is a hydrophilic amino acid, amino acid mimic, or amino acid derivative having an octanol/water partition coefficient of from about −1 to about −5.

3. The composition according to claim 1, wherein the enhancing agent is a hydrophilic naturally or non-naturally occurring amino acid.

4. The composition according to claim 1, wherein the enhancing agent is a naturally occurring amino acid having an octanol/water partition coefficient of from −1.72 to −3.82.

5. The composition according to claim 1, wherein the halide or pseudohalide is a water-soluble iodide salt.

6. The composition according to claim 1, wherein the enhancing agent is present in said composition at a concentration of from 0.0001 mg/ml to 500 mg/ml.

7. The composition according to claim 1, wherein the peroxide generating system comprises an oxidase.

8. The composition according to claim 1, wherein the lactoperoxidase comprises an amino acid sequence having at least 90%.

9. The composition according to claim 1, wherein said composition comprises:
from about 150 to about 4000 U/kg of the peroxide generating system;
from about 10 to about 100000 U/kg of the lactoperoxidase;
from about 0.01 to about 500 mg/kg of the halide or pseudohalide;
and from 0.0005 mg/ml to 200 mg/ml of the enhancing agent.

10. A pharmaceutical formulation, comprising: the composition according to claim 1 and one or more pharmaceutically acceptable carriers.

11.-19. (canceled)

20. A method of treating or preventing a skin disorder in a subject, the method comprising:
administering to a subject in need thereof the composition according to claim 1.

21. A method of healing one or more wounds of a subject, the method comprising:
administering to a subject in need thereof the composition according to claim 1.

22. A method of treating or preventing a microbial infection in a subject, the method comprising:
administering to a subject in need thereof the composition according to claim 1.

23.-25. (canceled)

26. The composition according to claim 1, wherein the enhancing agent is a naturally occurring amino acid selected from serine, threonine, asparagine, glutamine, isoleucine, proline, or any combination thereof.

27. The composition according to claim 7, wherein the oxidase is glucose oxidase.

28. The method of claim 21, wherein the subject has a chronic wound.

29. The method of claim 28, wherein the chronic wound comprises a diabetic foot ulcer, a venous leg ulcer, or pressure ulcer.

30. The method of claim 21, wherein the subject has a burn wound.

31. The method of claim 22, wherein the microbial infection comprises a viral infection, a bacterial infection, a fungal infection, or any combination thereof.

32. The method of claim 22, wherein, following the administering step, inflammation in the subject is reduced.


SEQUENCE LISTING (5 sequences)

SEQ ID NO: 1 - protein, 612 aa, Bos taurus
DTTLTNVTDPSLDLTALSWEVGCGAPVPLVKCDENSPYRTITGDCNNRRSPALGAANRALARWLPAEYEDGLALPFGWTQRKTRNGFRVPLAREVSNKIVGYLDEEGVLDQNRSLLFMQWGQIVDHDLDFAPETELGSNEHSKTQCEEYCIQGDNCFPIMFPKNDPKLKTQGKCMPFFRAGFVCPTPPYQSLAREQINAVTSFLDASLVYGSEPSLASRLRNLSSPLGLMAVNQEAWDHGLAYLPFNNKKPSPCEFINTTARVPCFLAGDFRASEQILLATAHTLLLREHNRLARELKKLNPHWNGEKLYQEARKILGAFIQIITFRDYLPIVLGSEMQKWIPPYQGYNNSVDPRISNVFTFAFRFGHMEVPSTVSRLDENYQPWGPEAELPLHTLFFNTWRIIKDGGIDPLVRGLLAKKSKLMNQDKMVTSELRNKLFQPTHKIHGFDLAAINLQRCRDHGMPGYNSWRGFCGLSQPKTLKGLQTVLKNKILAKKLMDLYKTPDNIDIWIGGNAEPMVERGRVGPLLACLLGRQFQQIRDGDRFWWENPGVFTEKQRDSLQKVSFSRLICDNTHITKVPLHAFQANNYPHDFVDCSTVDKLDLSPWASREN

SEQ ID NO: 2 - protein, 690 aa, Bos taurus
DTIAQAASTTTISDAVSKVKIQVNKAFLDSRTRLKTTLSSEAPTTQQLSEYFKHAKGRTRTAIRNGQVWEESLKRLRRDTTLTNVTDPSLDLTALSWEVGCGAPVPLVKCDENSPYRTITGDCNNRRSPALGAANRALARWLPAEYEDGLALPFGWTQRKTRNGFRVPLAREVSNKIVGYLDEEGVLDQNRSLLFMQWGQIVDHDLDFAPETELGSNEHSKTQCEEYCIQGDNCFPIMFPKNDPKLKTQGKCMPFFRAGFVCPTPPYQSLAREQINAVTSFLDASLVYGSEPSLASRLRNLSSPLGLMAVNQEAWDHGLAYLPFNNKKPSPCEFINTTARVPCFLAGDFRASEQILLATAHTLLLREHNRLARELKKLNPHWNGEKLYQEARKILGAFIQIITFRDYLPIVLGSEMQKWIPPYQGYNNSVDPRISNVFTFAFRFGHMEVPSTVSRLDENYQPWGPEAELPLHTLFFNTWRIIKDGGIDPLVRGLLAKKSKLMNQDKMVTSELRNKLFQPTHKIHGFDLAAINLQRCRDHGMPGYNSWRGFCGLSQPKTLKGLQTVLKNKILAKKLMDLYKTPDNIDIWIGGNAEPMVERGRVGPLLACLLGRQFQQIRDGDRFWWENPGVFTEKQRDSLQKVSFSRLICDNTHITKVPLHAFQANNYPHDFVDCSTVDKLDLSPWASREN

SEQ ID NO: 3 - protein, 712 aa, Bos taurus
MWVCLQLPVFLASVTLFEVAASDTIAQAASTTTISDAVSKVKIQVNKAFLDSRTRLKTTLSSEAPTTQQLSEYFKHAKGRTRTAIRNGQVWEESLKRLRRDTTLTNVTDPSLDLTALSWEVGCGAPVPLVKCDENSPYRTITGDCNNRRSPALGAANRALARWLPAEYEDGLALPFGWTQRKTRNGFRVPLAREVSNKIVGYLDEEGVLDQNRSLLFMQWGQIVDHDLDFAPETELGSNEHSKTQCEEYCIQGDNCFPIMFPKNDPKLKTQGKCMPFFRAGFVCPTPPYQSLAREQINAVTSFLDASLVYGSEPSLASRLRNLSSPLGLMAVNQEAWDHGLAYLPFNNKKPSPCEFINTTARVPCFLAGDFRASEQILLATAHTLLLREHNRLARELKKLNPHWNGEKLYQEARKILGAFIQIITFRDYLPIVLGSEMQKWIPPYQGYNNSVDPRISNVFTFAFRFGHMEVPSTVSRLDENYQPWGPEAELPLHTLFFNTWRIIKDGGIDPLVRGLLAKKSKLMNQDKMVTSELRNKLFQPTHKIHGFDLAAINLQRCRDHGMPGYNSWRGFCGLSQPKTLKGLQTVLKNKILAKKLMDLYKTPDNIDIWIGGNAEPMVERGRVGPLLACLLGRQFQQIRDGDRFWWENPGVFTEKQRDSLQKVSFSRLICDNTHITKVPLHAFQANNYPHDFVDCSTVDKLDLSPWASREN

SEQ ID NO: 4 - protein, 22 aa, synthetic construct
MWVCLQLPVFLASVTLFEVAAS

SEQ ID NO: 5 - protein, 78 aa, synthetic construct
DTIAQAASTTTISDAVSKVKIQVNKAFLDSRTRLKTTLSSEAPTTQQLSEYFKHAKGRTRTAIRNGQVWEESLKRLRR